SEQUENCE LISTING (376 sequences)

SEQ ID NO: 1 - DNA, 2484 bp, Homo sapiens
  source 1..2484: mol_type = genomic DNA
gatgaatctgcactgacccttggtacaatagatgtttcttatctgccacattcatcagaatacagtgttggtcgatgtaagcacacaagtgaggaatggggtgagtgtggctttagacccaccatcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtag

SEQ ID NO: 2 - RNA, 19 nt, synthetic construct
  source 1..19: mol_type = other RNA
gacccttggtacaatagat

SEQ ID NO: 3 - RNA, 19 nt, synthetic construct
  source 1..19: mol_type = other RNA
caggcaataatctcaacat

SEQ ID NO: 4 - RNA, 19 nt, synthetic construct
  source 1..19: mol_type = other RNA
ggcatatagttgaattact

SEQ ID NO: 5 - RNA, 19 nt, synthetic construct
  source 1..19: mol_type = other RNA
cctggagcaagcgttgtta

SEQ ID NO: 6 - RNA, 19 nt, synthetic construct
  source 1..19: mol_type = other RNA
ggtagagacacgtccatta

SEQ ID NO: 7 - RNA, 19 nt, synthetic construct
  source 1..19: mol_type = other RNA
gctcgatgaaacaccagat

SEQ ID NO: 8 - RNA, 19 nt, synthetic construct
  source 1..19: mol_type = other RNA
cctcaatgcaaccgacaaa

SEQ ID NO: 9 - RNA, 19 nt, synthetic construct
  source 1..19: mol_type = other RNA
ccggcaatgatcagactga

SEQ ID NO: 10 - RNA, 19 nt, synthetic construct
  source 1..19: mol_type = other RNA
ggtcgatgtaagcacacaa

SEQ ID NO: 11 - RNA, 19 nt, synthetic construct
  source 1..19: mol_type = other RNA
gatcccatattgactatct

SEQ ID NO: 12 - RNA, 19 nt, synthetic construct
  source 1..19: mol_type = other RNA
gcatatagttgaattactt

SEQ ID NO: 13 - RNA, 19 nt, synthetic construct
  source 1..19: mol_type = other RNA
ctttcttacgttcttttta

SEQ ID NO: 14 - RNA, 19 nt, synthetic construct
  source 1..19: mol_type = other RNA
gaatgttatttatatcaat

SEQ ID NO: 15 - RNA, 19 nt, synthetic construct
  source 1..19: mol_type = other RNA
gacaaaaacttctagaata

SEQ ID NO: 16 - RNA, 19 nt, synthetic construct
  source 1..19: mol_type = other RNA
gaaacagtaggaaagttta

SEQ ID NO: 17 - RNA, 19 nt, synthetic construct
  source 1..19: mol_type = other RNA
gtacaatagatgtttctta

SEQ ID NO: 18 - RNA, 19 nt, synthetic construct
  source 1..19: mol_type = other RNA
caatcaaaagccgttaaca

SEQ ID NO: 19 - RNA, 19 nt, synthetic construct
  source 1..19: mol_type = other RNA
ggaatgttatttatatcaa

SEQ ID NO: 20 - RNA, 19 nt, synthetic construct
  source 1..19: mol_type = other RNA
gaaagatgttctctataga

SEQ ID NO: 21 - RNA, 19 nt, synthetic construct
  source 1..19: mol_type = other RNA
ctgggaaattgtgtcacaa

SEQ ID NO: 22 - RNA, 19 nt, synthetic construct
  source 1..19: mol_type = other RNA
gcttgcaagacgcctttct

SEQ ID NO: 23 - RNA, 19 nt, synthetic construct
  source 1..19: mol_type = other RNA
gaggccatcatagcttgca

SEQ ID NO: 24 - RNA, 19 nt, synthetic construct
  source 1..19: mol_type = other RNA
ctggaaggcacacgttcta

SEQ ID NO: 25 - RNA, 19 nt, synthetic construct
  source 1..19: mol_type = other RNA
gtgccattatgtccacaca

SEQ ID NO: 26 - RNA, 19 nt, synthetic construct
  source 1..19: mol_type = other RNA
gccacatattgtcttgtga

SEQ ID NO: 27 - RNA, 19 nt, synthetic construct
  source 1..19: mol_type = other RNA
gtcaacttgagatggttaa

SEQ ID NO: 28 - RNA, 19 nt, synthetic construct
  source 1..19: mol_type = other RNA
cagcagtttatcaccttct

SEQ ID NO: 29 - RNA, 19 nt, synthetic construct
  source 1..19: mol_type = other RNA
gatggttaaagctgcaact

SEQ ID NO: 30 - RNA, 19 nt, synthetic construct
  source 1..19: mol_type = other RNA
gtggtagatactctgtcta

SEQ ID NO: 31 - RNA, 19 nt, synthetic construct
  source 1..19: mol_type = other RNA
cattgcttcaggcaataat

SEQ ID NO: 32 - RNA, 19 nt, synthetic construct
  source 1..19: mol_type = other RNA
cctgtggagtgtagcaaga

SEQ ID NO: 33 - RNA, 19 nt, synthetic construct
  source 1..19: mol_type = other RNA
ctgccatgaaacagtagga

SEQ ID NO: 34 - RNA, 19 nt, synthetic construct
  source 1..19: mol_type = other RNA
ggctcgatgaaacaccaga

SEQ ID NO: 35 - RNA, 19 nt, synthetic construct
  source 1..19: mol_type = other RNA
gaaggaggttgattgcaaa

SEQ ID NO: 36 - RNA, 19 nt, synthetic construct
  source 1..19: mol_type = other RNA
gccattatgtccacacaca

SEQ ID NO: 37 - RNA, 19 nt, synthetic construct
  source 1..19: mol_type = other RNA
caccggcaatgatcagact

SEQ ID NO: 38 - RNA, 19 nt, synthetic construct
  source 1..19: mol_type = other RNA
gtaccttgatccataagct

SEQ ID NO: 39 - RNA, 19 nt, synthetic construct
  source 1..19: mol_type = other RNA
gtagagtacaagaggcaat

SEQ ID NO: 40 - RNA, 19 nt, synthetic construct
  source 1..19: mol_type = other RNA
ccaggaagatatcagtcct

SEQ ID NO: 41 - RNA, 19 nt, synthetic construct
  source 1..19: mol_type = other RNA
gacacgtccattaatgagt

SEQ ID NO: 42 - RNA, 19 nt, synthetic construct
  source 1..19: mol_type = other RNA
gtcgaagacttctttgtga

SEQ ID NO: 43 - RNA, 19 nt, synthetic construct
  source 1..19: mol_type = other RNA
cagtggtcctgttccagaa

SEQ ID NO: 44 - RNA, 19 nt, synthetic construct
  source 1..19: mol_type = other RNA
ctttgctccatgggcatat

SEQ ID NO: 45 - RNA, 19 nt, synthetic construct
  source 1..19: mol_type = other RNA
gacagcagcaattcttgga

SEQ ID NO: 46 - RNA, 19 nt, synthetic construct
  source 1..19: mol_type = other RNA
caggaagatatcagtccta

SEQ ID NO: 47 - RNA, 19 nt, synthetic construct
  source 1..19: mol_type = other RNA
gaaaatgtttaaggatatt

SEQ ID NO: 48 - RNA, 19 nt, synthetic construct
  source 1..19: mol_type = other RNA
ccaatgtgactgaaaatgt

SEQ ID NO: 49 - RNA, 19 nt, synthetic construct
  source 1..19: mol_type = other RNA
ggaaattgtgtcacaatca

SEQ ID NO: 50 - RNA, 19 nt, synthetic construct
  source 1..19: mol_type = other RNA
ccgttaacaaagtgaaaaa

SEQ ID NO: 51 - RNA, 19 nt, synthetic construct
  source 1..19: mol_type = other RNA
ctgagtatctgcaaaagtt

SEQ ID NO: 52 - RNA, 19 nt, synthetic construct
  source 1..19: mol_type = other RNA
cttcagatctgcaacttta

SEQ ID NO: 53 - RNA, 19 nt, synthetic construct
  source 1..19: mol_type = other RNA
ccatttgttggaagatgtt

SEQ ID NO: 54 - RNA, 19 nt, synthetic construct
  source 1..19: mol_type = other RNA
ccagtatggcattcttaca

SEQ ID NO: 55 - RNA, 19 nt, synthetic construct
  source 1..19: mol_type = other RNA
cattttccttaaaggaata

SEQ ID NO: 56 - RNA, 19 nt, synthetic construct
  source 1..19: mol_type = other RNA
gaatttgccgcttctgttt

SEQ ID NO: 57 - RNA, 19 nt, synthetic construct
  source 1..19: mol_type = other RNA
cattgcttcaggcaataat

SEQ ID NO: 58 - RNA, 19 nt, synthetic construct
  source 1..19: mol_type = other RNA
ccacattcatcagaataca

SEQ ID NO: 59 - RNA, 19 nt, synthetic construct
  source 1..19: mol_type = other RNA
ctatgatcgcattatcgaa

SEQ ID NO: 60 - RNA, 19 nt, synthetic construct
  source 1..19: mol_type = other RNA
gaacattcaaattcacaaa

SEQ ID NO: 61 - RNA, 19 nt, synthetic construct
  source 1..19: mol_type = other RNA
cgttctaggagtggaaaaa

SEQ ID NO: 62 - RNA, 19 nt, synthetic construct
  source 1..19: mol_type = other RNA
gtcaacttgagatggttaa

SEQ ID NO: 63 - RNA, 19 nt, synthetic construct
  source 1..19: mol_type = other RNA
ccgacaaaaacttctagaa

SEQ ID NO: 64 - RNA, 19 nt, synthetic construct
  source 1..19: mol_type = other RNA
cgaaggaggttgattgcaa

SEQ ID NO: 65 - RNA, 19 nt, synthetic construct
  source 1..19: mol_type = other RNA
gtagagacacgtccattaa

SEQ ID NO: 66 - RNA, 19 nt, synthetic construct
  source 1..19: mol_type = other RNA
gttcatagatcccatattg

SEQ ID NO: 67 - RNA, 19 nt, synthetic construct
  source 1..19: mol_type = other RNA
tatcgaaggtcactacaat

SEQ ID NO: 68 - RNA, 19 nt, synthetic construct
  source 1..19: mol_type = other RNA
ggaaagatgttctctatag

SEQ ID NO: 69 - RNA, 19 nt, synthetic construct
  source 1..19: mol_type = other RNA
gataatacctgttggaatc

SEQ ID NO: 70 - RNA, 19 nt, synthetic construct
  source 1..19: mol_type = other RNA
gttcatagatcccatattg

SEQ ID NO: 71 - RNA, 19 nt, synthetic construct
  source 1..19: mol_type = other RNA
agaaagaaatgttgcagta

SEQ ID NO: 72 - RNA, 19 nt, synthetic construct
  source 1..19: mol_type = other RNA
gaggtgttattagaatgtt

SEQ ID NO: 73 - RNA, 19 nt, synthetic construct
  source 1..19: mol_type = other RNA
gtaggaaagtttatccagt

SEQ ID NO: 74 - RNA, 19 nt, synthetic construct
  source 1..19: mol_type = other RNA
catcagtcttcgaactcaa

SEQ ID NO: 75 - RNA, 19 nt, synthetic construct
  source 1..19: mol_type = other RNA
ggcatatagttgaattact

SEQ ID NO: 76 - RNA, 19 nt, synthetic construct
  source 1..19: mol_type = other RNA
ccatcagtcttcgaactca

SEQ ID NO: 77 - RNA, 19 nt, synthetic construct
  source 1..19: mol_type = other RNA
gcagcaatcaaaagccgtt

SEQ ID NO: 78 - RNA, 19 nt, synthetic construct
  source 1..19: mol_type = other RNA
ggtcactacaatggtgaac

SEQ ID NO: 79 - RNA, 19 nt, synthetic construct
  source 1..19: mol_type = other RNA
gctatatgaaaacagacaa

SEQ ID NO: 80 - RNA, 19 nt, synthetic construct
  source 1..19: mol_type = other RNA
ggtgttattagaatgttac

SEQ ID NO: 81 - RNA, 19 nt, synthetic construct
  source 1..19: mol_type = other RNA
gtgttggtcgatgtaagca

SEQ ID NO: 82 - RNA, 19 nt, synthetic construct
  source 1..19: mol_type = other RNA
gcacacaagtgaggaatgg

SEQ ID NO: 83 - RNA, 19 nt, synthetic construct
  source 1..19: mol_type = other RNA
gccgttaacaaagtgaaaa

SEQ ID NO: 84 - RNA, 19 nt, synthetic construct
  source 1..19: mol_type = other RNA
atctattgtaccaagggtc

SEQ ID NO: 85 - RNA, 19 nt, synthetic construct
  source 1..19: mol_type = other RNA
atgttgagattattgcctg

SEQ ID NO: 86 - RNA, 19 nt, synthetic construct
  source 1..19: mol_type = other RNA
agtaattcaactatatgcc

SEQ ID NO: 87 - RNA, 19 nt, synthetic construct
  source 1..19: mol_type = other RNA
taacaacgcttgctccagg

SEQ ID NO: 88 - RNA, 19 nt, synthetic construct
  source 1..19: mol_type = other RNA
taatggacgtgtctctacc

SEQ ID NO: 89 - RNA, 19 nt, synthetic construct
  source 1..19: mol_type = other RNA
atctggtgtttcatcgagc

SEQ ID NO: 90 - RNA, 19 nt, synthetic construct
  source 1..19: mol_type = other RNA
tttgtcggttgcattgagg

SEQ ID NO: 91 - RNA, 19 nt, synthetic construct
  source 1..19: mol_type = other RNA
tcagtctgatcattgccgg

SEQ ID NO: 92 - RNA, 19 nt, synthetic construct
  source 1..19: mol_type = other RNA
ttgtgtgcttacatcgacc

SEQ ID NO: 93 - RNA, 19 nt, synthetic construct
  source 1..19: mol_type = other RNA
agatagtcaatatgggatc

SEQ ID NO: 94 - RNA, 19 nt, synthetic construct
  source 1..19: mol_type = other RNA
aagtaattcaactatatgc

SEQ ID NO: 95 - RNA, 19 nt, synthetic construct
  source 1..19: mol_type = other RNA
taaaaagaacgtaagaaag

SEQ ID NO: 96 - RNA, 19 nt, synthetic construct
  source 1..19: mol_type = other RNA
attgatataaataacattc

SEQ ID NO: 97 - RNA, 19 nt, synthetic construct
  source 1..19: mol_type = other RNA
tattctagaagtttttgtc

SEQ ID NO: 98 - RNA, 19 nt, synthetic construct
  source 1..19: mol_type = other RNA
taaactttcctactgtttc

SEQ ID NO: 99 - RNA, 19 nt, synthetic construct
  source 1..19: mol_type = other RNA
taagaaacatctattgtac

SEQ ID NO: 100 - RNA, 19 nt, synthetic construct
  source 1..19: mol_type = other RNA
tgttaacggcttttgattg

SEQ ID NO: 101 - RNA, 19 nt, synthetic construct
  source 1..19: mol_type = other RNA
ttgatataaataacattcc

SEQ ID NO: 102 - RNA, 19 nt, synthetic construct
  source 1..19: mol_type = other RNA
tctatagagaacatctttc

SEQ ID NO: 103 - RNA, 19 nt, synthetic construct
  source 1..19: mol_type = other RNA
ttgtgacacaatttcccag

SEQ ID NO: 104 - RNA, 19 nt, synthetic construct
  source 1..19: mol_type = other RNA
agaaaggcgtcttgcaagc

SEQ ID NO: 105 - RNA, 19 nt, synthetic construct
  source 1..19: mol_type = other RNA
tgcaagctatgatggcctc

SEQ ID NO: 106 - RNA, 19 nt, synthetic construct
  source 1..19: mol_type = other RNA
tagaacgtgtgccttccag

SEQ ID NO: 107 - RNA, 19 nt, synthetic construct
  source 1..19: mol_type = other RNA
tgtgtggacataatggcac

SEQ ID NO: 108 - RNA, 19 nt, synthetic construct
  source 1..19: mol_type = other RNA
tcacaagacaatatgtggc

SEQ ID NO: 109 - RNA, 19 nt, synthetic construct
  source 1..19: mol_type = other RNA
ttaaccatctcaagttgac

SEQ ID NO: 110 - RNA, 19 nt, synthetic construct
  source 1..19: mol_type = other RNA
agaaggtgataaactgctg

SEQ ID NO: 111 - RNA, 19 nt, synthetic construct
  source 1..19: mol_type = other RNA
agttgcagctttaaccatc

SEQ ID NO: 112 - RNA, 19 nt, synthetic construct
  source 1..19: mol_type = other RNA
tagacagagtatctaccac

SEQ ID NO: 113 - RNA, 19 nt, synthetic construct
  source 1..19: mol_type = other RNA
attattgcctgaagcaatg

SEQ ID NO: 114 - RNA, 19 nt, synthetic construct
  source 1..19: mol_type = other RNA
tcttgctacactccacagg

SEQ ID NO: 115 - RNA, 19 nt, synthetic construct
  source 1..19: mol_type = other RNA
tcctactgtttcatggcag

SEQ ID NO: 116 - RNA, 19 nt, synthetic construct
  source 1..19: mol_type = other RNA
tctggtgtttcatcgagcc

SEQ ID NO: 117 - RNA, 19 nt, synthetic construct
  source 1..19: mol_type = other RNA
tttgcaatcaacctccttc

SEQ ID NO: 118 - RNA, 19 nt, synthetic construct
  source 1..19: mol_type = other RNA
tgtgtgtggacataatggc

SEQ ID NO: 119 - RNA, 19 nt, synthetic construct
  source 1..19: mol_type = other RNA
agtctgatcattgccggtg

SEQ ID NO: 120 - RNA, 19 nt, synthetic construct
  source 1..19: mol_type = other RNA
agcttatggatcaaggtac

SEQ ID NO: 121 - RNA, 19 nt, synthetic construct
  source 1..19: mol_type = other RNA
attgcctcttgtactctac

SEQ ID NO: 122 - RNA, 19 nt, synthetic construct
  source 1..19: mol_type = other RNA
aggactgatatcttcctgg

SEQ ID NO: 123 - RNA, 19 nt, synthetic construct
  source 1..19: mol_type = other RNA
actcattaatggacgtgtc

SEQ ID NO: 124 - RNA, 19 nt, synthetic construct
  source 1..19: mol_type = other RNA
tcacaaagaagtcttcgac

SEQ ID NO: 125 - RNA, 19 nt, synthetic construct
  source 1..19: mol_type = other RNA
ttctggaacaggaccactg

SEQ ID NO: 126 - RNA, 19 nt, synthetic construct
  source 1..19: mol_type = other RNA
atatgcccatggagcaaag

SEQ ID NO: 127 - RNA, 19 nt, synthetic construct
  source 1..19: mol_type = other RNA
tccaagaattgctgctgtc

SEQ ID NO: 128 - RNA, 19 nt, synthetic construct
  source 1..19: mol_type = other RNA
taggactgatatcttcctg

SEQ ID NO: 129 - RNA, 19 nt, synthetic construct
  source 1..19: mol_type = other RNA
aatatccttaaacattttc

SEQ ID NO: 130 - RNA, 19 nt, synthetic construct
  source 1..19: mol_type = other RNA
acattttcagtcacattgg

SEQ ID NO: 131 - RNA, 19 nt, synthetic construct
  source 1..19: mol_type = other RNA
tgattgtgacacaatttcc

SEQ ID NO: 132 - RNA, 19 nt, synthetic construct
  source 1..19: mol_type = other RNA
tttttcactttgttaacgg

SEQ ID NO: 133 - RNA, 19 nt, synthetic construct
  source 1..19: mol_type = other RNA
aacttttgcagatactcag

SEQ ID NO: 134 - RNA, 19 nt, synthetic construct
  source 1..19: mol_type = other RNA
taaagttgcagatctgaag

SEQ ID NO: 135 - RNA, 19 nt, synthetic construct
  source 1..19: mol_type = other RNA
aacatcttccaacaaatgg

SEQ ID NO: 136 - RNA, 19 nt, synthetic construct
  source 1..19: mol_type = other RNA
tgtaagaatgccatactgg

SEQ ID NO: 137 - RNA, 19 nt, synthetic construct
  source 1..19: mol_type = other RNA
tattcctttaaggaaaatg

SEQ ID NO: 138 - RNA, 19 nt, synthetic construct
  source 1..19: mol_type = other RNA
aaacagaagcggcaaattc

SEQ ID NO: 139 - RNA, 19 nt, synthetic construct
  source 1..19: mol_type = other RNA
attattgcctgaagcaatg

SEQ ID NO: 140 - RNA, 19 nt, synthetic construct
  source 1..19: mol_type = other RNA
tgtattctgatgaatgtgg

SEQ ID NO: 141 - RNA, 19 nt, synthetic construct
  source 1..19: mol_type = other RNA
ttcgataatgcgatcatag

SEQ ID NO: 142 - RNA, 19 nt, synthetic construct
  source 1..19: mol_type = other RNA
tttgtgaatttgaatgttc

SEQ ID NO: 143 - RNA, 19 nt, synthetic construct
  source 1..19: mol_type = other RNA
tttttccactcctagaacg

SEQ ID NO: 144 - RNA, 19 nt, synthetic construct
  source 1..19: mol_type = other RNA
ttaaccatctcaagttgac

SEQ ID NO: 145 - RNA, 19 nt, synthetic construct
  source 1..19: mol_type = other RNA
ttctagaagtttttgtcgg

SEQ ID NO: 146 - RNA, 19 nt, synthetic construct
  source 1..19: mol_type = other RNA
ttgcaatcaacctccttcg

SEQ ID NO: 147 - RNA, 19 nt, synthetic construct
  source 1..19: mol_type = other RNA
ttaatggacgtgtctctac

SEQ ID NO: 148 - RNA, 19 nt, synthetic construct
  source 1..19: mol_type = other RNA
caatatgggatctatgaac

SEQ ID NO: 149 - RNA, 19 nt, synthetic construct
  source 1..19: mol_type = other RNA
attgtagtgaccttcgata

SEQ ID NO: 150 - RNA, 19 nt, synthetic construct
  source 1..19: mol_type = other RNA
ctatagagaacatctttcc

SEQ ID NO: 151 - RNA, 19 nt, synthetic construct
  source 1..19: mol_type = other RNA
gattccaacaggtattatc

SEQ ID NO: 152 - RNA, 19 nt, synthetic construct
  source 1..19: mol_type = other RNA
caatatgggatctatgaac

SEQ ID NO: 153 - RNA, 19 nt, synthetic construct
  source 1..19: mol_type = other RNA
tactgcaacatttctttct

SEQ ID NO: 154 - RNA, 19 nt, synthetic construct
  source 1..19: mol_type = other RNA
aacattctaataacacctc

SEQ ID NO: 155 - RNA, 19 nt, synthetic construct
  source 1..19: mol_type = other RNA
actggataaactttcctac

SEQ ID NO: 156 - RNA, 19 nt, synthetic construct
  source 1..19: mol_type = other RNA
ttgagttcgaagactgatg

SEQ ID NO: 157 - RNA, 19 nt, synthetic construct
  source 1..19: mol_type = other RNA
agtaattcaactatatgcc

SEQ ID NO: 158 - RNA, 19 nt, synthetic construct
  source 1..19: mol_type = other RNA
tgagttcgaagactgatgg

SEQ ID NO: 159 - RNA, 19 nt, synthetic construct
  source 1..19: mol_type = other RNA
aacggcttttgattgctgc

SEQ ID NO: 160 - RNA, 19 nt, synthetic construct
  source 1..19: mol_type = other RNA
gttcaccattgtagtgacc

SEQ ID NO: 161 - RNA, 19 nt, synthetic construct
  source 1..19: mol_type = other RNA
ttgtctgttttcatatagc

SEQ ID NO: 162 - RNA, 19 nt, synthetic construct
  source 1..19: mol_type = other RNA
gtaacattctaataacacc

SEQ ID NO: 163 - RNA, 19 nt, synthetic construct
  source 1..19: mol_type = other RNA
tgcttacatcgaccaacac

SEQ ID NO: 164 - RNA, 19 nt, synthetic construct
  source 1..19: mol_type = other RNA
ccattcctcacttgtgtgc

SEQ ID NO: 165 - RNA, 19 nt, synthetic construct
  source 1..19: mol_type = other RNA
ttttcactttgttaacggc

SEQ ID NO: 166 - DNA, 20 bp, synthetic construct
  source 1..20: mol_type = other DNA
agtttctgccggttgcactg

SEQ ID NO: 167 - DNA, 20 bp, synthetic construct
  source 1..20: mol_type = other DNA
gggtcaactcgagatggtca

SEQ ID NO: 168 - DNA, 20 bp, synthetic construct
  source 1..20: mol_type = other DNA
caagagcgagacgtccataa

SEQ ID NO: 169 - DNA, 20 bp, synthetic construct
  source 1..20: mol_type = other DNA
tcgtatgatcgcataatcga

SEQ ID NO: 170 - DNA, 20 bp, synthetic construct
  source 1..20: mol_type = other DNA
aaactacggctacgtccgag

SEQ ID NO: 171 - DNA, 22 bp, synthetic construct
  source 1..22: mol_type = other DNA
ccgcgagtaactgtggtattta

SEQ ID NO: 172 - DNA, 23 bp, synthetic construct
  source 1..23: mol_type = other DNA
ggttgagaaccactgagttaaga

SEQ ID NO: 173 - DNA, 20 bp, synthetic construct
  source 1..20: mol_type = other DNA
atcccatctctgggtttgcg

SEQ ID NO: 174 - DNA, 20 bp, synthetic construct
  source 1..20: mol_type = other DNA
cccttatggacgtctcgctc

SEQ ID NO: 175 - DNA, 20 bp, synthetic construct
  source 1..20: mol_type = other DNA
gaggactgggttgactgtgg

SEQ ID NO: 176 - DNA, 20 bp, synthetic construct
  source 1..20: mol_type = other DNA
ttccgcaaacccagagatgg

SEQ ID NO: 177 - DNA, 20 bp, synthetic construct
  source 1..20: mol_type = other DNA
agggaaagtagagcagacac

SEQ ID NO: 178 - DNA, 20 bp, synthetic construct
  source 1..20: mol_type = other DNA
aaagaagtcttcgaccaatg

SEQ ID NO: 179 - DNA, 20 bp, synthetic construct
  source 1..20: mol_type = other DNA
agcctgtggagtgtagcaag

SEQ ID NO: 180 - DNA, 20 bp, synthetic construct
  source 1..20: mol_type = other DNA
cagatgaatccctacgaagg

SEQ ID NO: 181 - DNA, 20 bp, synthetic construct
  source 1..20: mol_type = other DNA
tgaactggtagaaacagaag

SEQ ID NO: 182 - DNA, 20 bp, synthetic construct
  source 1..20: mol_type = other DNA
caacagatgaatccctacga

SEQ ID NO: 183 - DNA, 20 bp, synthetic construct
  source 1..20: mol_type = other DNA
ctaagaagaatgagagcctg

SEQ ID NO: 184 - DNA, 20 bp, synthetic construct
  source 1..20: mol_type = other DNA
accttgatccataagcttgg

SEQ ID NO: 185 - DNA, 20 bp, synthetic construct
  source 1..20: mol_type = other DNA
tctgaacaagaggggactgg

SEQ ID NO: 186 - DNA, 20 bp, synthetic construct
  source 1..20: mol_type = other DNA
caagtctgccatgaaacagt

SEQ ID NO: 187 - DNA, 20 bp, synthetic construct
  source 1..20: mol_type = other DNA
acttcatgtctttatcatgg

SEQ ID NO: 188 - DNA, 20 bp, synthetic construct
  source 1..20: mol_type = other DNA
gaaggcacacgttctaggag

SEQ ID NO: 189 - DNA, 20 bp, synthetic construct
  source 1..20: mol_type = other DNA
aggctcgatgaaacaccaga

SEQ ID NO: 190 - DNA, 20 bp, synthetic construct
  source 1..20: mol_type = other DNA
tgatgggacagttgtgctgg

SEQ ID NO: 191 - DNA, 20 bp, synthetic construct
  source 1..20: mol_type = other DNA
gatcattgccggtgagacag

SEQ ID NO: 192 - DNA, 20 bp, synthetic construct
  source 1..20: mol_type = other DNA
aaactatggttgtgtccgag

SEQ ID NO: 193 - DNA, 20 bp, synthetic construct
  source 1..20: mol_type = other DNA
ggggcttcttcatacgacga

SEQ ID NO: 194 - DNA, 20 bp, synthetic construct
  source 1..20: mol_type = other DNA
caggcaagccacaatgtgtg

SEQ ID NO: 195 - DNA, 20 bp, synthetic construct
  source 1..20: mol_type = other DNA
tcagccaggagcagctggtg

SEQ ID NO: 196 - DNA, 20 bp, synthetic construct
  source 1..20: mol_type = other DNA
cccacctaacctgatcagcc

SEQ ID NO: 197 - DNA, 20 bp, synthetic construct
  source 1..20: mol_type = other DNA
atggctgtgcaaaatccact

SEQ ID NO: 198 - DNA, 20 bp, synthetic construct
  source 1..20: mol_type = other DNA
tttatgcagttctgaacaag

SEQ ID NO: 199 - DNA, 20 bp, synthetic construct
  source 1..20: mol_type = other DNA
cattatgtccacacacattg

SEQ ID NO: 200 - DNA, 20 bp, synthetic construct
  source 1..20: mol_type = other DNA
caagatgtctgggatgacat

SEQ ID NO: 201 - DNA, 20 bp, synthetic construct
  source 1..20: mol_type = other DNA
ggagagaaggtagcacaggc

SEQ ID NO: 202 - DNA, 20 bp, synthetic construct
  source 1..20: mol_type = other DNA
tcgatgaaacaccagatgga

SEQ ID NO: 203 - DNA, 20 bp, synthetic construct
  source 1..20: mol_type = other DNA
ccagtatggcattcttacag

SEQ ID NO: 204 - DNA, 20 bp, synthetic construct
  source 1..20: mol_type = other DNA
cacctcttgctacactccac

SEQ ID NO: 205 - DNA, 20 bp, synthetic construct
  source 1..20: mol_type = other DNA
tcacaatcacccacactagc

SEQ ID NO: 206 - DNA, 20 bp, synthetic construct
  source 1..20: mol_type = other DNA
cattggagagaaggtagcac

SEQ ID NO: 207 - DNA, 20 bp, synthetic construct
  source 1..20: mol_type = other DNA
gcctgctcctctacagacac

SEQ ID NO: 208 - DNA, 20 bp, synthetic construct
  source 1..20: mol_type = other DNA
gtgtgtggacataatggcac

SEQ ID NO: 209 - DNA, 20 bp, synthetic construct
  source 1..20: mol_type = other DNA
agacttctttgtgatgaaag

SEQ ID NO: 210 - DNA, 20 bp, synthetic construct
  source 1..20: mol_type = other DNA
attatcgaaggtcactacaa

SEQ ID NO: 211 - DNA, 20 bp, synthetic construct
  source 1..20: mol_type = other DNA
aatggttgccactgtctcac

SEQ ID NO: 212 - DNA, 20 bp, synthetic construct
  source 1..20: mol_type = other DNA
tcttgttcagaactgcataa

SEQ ID NO: 213 - DNA, 20 bp, synthetic construct
  source 1..20: mol_type = other DNA
tcttgttcagaactgcataa

SEQ ID NO: 214 - DNA, 20 bp, synthetic construct
  source 1..20: mol_type = other DNA
ggagcagctggtgcggaagg

SEQ ID NO: 215 - DNA, 20 bp, synthetic construct
  source 1..20: mol_type = other DNA
gggaattgatctctccacat

SEQ ID NO: 216 - DNA, 20 bp, synthetic construct
  source 1..20: mol_type = other DNA
ttcttggagatcttcctgga

SEQ ID NO: 217 - DNA, 20 bp, synthetic construct
  source 1..20: mol_type = other DNA
aaagcaccatacattgcttc

SEQ ID NO: 218 - DNA, 20 bp, synthetic construct
  source 1..20: mol_type = other DNA
tcctatgatcgcattatcga

SEQ ID NO: 219 - DNA, 20 bp, synthetic construct
  source 1..20: mol_type = other DNA
tcaatatgggatctatgaac

SEQ ID NO: 220 - DNA, 20 bp, synthetic construct
  source 1..20: mol_type = other DNA
aaaacctcttgtgctcgggc

SEQ ID NO: 221 - DNA, 20 bp, synthetic construct
  source 1..20: mol_type = other DNA
tgctaccttctctccaatga

SEQ ID NO: 222 - DNA, 20 bp, synthetic construct
  source 1..20: mol_type = other DNA
ttattgcctgaagcaatgta

SEQ ID NO: 223 - DNA, 20 bp, synthetic construct
  source 1..20: mol_type = other DNA
tggtgccttcattggagaga

SEQ ID NO: 224 - DNA, 20 bp, synthetic construct
  source 1..20: mol_type = other DNA
gaactcaacttctacagcaa

SEQ ID NO: 225 - DNA, 20 bp, synthetic construct
  source 1..20: mol_type = other DNA
aagtcctgcccgagcacaag

SEQ ID NO: 226 - DNA, 20 bp, synthetic construct
  source 1..20: mol_type = other DNA
accttcgataatgcgatcat

SEQ ID NO: 227 - DNA, 20 bp, synthetic construct
  source 1..20: mol_type = other DNA
ttgcaatcaacctccttcgt

SEQ ID NO: 228 - DNA, 20 bp, synthetic construct
  source 1..20: mol_type = other DNA
gacatcttgataatacctgt

SEQ ID NO: 229 - DNA, 20 bp, synthetic construct
  source 1..20: mol_type = other DNA
ggtgtctgctctactttccc

SEQ ID NO: 230 - DNA, 20 bp, synthetic construct
  source 1..20: mol_type = other DNA
tgtgatgaaagaggaagtcc

SEQ ID NO: 231 - DNA, 20 bp, synthetic construct
  source 1..20: mol_type = other DNA
tcacaaaggtcaacttgaga

SEQ ID NO: 232 - DNA, 20 bp, synthetic construct
  source 1..20: mol_type = other DNA
ctgaatttcctgagaacccc

SEQ ID NO: 233 - DNA, 20 bp, synthetic construct
  source 1..20: mol_type = other DNA
aagttgagttcgaagactga

SEQ ID NO: 234 - DNA, 20 bp, synthetic construct
  source 1..20: mol_type = other DNA
actcatcgttcctgctagtg

SEQ ID NO: 235 - DNA, 20 bp, synthetic construct
  source 1..20: mol_type = other DNA
cgatcataggagattccaac

SEQ ID NO: 236 - DNA, 20 bp, synthetic construct
  source 1..20: mol_type = other DNA
acaatttcccagcagctgta

SEQ ID NO: 237 - DNA, 20 bp, synthetic construct
  source 1..20: mol_type = other DNA
cccagtgatgctgctgatga

SEQ ID NO: 238 - DNA, 20 bp, synthetic construct
  source 1..20: mol_type = other DNA
ctactgtttcatggcagact

SEQ ID NO: 239 - DNA, 20 bp, synthetic construct
  source 1..20: mol_type = other DNA
tggaacattcaaattcacaa

SEQ ID NO: 240 - DNA, 20 bp, synthetic construct
  source 1..20: mol_type = other DNA
ggcagacttggtaaaatgtc

SEQ ID NO: 241 - DNA, 20 bp, synthetic construct
  source 1..20: mol_type = other DNA
caggttaggtggggtgctag

SEQ ID NO: 242 - DNA, 20 bp, synthetic construct
  source 1..20: mol_type = other DNA
tcctggctgatcaggttagg

SEQ ID NO: 243 - DNA, 20 bp, synthetic construct
  source 1..20: mol_type = other DNA
cacaatgtgtgtggacataa

SEQ ID NO: 244 - DNA, 20 bp, synthetic construct
  source 1..20: mol_type = other DNA
gcctgtgtctgtagaggagc

SEQ ID NO: 245 - DNA, 20 bp, synthetic construct
  source 1..20: mol_type = other DNA
ccaggagcagctggtgcgga

SEQ ID NO: 246 - DNA, 20 bp, synthetic construct
  source 1..20: mol_type = other DNA
ccagctgctcctggctgatc

SEQ ID NO: 247 - DNA, 20 bp, synthetic construct
  source 1..20: mol_type = other DNA
ccactgtaagaatgccatac

SEQ ID NO: 248 - DNA, 20 bp, synthetic construct
  source 1..20: mol_type = other DNA
agaacatctttccgtccatc

SEQ ID NO: 249 - DNA, 20 bp, synthetic construct
  source 1..20: mol_type = other DNA
gtaggaaagtttatccagta

SEQ ID NO: 250 - DNA, 20 bp, synthetic construct
  source 1..20: mol_type = other DNA
agttctgaacaagaggggac

SEQ ID NO: 251 - DNA, 20 bp, synthetic construct
  source 1..20: mol_type = other DNA
cctgatcagccaggagcagc

SEQ ID NO: 252 - DNA, 20 bp, synthetic construct
  source 1..20: mol_type = other DNA
agaaagccaaagtcagaaac

SEQ ID NO: 253 - DNA, 20 bp, synthetic construct
  source 1..20: mol_type = other DNA
accttcatcagcagcatcac

SEQ ID NO: 254 - DNA, 20 bp, synthetic construct
  source 1..20: mol_type = other DNA
cagacaccggtttctgactt

SEQ ID NO: 255 - DNA, 20 bp, synthetic construct
  source 1..20: mol_type = other DNA
ccttccgcaccagctgctcc

SEQ ID NO: 256 - DNA, 20 bp, synthetic construct
  source 1..20: mol_type = other DNA
tgctcctggctgatcaggtt

SEQ ID NO: 257 - DNA, 20 bp, synthetic construct
  source 1..20: mol_type = other DNA
tcctggaaggcacacgttct

SEQ ID NO: 258 - DNA, 20 bp, synthetic construct
  source 1..20: mol_type = other DNA
gactgatgggacagttgtgc

SEQ ID NO: 259 - DNA, 20 bp, synthetic construct
  source 1..20: mol_type = other DNA
ctggtaacaacgcttgctcc

SEQ ID NO: 260 - DNA, 20 bp, synthetic construct
  source 1..20: mol_type = other DNA
acaggtattatcaagatgtc

SEQ ID NO: 261 - DNA, 20 bp, synthetic construct
  source 1..20: mol_type = other DNA
ggtacttcatgtctttatca

SEQ ID NO: 262 - DNA, 20 bp, synthetic construct
  source 1..20: mol_type = other DNA
aatgcatgccatacagctgc

SEQ ID NO: 263 - DNA, 20 bp, synthetic construct
  source 1..20: mol_type = other DNA
cagtgagatggtgccttcat

SEQ ID NO: 264 - DNA, 20 bp, synthetic construct
  source 1..20: mol_type = other DNA
aaggaggttgattgcaaatc

SEQ ID NO: 265 - DNA, 20 bp, synthetic construct
  source 1..20: mol_type = other DNA
gcaattcttggagatcttcc

SEQ ID NO: 266 - DNA, 20 bp, synthetic construct
  source 1..20: mol_type = other DNA
tgctgctgggcagaaccatc

SEQ ID NO: 267 - DNA, 20 bp, synthetic construct
  source 1..20: mol_type = other DNA
tgagcagcagatagtcaata

SEQ ID NO: 268 - DNA, 20 bp, synthetic construct
  source 1..20: mol_type = other DNA
acttcgacagcagcaattct

SEQ ID NO: 269 - DNA, 20 bp, synthetic construct
  source 1..20: mol_type = other DNA
taaactttcctactgtttca

SEQ ID NO: 270 - DNA, 20 bp, synthetic construct
  source 1..20: mol_type = other DNA
tcctagaacgtgtgccttcc

SEQ ID NO: 271 - DNA, 20 bp, synthetic construct
  source 1..20: mol_type = other DNA
cacaggctctcattcttctt

SEQ ID NO: 272 - DNA, 20 bp, synthetic construct
  source 1..20: mol_type = other DNA
ctctatagagctttgctcca

SEQ ID NO: 273 - DNA, 20 bp, synthetic construct
  source 1..20: mol_type = other DNA
tgtatggtgctttgatgtta

SEQ ID NO: 274 - DNA, 20 bp, synthetic construct
  source 1..20: mol_type = other DNA
tgcatttctggactcattaa

SEQ ID NO: 275 - DNA, 20 bp, synthetic construct
  source 1..20: mol_type = other DNA
aactgttcaacagcttcttt

SEQ ID NO: 276 - DNA, 20 bp, synthetic construct
  source 1..20: mol_type = other DNA
ggattcatctgttgcatttc

SEQ ID NO: 277 - DNA, 43 bp, synthetic construct
  source 1..43: mol_type = other DNA
cgcttgctccagggaaagtagagcagacaccggcggtttctga

SEQ ID NO: 278 - DNA, 43 bp, synthetic construct
  source 1..43: mol_type = other DNA
ctttcatcacaaagaagtcttcgaccaatgtggtggagagatc

SEQ ID NO: 279 - DNA, 43 bp, synthetic construct
  source 1..43: mol_type = other DNA
gaagaatgagagcctgtggagtgtagcaagaggaggtgttatt

SEQ ID NO: 280 - DNA, 43 bp, synthetic construct
  source 1..43: mol_type = other DNA
agaaatgcaacagatgaatccctacgaaggaggaggttgattg

SEQ ID NO: 281 - DNA, 43 bp, synthetic construct
  source 1..43: mol_type = other DNA
atgggatctatgaactggtagaaacagaagcggcggcaaattc

SEQ ID NO: 282 - DNA, 43 bp, synthetic construct
  source 1..43: mol_type = other DNA
tccagaaatgcaacagatgaatccctacgaaggaggaggttga

SEQ ID NO: 283 - DNA, 43 bp, synthetic construct
  source 1..43: mol_type = other DNA
tagggcaaacctaagaagaatgagagcctgtggtggagtgtag

SEQ ID NO: 284 - DNA, 33 bp, synthetic construct
  source 1..33: mol_type = other DNA
accttgatccataagcttggggggggcttcttc

SEQ ID NO: 285 - DNA, 43 bp, synthetic construct
  source 1..43: mol_type = other DNA
tttatgcagttctgaacaagaggggactgggggggggtcccac

SEQ ID NO: 286 - DNA, 43 bp, synthetic construct
  source 1..43: mol_type = other DNA
gacattttaccaagtctgccatgaaacagtaggaggaaagttt

SEQ ID NO: 287 - DNA, 43 bp, synthetic construct
  source 1..43: mol_type = other DNA
gcaatggggtacttcatgtctttatcatggaggaggccatcat

SEQ ID NO: 288 - DNA, 43 bp, synthetic construct
  source 1..43: mol_type = other DNA
gatcttcctggaaggcacacgttctaggagtggtggaaaaacc

SEQ ID NO: 289 - DNA, 43 bp, synthetic construct
  source 1..43: mol_type = other DNA
catacgacgaaggctcgatgaaacaccagatggtggacggaaa

SEQ ID NO: 290 - DNA, 43 bp, synthetic construct
  source 1..43: mol_type = other DNA
gttcgaagactgatgggacagttgtgctggggggggtgataaa

SEQ ID NO: 291 - DNA, 33 bp, synthetic construct
  source 1..33: mol_type = other DNA
gatcattgccggtgagacagtggtggcaaccat

SEQ ID NO: 292 - DNA, 43 bp, synthetic construct
  source 1..43: mol_type = other DNA
tgttacgaaaaaactatggttgtgtccgagtggtggattttgc

SEQ ID NO: 293 - DNA, 43 bp, synthetic construct
  source 1..43: mol_type = other DNA
cataagcttgggggcttcttcatacgacgaaggaggctcgatg

SEQ ID NO: 294 - DNA, 43 bp, synthetic construct
  source 1..43: mol_type = other DNA
tgtagaggagcaggcaagccacaatgtgtgtggtggacataat

SEQ ID NO: 295 - DNA, 43 bp, synthetic construct
  source 1..43: mol_type = other DNA
cctaacctgatcagccaggagcagctggtgcggcggaaggcgg

SEQ ID NO: 296 - DNA, 43 bp, synthetic construct
  source 1..43: mol_type = other DNA
ccactagcaccccacctaacctgatcagccaggaggagcagct

SEQ ID NO: 297 - DNA, 43 bp, synthetic construct
  source 1..43: mol_type = other DNA
tttaaggaaaatggctgtgcaaaatccactcggcggacacaac

SEQ ID NO: 298 - DNA, 43 bp, synthetic construct
  source 1..43: mol_type = other DNA
gcttgcagcctttatgcagttctgaacaagaggaggggactgg

SEQ ID NO: 299 - DNA, 43 bp, synthetic construct
  source 1..43: mol_type = other DNA
agtcctgtgccattatgtccacacacattgtggtggcttgcct

SEQ ID NO: 300 - DNA, 43 bp, synthetic construct
  source 1..43: mol_type = other DNA
caggtattatcaagatgtctgggatgacattggtggtagacag

SEQ ID NO: 301 - DNA, 43 bp, synthetic construct
  source 1..43: mol_type = other DNA
tgccttcattggagagaaggtagcacaggctggtggccgcctt

SEQ ID NO: 302 - DNA, 43 bp, synthetic construct
  source 1..43: mol_type = other DNA
cgacgaaggctcgatgaaacaccagatggacggcggaaagatg

SEQ ID NO: 303 - DNA, 43 bp, synthetic construct
  source 1..43: mol_type = other DNA
gaaagtttatccagtatggcattcttacagtggtggcagaggt

SEQ ID NO: 304 - DNA, 43 bp, synthetic construct
  source 1..43: mol_type = other DNA
attctaataacacctcttgctacactccacaggaggctctcat

SEQ ID NO: 305 - DNA, 43 bp, synthetic construct
  source 1..43: mol_type = other DNA
ggaaattgtgtcacaatcacccacactagcaggaggaacgatg

SEQ ID NO: 306 - DNA, 43 bp, synthetic construct
  source 1..43: mol_type = other DNA
atggtgccttcattggagagaaggtagcacaggaggctggccg

SEQ ID NO: 307 - DNA, 43 bp, synthetic construct
  source 1..43: mol_type = other DNA
attgtggcttgcctgctcctctacagacacaggaggcaggtat

SEQ ID NO: 308 - DNA, 43 bp, synthetic construct
  source 1..43: mol_type = other DNA
aagccacaatgtgtgtggacataatggcacaggaggacttgct

SEQ ID NO: 309 - DNA, 43 bp, synthetic construct
  source 1..43: mol_type = other DNA
cattggtcgaagacttctttgtgatgaaagaggaggaagtcct

SEQ ID NO: 310 - DNA, 43 bp, synthetic construct
  source 1..43: mol_type = other DNA
ctatgatcgcattatcgaaggtcactacaatggtggtgaacaa

SEQ ID NO: 311 - DNA, 43 bp, synthetic construct
  source 1..43: mol_type = other DNA
ttcttcaagaaatggttgccactgtctcaccggcggcaatgat

SEQ ID NO: 312 - DNA, 43 bp, synthetic construct
  source 1..43: mol_type = other DNA
cccagtcccctcttgttcagaactgcataaaggaggctgcaag

SEQ ID NO: 313 - DNA, 43 bp, synthetic construct
  source 1..43: mol_type = other DNA
cctgagaaccccaggtcaaaatcacgagccaggaggacttcct

SEQ ID NO: 314 - DNA, 43 bp, synthetic construct
  source 1..43: mol_type = other DNA
tgatcagccaggagcagctggtgcggaaggcggcggccagcct

SEQ ID NO: 315 - DNA, 33 bp, synthetic construct
  source 1..33: mol_type = other DNA
gggaattgatctctccacattggtggtcgaaga

SEQ ID NO: 316 - DNA, 43 bp, synthetic construct
  source 1..43: mol_type = other DNA
acagcagcaattcttggagatcttcctggaaggaggcacacgt

SEQ ID NO: 317 - DNA, 43 bp, synthetic construct
  source 1..43: mol_type = other DNA
ccataacatcaaagcaccatacattgcttcaggaggcaataat

SEQ ID NO: 318 - DNA, 43 bp, synthetic construct
  source 1..43: mol_type = other DNA
tgttggaatctcctatgatcgcattatcgaaggaggtcactac

SEQ ID NO: 319 - DNA, 43 bp, synthetic construct
  source 1..43: mol_type = other DNA
cagcagatagtcaatatgggatctatgaactggtggtagaaac

SEQ ID NO: 320 - DNA, 43 bp, synthetic construct
  source 1..43: mol_type = other DNA
taggagtggaaaaacctcttgtgctcgggcaggaggacttttg

SEQ ID NO: 321 - DNA, 43 bp, synthetic construct
  source 1..43: mol_type = other DNA
ggccagcctgtgctaccttctctccaatgaaggaggcaccatc

SEQ ID NO: 322 - DNA, 43 bp, synthetic construct
  source 1..43: mol_type = other DNA
gatgttgagattattgcctgaagcaatgtatggtggtgctttg

SEQ ID NO: 323 - DNA, 43 bp, synthetic construct
  source 1..43: mol_type = other DNA
accagtgagatggtgccttcattggagagaaggaggtagcaca

SEQ ID NO: 324 - DNA, 43 bp, synthetic construct
  source 1..43: mol_type = other DNA
atcagtcttcgaactcaacttctacagcaatggtggggtactt

SEQ ID NO: 325 - DNA, 43 bp, synthetic construct
  source 1..43: mol_type = other DNA
caactgacaaaagtcctgcccgagcacaagaggaggtttttcc

SEQ ID NO: 326 - DNA, 43 bp, synthetic construct
  source 1..43: mol_type = other DNA
cattgtagtgaccttcgataatgcgatcataggaggagattcc

SEQ ID NO: 327 - DNA, 43 bp, synthetic construct
  source 1..43: mol_type = other DNA
tcagccagatttgcaatcaacctccttcgtaggagggattcat

SEQ ID NO: 328 - DNA, 43 bp, synthetic construct
  source 1..43: mol_type = other DNA
tgtcatcccagacatcttgataatacctgttggtggaatctcc

SEQ ID NO: 329 - DNA, 43 bp, synthetic construct
  source 1..43: mol_type = other DNA
gtcagaaaccggtgtctgctctactttccctggtggagcaagc

SEQ ID NO: 330 - DNA, 43 bp, synthetic construct
  source 1..43: mol_type = other DNA
aagacttctttgtgatgaaagaggaagtcctggtggctcgtga

SEQ ID NO: 331 - DNA, 43 bp, synthetic construct
  source 1..43: mol_type = other DNA
acattcaaattcacaaaggtcaacttgagatggtggttaaagc

SEQ ID NO: 332 - DNA, 43 bp, synthetic construct
  source 1..43: mol_type = other DNA
actacatcttctgaatttcctgagaaccccaggaggtcaaaat

SEQ ID NO: 333 - DNA, 43 bp, synthetic construct
  source 1..43: mol_type = other DNA
attgctgtagaagttgagttcgaagactgatggtgggacagtt

SEQ ID NO: 334 - DNA, 43 bp, synthetic construct
  source 1..43: mol_type = other DNA
gtgataaaaaactcatcgttcctgctagtgtggtgggtgattg

SEQ ID NO: 335 - DNA, 43 bp, synthetic construct
  source 1..43: mol_type = other DNA
ttcgataatgcgatcataggagattccaacaggaggtattatc

SEQ ID NO: 336 - DNA, 43 bp, synthetic construct
  source 1..43: mol_type = other DNA
tgattgtgacacaatttcccagcagctgtatggtggcatgcat

SEQ ID NO: 337 - DNA, 33 bp, synthetic construct
  source 1..33: mol_type = other DNA
cccagtgatgctgctgatgaaggaggtagagac

SEQ ID NO: 338 - DNA, 43 bp, synthetic construct
  source 1..43: mol_type = other DNA
ataaactttcctactgtttcatggcagacttggtggtaaaatg

SEQ ID NO: 339 - DNA, 43 bp, synthetic construct
  source 1..43: mol_type = other DNA
cagcttcttttggaacattcaaattcacaaaggaggtcaactt

SEQ ID NO: 340 - DNA, 43 bp, synthetic construct
  source 1..43: mol_type = other DNA
actgtttcatggcagacttggtaaaatgtctggtggcaaggct

SEQ ID NO: 341 - DNA, 43 bp, synthetic construct
  source 1..43: mol_type = other DNA
cctggctgatcaggttaggtggggtgctagtggtgggaccccc

SEQ ID NO: 342 - DNA, 43 bp, synthetic construct
  source 1..43: mol_type = other DNA
caccagctgctcctggctgatcaggttaggtggtggggtgcta

SEQ ID NO: 343 - DNA, 43 bp, synthetic construct
  source 1..43: mol_type = other DNA
gcaggcaagccacaatgtgtgtggacataatggtggcacagga

SEQ ID NO: 344 - DNA, 33 bp, synthetic construct
  source 1..33: mol_type = other DNA
gcctgtgtctgtagaggagcaggaggcaagcca

SEQ ID NO: 345 - DNA, 43 bp, synthetic construct
  source 1..43: mol_type = other DNA
acctgatcagccaggagcagctggtgcggaaggaggcggccag

SEQ ID NO: 346 - DNA, 43 bp, synthetic construct
  source 1..43: mol_type = other DNA
gccttccgcaccagctgctcctggctgatcaggaggttaggtg

SEQ ID NO: 347 - DNA, 43 bp, synthetic construct
  source 1..43: mol_type = other DNA
cttacctctgccactgtaagaatgccatactggtggataaact

SEQ ID NO: 348 - DNA, 43 bp, synthetic construct
  source 1..43: mol_type = other DNA
agctctatagagaacatctttccgtccatctggtggtgtttca

SEQ ID NO: 349 - DNA, 43 bp, synthetic construct
  source 1..43: mol_type = other DNA
ccatgaaacagtaggaaagtttatccagtatggtggcattctt

SEQ ID NO: 350 - DNA, 43 bp, synthetic construct
  source 1..43: mol_type = other DNA
gcctttatgcagttctgaacaagaggggactggtggggggtcc

SEQ ID NO: 351 - DNA, 43 bp, synthetic construct
  source 1..43: mol_type = other DNA
ccccacctaacctgatcagccaggagcagctggtggtgcggaa

SEQ ID NO: 352 - DNA, 43 bp, synthetic construct
  source 1..43: mol_type = other DNA
aggaatatttagaaagccaaagtcagaaaccggcggtgtctgc

SEQ ID NO: 353 - DNA, 43 bp, synthetic construct
  source 1..43: mol_type = other DNA
acgtgtctctaccttcatcagcagcatcactggtgggtctaaa

SEQ ID NO: 354 - DNA, 43 bp, synthetic construct
  source 1..43: mol_type = other DNA
gaaagtagagcagacaccggtttctgactttggtggctttcta

SEQ ID NO: 355 - DNA, 43 bp, synthetic construct
  source 1..43: mol_type = other DNA
aggctggccgccttccgcaccagctgctcctggtggctgatca

SEQ ID NO: 356 - DNA, 43 bp, synthetic construct
  source 1..43: mol_type = other DNA
ccgcaccagctgctcctggctgatcaggttaggaggtggggtg

SEQ ID NO: 357 - DNA, 43 bp, synthetic construct
  source 1..43: mol_type = other DNA
ttggagatcttcctggaaggcacacgttctaggaggagtggaa

SEQ ID NO: 358 - DNA, 43 bp, synthetic construct
  source 1..43: mol_type = other DNA
tgagttcgaagactgatgggacagttgtgctggtgggggtgat

SEQ ID NO: 359 - DNA, 43 bp, synthetic construct
  source 1..43: mol_type = other DNA
ggaagtatagctggtaacaacgcttgctccaggagggaaagta

SEQ ID NO: 360 - DNA, 43 bp, synthetic construct
  source 1..43: mol_type = other DNA
ggagattccaacaggtattatcaagatgtctggtgggatgaca

SEQ ID NO: 361 - DNA, 43 bp, synthetic construct
  source 1..43: mol_type = other DNA
acagcaatggggtacttcatgtctttatcatggtggaggccat

SEQ ID NO: 362 - DNA, 43 bp, synthetic construct
  source 1..43: mol_type = other DNA
aagatgtagtaatgcatgccatacagctgctggtgggaaattg

SEQ ID NO: 363 - DNA, 33 bp, synthetic construct
  source 1..33: mol_type = other DNA
cagtgagatggtgccttcattggtggagagaag

SEQ ID NO: 364 - DNA, 43 bp, synthetic construct
  source 1..43: mol_type = other DNA
aatccctacgaaggaggttgattgcaaatctggtggctgagca

SEQ ID NO: 365 - DNA, 43 bp, synthetic construct
  source 1..43: mol_type = other DNA
ttcgacagcagcaattcttggagatcttcctggtggaaggcac

SEQ ID NO: 366 - DNA, 43 bp, synthetic construct
  source 1..43: mol_type = other DNA
ggcttttgattgctgctgggcagaaccatcaggagggtttaat

SEQ ID NO: 367 - DNA, 43 bp, synthetic construct
  source 1..43: mol_type = other DNA
agaatgaaagtgagcagcagatagtcaatatggtgggatctat

SEQ ID NO: 368 - DNA, 43 bp, synthetic construct
  source 1..43: mol_type = other DNA
tagttgaattacttcgacagcagcaattcttggtggagatctt

SEQ ID NO: 369 - DNA, 43 bp, synthetic construct
  source 1..43: mol_type = other DNA
ccatactggataaactttcctactgtttcatggtggcagactt

SEQ ID NO: 370 - DNA, 43 bp, synthetic construct
  source 1..43: mol_type = other DNA
gtttttccactcctagaacgtgtgccttccaggaggaagatct

SEQ ID NO: 371 - DNA, 43 bp, synthetic construct
  source 1..43: mol_type = other DNA
tgctacactccacaggctctcattcttcttaggaggtttgccc

SEQ ID NO: 372 - DNA, 42 bp, synthetic construct
  source 1..42: mol_type = other DNA
gaaagatgttctctatagagctttgctccatggtggggtatg

SEQ ID NO: 373 - DNA, 43 bp, synthetic construct
  source 1..43: mol_type = other DNA
cctgaagcaatgtatggtgctttgatgttatggtggcagaaga

SEQ ID NO: 374 - DNA, 43 bp, synthetic construct
  source 1..43: mol_type = other DNA
attcatctgttgcatttctggactcattaatggtggacgtgtc

SEQ ID NO: 375 - DNA, 43 bp, synthetic construct
  source 1..43: mol_type = other DNA
gtgctgctaaaactgttcaacagcttcttttggtggaacattc

SEQ ID NO: 376 - DNA, 43 bp, synthetic construct
  source 1..43: mol_type = other DNA
tccttcgtagggattcatctgttgcatttctggtggactcatt

(19) United States
(12) Patent Application Publication
(10) Pub. No.: US 2025/0228885 A1
(43) Pub. Date: Jul. 17, 2025

(54) COMPOSITIONS AND METHODS FOR TREATING LIVER DISEASES WITH SIRNAS TARGETING GPAM

(71) Applicant: The Board of Regents of The University of Texas System, Austin, TX (US)

(72) Inventors: Hao Zhu, Dallas, TX (US); Zixi Wang, Dallas, TX (US); Lin Li, Dallas, TX (US)

(21) Appl. No.: 18/853,813

(22) PCT Filed: Apr. 7, 2023
(86) PCT No.: PCT/US2023/065555

§ 371 Date: Oct. 3, 2024

Related U.S. Application Data
(60) Provisional application No. 63/491,262, filed on Mar. 20, 2023.
(60) Provisional application No. 63/328,593, filed on Apr. 7, 2022.

(51) Int. Cl. A61K 31/713; A61P 1/16; C12N 9/22; C12N 15/11; C12N 15/113; C12N 15/86

(52) U.S. Cl. CPC A61K 31/713; A61P 1/16; C12N 9/22; C12N 15/111; C12N 15/1137; C12N 15/86; C12N 2310/14; C12N 2310/20; C12N 2310/321; C12N 2310/351; C12N 2750/14143

(57) ABSTRACT

Disclosed herein are compositions comprising siRNAs capable of downregulating Glycerol-3-Phosphate Acyltransferase, Mitochondrial (GPAM) gene expression or a variant thereof. Also disclosed herein are methods of using such compositions in the treatment of a liver disease or injury, such as fatty liver disease (FLD), non-alcoholic fatty liver disease (NAFLD) or non-alcoholic steatohepatitis (NASH).

### CROSS REFERENCE TO RELATED APPLICATIONS

This application claims the benefit of the U.S. Provisional Application No. 63/328,593, filed Apr. 7, 2022 and U.S. Provisional Application No. 63/491,262, filed Mar. 20, 2023, the disclosures of which are incorporated herein incorporated by reference in their entireties for all purposes.

### SEQUENCE LISTING

This application contains a Sequence Listing that has been submitted in xml format via EFS-Web and is hereby incorporated by reference in its entirety. The xml copy, created on Apr. 7, 2023, is named 106546-755472.xml and is 329 KB in size.

### BACKGROUND

### 1. Field

The present inventive concept is directed to compositions and methods of use thereof for liver disease treatment, specifically to metabolic liver diseases, including but not limited to non-alcoholic fatty liver disease (NAFLD) and nonalcoholic steatohepatitis (NASH).

### 2. Discussion of Related Art

NAFLD is a spectrum of chronic liver disorders, which encompass, among others nonalcoholic fatty liver (NAFL) and nonalcoholic steatohepatitis (NASH). NASH, a common cause of chronic liver disease, is defined as having at least 5% hepatic steatosis and inflammation with or without fibrosis. Over time and without treatment, NASH may progress to cirrhosis and even hepatocellular carcinoma (HCC). Moreover, NAFLD/NASH is strongly associated with obesity and type II diabetes which together affect over 50% of the US population, leading to a heavy economic burden. Unfortunately, therapeutic options for NASH remain limited, with only slight benefits observed from vitamin E or obeticholic acid treatment. While NASH is the leading cause of chronic liver disease and cirrhosis, there are currently no clinically approved therapies. As such, new targets, therapeutics, and combinations thereof are needed to accelerate clinical progress in the treatment of liver diseases.

### SUMMARY OF THE INVENTION

The present disclosure is based, in part, on the novel finding that positive selection of somatic mutations in NASH patient livers can increase cell fitness and competitiveness through decreased lipid accumulation in NASH. Somatic mutation screening for genes that promote clonal fitness through the reversal of lipotoxicity identified GPAM (Glycerol-3-Phosphate Acyltransferase, Mitochondrial). Accordingly, the present disclosure provides for novel compositions for modulating GPAM expression and methods of preventing, attenuating and/or treating liver diseases.

In some aspects the current disclosure encompasses a composition comprising a nucleic acid molecule that downregulates expression of GPAM or a variant thereof. In some aspects, the nucleic acid is a siRNA, a cluster regularly interspaced short palindromic repeats (CRISPR) related nucleic acid, a single guide RNA (sgRNA), a CRISPR-RNA (crRNA), a trans-activating crRNA (tracrRNA), a plasmid DNA (pDNA), or viral vector. In some aspects the nucleic acid is a small interfering RNA (siRNA) molecule or encodes a siRNA molecule. In some aspects, the composition comprises a plasmid or a viral vector, wherein the plasmid or the viral vector comprises a nucleic acid encoding the siRNA molecule as disclosed herein. In some aspects, the siRNA molecule comprises a nucleotide sequence that is 2 to 30 nucleotides in length and is at least 80% homologous to at least 2 to 30 contiguous nucleotides of a human GPAM cDNA sequence, wherein the human GPAM cDNA sequence is SEQ ID NO: 1. In some aspects, the siRNA molecule targets the open reading frame or the 5′ or 3′ UTRs of the GPAM gene. In some aspects, the siRNA molecule comprises at least one sense sequence, at least one antisense sequence, or at least one sense sequence and at least one antisense sequence. In some aspects, the siRNA molecule comprises a nucleotide sequence of SEQ ID NOs: 2-165, or any combination thereof. In some aspects, the at least one sense sequence comprises SEQ ID NOs: 2-83. In some aspects, the at least one antisense sequence comprises SEQ ID NOs: 84-165.

In some aspects, the current disclosure also encompasses a composition comprising a nucleic acid molecule that downregulates expression of GPAM, wherein the nucleic acid is a sgRNA or encodes an sgRNA. In some aspects, the composition comprises a plasmid or a viral vector, wherein the plasmid or the viral vector comprises a first nucleic acid encoding the sgRNA molecule as disclosed herein and optionally a second nucleic acid encoding an RNA guided nuclease. In some aspects, the sgRNA target sequence is at least about 80% identical to any one of SEQ ID NO: 177-276. In some aspects, the RNA guided nuclease is a Cas endonuclease.

In some aspects, the siRNA molecule as disclosed herein specifically downregulates gene expression of at least one variant of GPAM. In some aspects, the sgRNA molecule as disclosed herein specifically downregulates gene expression of at least one variant of GPAM. In some aspects, the at least one variant of GPAM is associated with a liver disease. Non-limiting examples of liver disease comprises fatty liver disease (FLD), alcohol-related liver disease (ARLD), non-alcoholic fatty liver disease (NAFLD), non-alcoholic steatohepatitis (NASH), end stage liver disease (cirrhosis) from any etiology, liver cancer, or any combination thereof. In some aspects, the at least one variant of GPAM comprises rs10787429 C>T, rs7096937 T>C, rs11446981 T>TA, rs7898213 T>C, rs2792759 C>T, rs2297991 T>C, rs1129555 A>G, rs2254537 T>A, or any combination thereof.

In some aspects, the nucleic acid molecule as disclosed herein may be conjugated to least one targeting ligand. In some aspects, at least one targeting ligand comprises a liver targeting ligand. In some aspects, the liver targeting ligand comprises at least one N-acetylgalactosamine (GalNAc) conjugate. In some aspects, the nucleic acid molecule is conjugated to about one to about three GalNAc conjugates. In some aspects, the nucleic acid molecule comprises at least one chemical modification. In some aspects, the nucleic acid molecule comprises a modification at least one ribosugar moiety of its nucleotide sequence. In some aspects, the at least one ribosugar moiety is modified with 2 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, 2′-C-allyl, or any combination thereof. In some aspects, less than about 10% to about 70% of ribosugar moieties of the total nucleotide sequence is modified.

In some aspects, the current disclosure also encompasses a pharmaceutical composition comprising any one of the compositions as disclosed herein and at least one pharmaceutically acceptable carrier. In some aspects, the pharmaceutical composition further comprises a nanoparticle. In some aspects, the pharmaceutical composition further comprises a lipid.

In some aspects, the current disclosure also encompasses a method of treating a subject in need thereof, the method comprising administrating a therapeutically effective amount of the composition as disclosed herein, or the pharmaceutical composition as disclosed herein. In some aspects, the subject in need thereof, is a human subject having or suspected of having a liver disease. In some aspects, the liver disease comprises fatty liver disease (FLD), alcohol-related liver disease (ARLD), non-alcoholic fatty liver disease (NAFLD), non-alcoholic steatohepatitis (NASH), end stage liver disease (cirrhosis) from any etiology, liver cancer, or any combination thereof. In some aspects, the method of administering comprises parenteral administration. In some aspects, the administration of a therapeutically effective amount of the composition as disclosed here or the pharmaceutical composition as disclosed herein increases life expectancy of the subject compared to an untreated subject with identical disease condition and predicted outcome. In some aspects of the method, administration of a therapeutically effective amount of the composition as disclosed herein or the pharmaceutical composition as disclosed herein increases liver function of the subject compared to an untreated subject with identical disease condition and predicted outcome. In some aspects of the method, administration of a therapeutically effective amount of the composition as disclosed herein or the pharmaceutical composition as disclosed herein attenuates liver fibrosis in the subject compared to an untreated subject with identical disease condition and predicted outcome. In some aspects, the administration of a therapeutically effective amount of the composition as disclosed herein or the pharmaceutical composition as disclosed herein prevents additional liver fibrosis in the subject compared to an untreated subject with identical disease condition and predicted outcome.

In some aspects, the current disclosure also encompasses a kit comprising: a container holding the composition as disclosed here or the pharmaceutical composition as disclosed herein, a pharmaceutical administrative means; and instructions for use.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1A show a screen of 63 known NASH related genes. The scheme for the experiment is shown.

FIG. 1B shows the results of the screen of 63 known NASH related genes. The clones that expanded the most were often associated with deletion of lipogenesis genes such as GPAM.

FIG. 1C provides the top gene hits from the screen, which includes GPAM.

FIG. 1D shows the pathways in which the gene hits are involved. This screen served to show that genes mutated in human cirrhosis tissues are likely to be good drug targets for NASH. GPAM is one such gene that is frequently mutated in human liver disease.

FIG. 2A shows that GPAM mutant clones can out-compete wild-type clones in the liver during NASH development. The red cells are wild-type and were reduced in number over time in the western diet cohort.

FIG. 2B shows red cells that are GPAM mutant and were increased in number over time in the western diet cohort. Furthermore, the NASH phenotype was improved when there were more GPAM mutant clones in the liver (comparing histology between FIG. 2A and FIG. 2B). This experiment proved that GPAM mutant clones were more fit and reduced the level of steatosis in the liver.

FIG. 3A shows a luciferase-based screen used to test the relative knockdown efficacy for various human GPAM siRNAs.

FIG. 3B shows GPAM protein levels examined using western blot analysis after siRNA knockdown.

FIG. 4A shows the genotyping of Gpam^{f/f} liver tissues before and after Cre recombination. The asterisk is next to a hybrid annealing band from the top and bottom PCR products.

FIG. 4B shows qPCR examination of Gpam mRNA levels in livers before and after Cre recombination. Primer design is shown on the left. Two pairs of primers were used (n=3 mice for each group).

FIG. 4C shows the quantification of Tomato+ cells from LSL-tdTomato het liver sections (n=7, 11, 11 mice for each group). Each dot represents one image field; two fields from each mouse liver were analyzed. Statistical analysis in were performed on averaged image data from individual mice.

FIG. 4D shows quantification of Tomato+ cells from Gpam^{f/f}; LSL-tdTomato het liver sections (n=7, 15, 15 mice for each group). Each dot represents one image field; two fields from each mouse liver are shown. Statistical analysis in were performed on averaged image data from individual mice.

FIG. 4E shows body weights of Gpam KO mice treated with chow or WD and the corresponding control mice (n=6, 16, 6, 12 mice for each group).

FIG. 4F shows liver weights of whole-liver Gpam KO mice treated with chow or WD and the corresponding control mice (n=6, 16, 6, 12 mice for each group).

FIG. 4G shows liver/body weight ratios of liver-specific Gpam WT and KO mice fed with 3 months of WD (n=6, 16, 6, 12 mice for each group). These mice were given high doses of AAV8-TBG-Cre to generate liver-wide Gpam deletion in almost all hepatocytes

FIG. 4H shows representative H&E staining of Gpam WT and KO liver sections after 3 months of WD.

FIG. 4I shows liver function testing with plasma ALT (n=6, 16, 6, 12 mice for each group).

FIG. 4J shows liver function testing with plasma AST (n=6, 16, 6, 12 mice for each group).

FIG. 4K shows NAFLD activity score of the H&E sections. The right panel represents the total NAFLD activity score, which is the sum of the three scores on the left (n=13 and 14 mice for each group).

FIG. 4L shows triglyceride measurements from liver tissues in WT and KO mice (n=6, 16, 6, 12 for each group).

FIG. 4M shows cholesterol measurements from liver tissues in WT and KO mice(n=6, 16, 6, 12 for each group).

FIG. 4N shows plasma cholesterol for the WT and KO mice (n=6, 16, 6, 12 for each group).

FIG. 4O shows plasma triglycerides for the WT and KO mice (n=6, 16, 6, 12 for each group).

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

With increasing over-nutrition and obesity, non-alcoholic fatty liver disease (NAFLD) is rapidly becoming the leading cause of liver disease in the world. NAFLD is usually conceptualized at the organismal and tissue levels; however, little thought has been given to genetic heterogeneity within clones of the liver. Somatic mutations are common in most healthy individuals, and there is accumulating evidence that mutation burden increases with age and chronic tissue damage. The present disclosure is based, in part, on the novel finding that positive selection of somatic mutations in NASH patient livers can increase cell fitness and competitiveness through decreased lipid accumulation in NASH. In vivo genetic screening of somatically mutated genes in the liver identified additional genes that when mutated, promoted clonal fitness through the reversal of lipotoxicity, including GPAM (Glycerol-3-Phosphate Acyltransferase, Mitochondrial). Accordingly, provided herein are compositions and methods of use thereof for liver disease treatment, specifically to metabolic liver diseases, including but not limited to non-alcoholic fatty liver disease (NAFLD) and nonalcoholic steatohepatitis (NASH).

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

As used herein, the term “about,” can mean relative to the recited value, e.g., amount, dose, temperature, time, percentage, etc., 10%, 9%, 8%, 7%, 6%, 5%, 4%, 3%, 2%, or ±1%.

The terms “comprising,” “including,” “encompassing” and “having” are used interchangeably in this disclosure. The terms “comprising,” “including,” “encompassing” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

“Biomarker” as used herein refers to any biological molecules (e.g., nucleic acids, genes, peptides, proteins, lipids, hormones, metabolites, and the like) that, singularly or collectively, reflect the current or predict future state of a biological system. Thus, as used herein, the presence or concentration of one or more biomarkers can be detected and correlated with a known condition, such as a disease state. In some aspects, detecting the presence and/or concentration of one or more biomarkers herein may be an indication of a liver disease risk in a subject. In some other aspects, detecting the presence and/or concentration of one or more biomarkers herein may be used in treating and/or preventing a chronic liver disease in a subject.

As used herein, the terms “treat”, “treating”, “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder.

The term “biomolecule” as used herein refers to, but is not limited to, proteins, enzymes, antibodies, DNA, siRNA, and small molecules. “Small molecules” as used herein can refer to chemicals, compounds, drugs, and the like.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

It should also be understood that, unless clearly indicated to the contrary, in any methods claimed herein that include more than one step or act, the order of the steps or acts of the method is not necessarily limited to the order in which the steps or acts of the method are recited.

### II. Compositions

### (a) GPAM

In certain embodiments, compositions for use in the methods disclosed herein can modulate GPAM (Glycerol-3-Phosphate Acyltransferase, Mitochondrial) gene. The GPAM gene encodes a mitochondrial enzyme, Gpam, which catalyzes the acylation of glycerol-3-phosphate with acyl-coenzyme A (CoA) generating CoA and lysophosphatidic acid (LPA). This is the rate-limiting step in TAG synthesis. Two forms for this enzyme exist, one in the mitochondria and one in the endoplasmic reticulum. Additionally, two alternatively spliced transcript variants have been described for the GPAM gene.

As used herein, compositions “modulating” GPAM can include any biomolecule(s) capable of decreasing GPAM gene expression, decreasing GPAM protein expression, decreasing GPAM activity, or a combination thereof. In some aspects, biomolecule(s) herein capable of modulating GPAM can be an inhibitor of GPAM. As used herein, an inhibitor of GPAM can inhibit GPAM direct activity, inhibit GPAM indirect activity, decrease expression of the GPAM gene, decrease expression of the GPAM protein, or a combination thereof.

In certain embodiments, compositions for use in the methods disclosed herein can include a nucleic acid molecule. The term “nucleic acid molecule” as used herein refers to a molecule having nucleotides. The nucleic acid can be single, double, or multiple stranded and may comprise modified or unmodified nucleotides or non-nucleotides or various mixtures and combinations thereof. In some embodiments, a nucleic acid molecule for use herein can be a double-stranded RNA. In some examples, a double stranded RNA suitable for use herein can be small temporal RNA, small nuclear RNA, small nucleolar RNA, short hairpin RNA, microRNA, or the like. In certain embodiments, a double stranded RNA suitable for use herein can be a small interfering RNA (siRNA).

The term “siRNA” as used herein refers to small inhibitory RNA duplexes that induce the RNA interference (RNAi) pathway. A siRNA molecule disclosed herein may be capable of silencing, reducing, and/or inhibiting expression of a target gene (e.g., GPAM). These molecules can vary in length (generally about 5-50 base pairs) and contain varying degrees of complementarity to their target mRNA in the antisense strand. Some, but not all, siRNA have unpaired overhanging bases on the 5′ or 3′ end of the sense strand and/or the antisense strand. The term “siRNA” as used herein can includes duplexes of two separate strands, as well as single strands that can form hairpin structures comprising a duplex region.

In certain embodiments, siRNA molecules disclosed herein may be any interfering RNA with a duplex length of about 2-60, about 5-50, or about 10-40 nucleotides in length, more typically about 2-30, about 5-25, or about 10-25 nucleotides in length. In some embodiments, siRNA molecule disclosed herein may have a nucleotide sequence that is about 2 to about 30 nucleotides in length, about 5 to about 25 nucleotides in length, or about 10 to about 20 nucleotides in length. In some embodiments, siRNA molecule disclosed herein may have a nucleotide sequence that is about 10 nucleotides in length, about 11 nucleotides in length, about 12 nucleotides in length, about 13 nucleotides in length, about 14 nucleotides in length, about 15 nucleotides in length, about 14 nucleotides in length, about 15 nucleotides in length, about 16 nucleotides in length, about 17 nucleotides in length, about 18 nucleotides in length, about 19 nucleotides in length, about 20 nucleotides in length, about 21 nucleotides in length, about 22 nucleotides in length, about 23 nucleotides in length, about 24 nucleotides in length, or about 25 nucleotides in length. Each complementary sequence of the double-stranded siRNA may be about 2-60, about 5-50, about 10-40, about 2-30, about 5-25, or about 10-25 nucleotides in length, but other noncomplementary sequences may be present. For example, siRNA duplexes may comprise 3′ overhangs of about 1 to about 4 or more nucleotides and/or 5′ phosphate termini comprising about 1 to about 4 or more nucleotides.

In certain embodiments, siRNA molecules disclosed herein may have about 2-60, about 2-50, about 2-40, or about 2-30 contiguous nucleotides of homology with a target (e.g., GPAM) nucleotide sequence. In some aspects, a target nucleotide sequence herein may be a human GPAM nucleotide sequence or a variant thereof. In some other aspects, a target nucleotide sequence herein may be human GPAM, RefSeq: NM_001244949.2 or a variant thereof. In still some other aspects, a target nucleotide sequence herein may be SEQ ID NO: 1 as follows, or a variant thereof:

Human GPAM
(SEQ ID NO: 1)
GATGAATCTGCACTGACCCTTGGTACAATAGATGTTTCTTATCTGCCACATTCATCAGAATACAG
TGTTGGTCGATGTAAGCACACAAGTGAGGAATGGGGTGAGTGTGGCTTTAGACCCACCATCTTCA
GATCTGCAACTTTAAAATGGAAAGAAAGCCTAATGAGTCGGAAAAGGCCATTTGTTGGAAGATGT
TGTTACTCCTGCACTCCCCAGAGCTGGGACAAATTTTTCAACCCCAGTATCCCGTCTTTGGGTTT
GCGGAATGTTATTTATATCAATGAAACTCACACAAGACACCGCGGATGGCTTGCAAGACGCCTTT
CTTACGTTCTTTTTATTCAAGAGCGAGATGTGCATAAGGGCATGTTTGCCACCAATGTGACTGAA
AATGTGCTGAACAGCAGTAGAGTACAAGAGGCAATTGCAGAAGTGGCTGCTGAATTAAACCCTGA
TGGTTCTGCCCAGCAGCAATCAAAAGCCGTTAACAAAGTGAAAAAGAAAGCTAAAAGGATTCTTC
AAGAAATGGTTGCCACTGTCTCACCGGCAATGATCAGACTGACTGGGTGGGTGCTGCTAAAACTG
TTCAACAGCTTCTTTTGGAACATTCAAATTCACAAAGGTCAACTTGAGATGGTTAAAGCTGCAAC
TGAGACGAATTTGCCGCTTCTGTTTCTACCAGTTCATAGATCCCATATTGACTATCTGCTGCTCA
CTTTCATTCTCTTCTGCCATAACATCAAAGCACCATACATTGCTTCAGGCAATAATCTCAACATC
CCAATCTTCAGTACCTTGATCCATAAGCTTGGGGGCTTCTTCATACGACGAAGGCTCGATGAAAC
ACCAGATGGACGGAAAGATGTTCTCTATAGAGCTTTGCTCCATGGGCATATAGTTGAATTACTTC
GACAGCAGCAATTCTTGGAGATCTTCCTGGAAGGCACACGTTCTAGGAGTGGAAAAACCTCTTGT
GCTCGGGCAGGACTTTTGTCAGTTGTGGTAGATACTCTGTCTACCAATGTCATCCCAGACATCTT
GATAATACCTGTTGGAATCTCCTATGATCGCATTATCGAAGGTCACTACAATGGTGAACAACTGG
GCAAACCTAAGAAGAATGAGAGCCTGTGGAGTGTAGCAAGAGGTGTTATTAGAATGTTACGAAAA
AACTATGGTTGTGTCCGAGTGGATTTTGCACAGCCATTTTCCTTAAAGGAATATTTAGAAAGCCA
AAGTCAGAAACCGGTGTCTGCTCTACTTTCCCTGGAGCAAGCGTTGTTACCAGCTATACTTCCTT
CAAGACCCAGTGATGCTGCTGATGAAGGTAGAGACACGTCCATTAATGAGTCCAGAAATGCAACA
GATGAATCCCTACGAAGGAGGTTGATTGCAAATCTGGCTGAGCATATTCTATTCACTGCTAGCAA
GTCCTGTGCCATTATGTCCACACACATTGTGGCTTGCCTGCTCCTCTACAGACACAGGCAGGGAA
TTGATCTCTCCACATTGGTCGAAGACTTCTTTGTGATGAAAGAGGAAGTCCTGGCTCGTGATTTT
GACCTGGGGTTCTCAGGAAATTCAGAAGATGTAGTAATGCATGCCATACAGCTGCTGGGAAATTG
TGTCACAATCACCCACACTAGCAGGAACGATGAGTTTTTTATCACCCCCAGCACAACTGTCCCAT
CAGTCTTCGAACTCAACTTCTACAGCAATGGGGTACTTCATGTCTTTATCATGGAGGCCATCATA
GCTTGCAGCCTTTATGCAGTTCTGAACAAGAGGGGACTGGGGGGTCCCACTAGCACCCCACCTAA
CCTGATCAGCCAGGAGCAGCTGGTGCGGAAGGCGGCCAGCCTGTGCTACCTTCTCTCCAATGAAG
GCACCATCTCACTGCCTTGCCAGACATTTTACCAAGTCTGCCATGAAACAGTAGGAAAGTTTATC
CAGTATGGCATTCTTACAGTGGCAGAGCACGATGACCAGGAAGATATCAGTCCTAGTCTTGCTGA
GCAGCAGTGGGACAAGAAGCTTCCAGAACCTTTGTCTTGGAGAAGTGATGAAGAAGATGAAGACA
GTGACTTTGGGGAGGAACAGCGAGATTGCTACCTGAAGGTGAGCCAATCCAAGGAGCACCAGCAG
TTTATCACCTTCTTACAGAGACTCCTTGGGCCTTTGCTGGAGGCCTACAGCTCTGCTGCCATCTT
TGTTCACAACTTCAGTGGTCCTGTTCCAGAACCTGAGTATCTGCAAAAGTTGCACAAATACCTAA
TAACCAGAACAGAAAGAAATGTTGCAGTATATGCTGAGAGTGCCACATATTGTCTTGTGAAGAAT
GCTGTGAAAATGTTTAAGGATATTGGGGTTTTCAAGGAGACCAAACAAAAGAGAGTGTCTGTTTT
AGAACTGAGCAGCACTTTTCTACCTCAATGCAACCGACAAAAACTTCTAGAATATATTCTGAGTT
TTGTGGTGCTGTAG

In certain embodiments, siRNA molecules disclosed herein may have a nucleotide sequence that is about 2-30 contiguous in length and is at least about 80% homologous (e.g., about 80%, about 85%, about 90%, about 95%, or about 99%) to at least about 2 to about 30 contiguous nucleotides of a human GPAM cDNA sequence. In some embodiments, siRNA molecules disclosed herein may have a nucleotide sequence that is about 2-30 contiguous in length and is at least 80% homologous (e.g., about 80%, about 85%, about 90%, about 95%, or about 99%) to at least about 2 to about 30 contiguous nucleotides of the human GPAM sequence RefSeq: NM_001244949.2 or a variant thereof. In some embodiments, siRNA molecules disclosed herein may have a nucleotide sequence that is about 2-30 contiguous in length and is at least 80% homologous (e.g., about 80%, about 85%, about 90%, about 95%, or about 99%) to at least about 2 to about 30 contiguous nucleotides of SEQ ID NO: 1. In some other embodiments, siRNA molecules disclosed herein may have a nucleotide sequence that is about 2-30 contiguous in length and is homologous to at least about 2 to about 30 contiguous nucleotides of SEQ ID NO: 1.

A siRNA molecule disclosed herein may be synthesized in any of a number of conformations. One skilled in the art would recognize the type of siRNA conformation to be used for a particular purpose. Examples of siRNA conformations include, but need not be limited to, a double-stranded polynucleotide molecule assembled from two separate stranded molecules, wherein one strand is the sense strand and the other is the complementary antisense strand; a double-stranded polynucleotide molecule assembled from a single-stranded molecule, where the sense and antisense regions are linked by a nucleic acid-based or non-nucleic acid-based linker; a double-stranded polynucleotide molecule with a hairpin secondary structure having complementary sense and antisense regions; or a circular single-stranded polynucleotide molecule with two or more loop structures and a stem having self-complementary sense and antisense regions. In the case of the circular polynucleotide, the polynucleotide may be processed either in vivo or in vitro to generate an active double-stranded siRNA molecule.

In certain embodiments, siRNA molecules disclosed herein may be double stranded siRNA molecules. In some aspects, double stranded siRNA molecules disclosed herein may have at least one sense sequence. In some other aspects, double stranded siRNA molecules disclosed herein may have at least one antisense sequence. In still some other aspects, double stranded siRNA molecules disclosed herein may have at least one antisense sequence and at least one sense sequence. In some embodiments, double stranded siRNA molecules disclosed herein may have at least one antisense sequence selected from Table 1, at least one sense sequence selected from Table 1, or both.

TABLE 1

| | Sense Sequence | Over- | Antisense Sequence | Over- |
|---|---|---|---|---|
| siRNA ID | (5′-3′) | hang | (5′-3′) | hang |
| GPAM 16 | GACCCUUGGUACAAUAGAU | UU | AUCUAUUGUACCAAGGGUC | UU |
| | (SEQ ID NO: 2) | | (SEQ ID NO: 84) | |
| GPAM 762 | CAGGCAAUAAUCUCAACAU | UU | AUGUUGAGAUUAUUGCCU | UU |
| | (SEQ ID NO: 3) | | G (SEQ ID NO: 85) | |
| GPAM 891 | GGCAUAUAGUUGAAUUACU | UU | AGUAAUUCAACUAUAUGCC | UU |
| | (SEQ ID NO: 4) | | (SEQ ID NO: 86) | |
| GPAM 1267 | CCUGGAGCAAGCGUUGUUA | UU | UAACAACGCUUGCUCCAGG | UU |
| | (SEQ ID NO: 5) | | (SEQ ID NO: 87) | |
| GPAM 1328 | GGUAGAGACACGUCCAUUA | UU | UAAUGGACGUGUCUCUAC | UU |
| | (SEQ ID NO: 6) | | C (SEQ ID NO: 88) | |
| GPAM 835 | GCUCGAUGAAACACCAGAU | UU | AUCUGGUGUUUCAUCGAG | UU |
| | (SEQ ID NO: 7) | | C (SEQ ID NO: 89) | |
| GPAM 2429 | CCUCAAUGCAACCGACAAA | UU | UUUGUCGGUUGCAUUGAG | UU |
| | (SEQ ID NO: 8) | | G (SEQ ID NO: 90) | |
| GPAM 545 | CCGGCAAUGAUCAGACUGA | UU | UCAGUCUGAUCAUUGCCG | UU |
| | (SEQ ID NO: 9) | | G (SEQ ID NO: 91) | |
| GPAM 71 | GGUCGAUGUAAGCACACAA | UU | UUGUGUGCUUACAUCGAC | UU |
| | (SEQ ID NO: 10) | | C (SEQ ID NO: 92) | |
| GPAM 690 | GAUCCCAUAUUGACUAUCU | UU | AGAUAGUCAAUAUGGGAUC | UU |
| | (SEQ ID NO: 11) | | (SEQ ID NO: 93) | |
| GPAM 892-914 | GCAUAUAGUUGAAUUACUU | CG | AAGUAAUUCAACUAUAUGC | CC |
| | (SEQ ID NO: 12) | | (SEQ ID NO: 94) | |
| GPAM 323-345 | CUUUCUUACGUUCUUUUUA | UU | UAAAAAGAACGUAAGAAAG | UU |
| | (SEQ ID NO: 13) | | (SEQ ID NO: 95) | |
| GPAM 265-287 | GAAUGUUAUUUAUAUCAAU | GA | AUUGAUAUAAAUAACAUUC | CG |
| | (SEQ ID NO: 14) | | (SEQ ID NO: 96) | |
| GPAM 2442- | GACAAAAACUUCUAGAAUA | UU | UAUUCUAGAAGUUUUUGUC | UU |
| 2464 | (SEQ ID NO: 15) | | (SEQ ID NO: 97) | |
| GPAM 1931- | GAAACAGUAGGAAAGUUUA | UC | UAAACUUUCCUACUGUUUC | AU |
| 1953 | (SEQ ID NO: 16) | | (SEQ ID NO: 98) | |
| GPAM 24-46 | GUACAAUAGAUGUUUCUUA | UC | UAAGAAACAUCUAUUGUAC | CA |
| | (SEQ ID NO: 17) | | (SEQ ID NO: 99) | |
| GPAM 473-495 | CAAUCAAAAGCCGUUAACA | AA | UGUUAACGGCUUUUGAUU | CU |
| | (SEQ ID NO: 18) | | G (SEQ ID NO: 100) | |
| GPAM 264-286 | GGAAUGUUAUUUAUAUCAA | UG | UUGAUAUAAAUAACAUUCC | UU |
| | (SEQ ID NO: 19) | | (SEQ ID NO: 101) | |
| GPAM 859-881 | GAAAGAUGUUCUCUAUAGA | UU | UCUAUAGAGAACAUCUUUC | CG |
| | (SEQ ID NO: 20) | | (SEQ ID NO: 102) | |
| GPAM 1616 | CUGGGAAAUUGUGUCACAA | UU | UUGUGACACAAUUUCCCAG | UU |
| | (SEQ ID NO: 21) | | (SEQ ID NO: 103) | |
| GPAM 310 | GCUUGCAAGACGCCUUUCU | UU | AGAAAGGCGUCUUGCAAG | UU |
| | (SEQ ID NO: 22) | | C (SEQ ID NO: 104) | |
| GPAM 1745 | GAGGCCAUCAUAGCUUGCA | UU | UGCAAGCUAUGAUGGCCU | UU |
| | (SEQ ID NO: 23) | | C (SEQ ID NO: 105) | |
| GPAM 938 | CUGGAAGGCACACGUUCUA | UU | UAGAACGUGUGCCUUCCA | UU |
| | (SEQ ID NO: 24) | | G (SEQ ID NO: 106) | |
| GPAM 1437 | GUGCCAUUAUGUCCACACA | UU | UGUGUGGACAUAAUGGCA | UU |
| | (SEQ ID NO: 25) | | C (SEQ ID NO: 107) | |
| GPAM 2318 | GCCACAUAUUGUCUUGUGA | UU | UCACAAGACAAUAUGUGGC | UU |
| | (SEQ ID NO: 26) | | (SEQ ID NO: 108) | |
| GPAM 624 | GUCAACUUGAGAUGGUUAA | UU | UUAACCAUCUCAAGUUGAC | UU |
| | (SEQ ID NO: 27) | | (SEQ ID NO: 109) | |
| GPAM 2141 | CAGCAGUUUAUCACCUUCU | UU | AGAAGGUGAUAAACUGCUG | UU |
| | (SEQ ID NO: 28) | | (SEQ ID NO: 110) | |
| GPAM 634 | GAUGGUUAAAGCUGCAACU | UU | AGUUGCAGCUUUAACCAUC | UU |
| | (SEQ ID NO: 29) | | (SEQ ID NO: 111) | |
| GPAM 1001 | GUGGUAGAUACUCUGUCUA | UU | UAGACAGAGUAUCUACCAC | UU |
| | (SEQ ID NO: 30) | | (SEQ ID NO: 112) | |
| GPAM 754 | CAUUGCUUCAGGCAAUAAU | UU | AUUAUUGCCUGAAGCAAUG | UU |
| | (SEQ ID NO: 31) | | (SEQ ID NO: 113) | |
| GPAM 1129 | CCUGUGGAGUGUAGCAAGA | UU | UCUUGCUACACUCCACAGG | UU |
| | (SEQ ID NO: 32) | | (SEQ ID NO: 114) | |
| GPAM 1924 | CUGCCAUGAAACAGUAGGA | UU | UCCUACUGUUUCAUGGCA | UU |
| | (SEQ ID NO: 33) | | G (SEQ ID NO: 115) | |
| GPAM 834 | GGCUCGAUGAAACACCAGA | UU | UCUGGUGUUUCAUCGAGC | UU |
| | (SEQ ID NO: 34) | | C (SEQ ID NO: 116) | |
| GPAM 1380 | GAAGGAGGUUGAUUGCAAA | UU | UUUGCAAUCAACCUCCUUC | UU |
| | (SEQ ID NO: 35) | | (SEQ ID NO: 117) | |
| GPAM 1439 | GCCAUUAUGUCCACACACA | UU | UGUGUGUGGACAUAAUGG | UU |
| | (SEQ ID NO: 36) | | C (SEQ ID NO: 118) | |
| GPAM 543 | CACCGGCAAUGAUCAGACU | UU | AGUCUGAUCAUUGCCGGU | UU |
| | (SEQ ID NO: 37) | | G (SEQ ID NO: 119) | |
| GPAM 792 | GUACCUUGAUCCAUAAGCU | UU | AGCUUAUGGAUCAAGGUAC | UU |
| | (SEQ ID NO: 38) | | (SEQ ID NO: 120) | |
| GPAM 408 | GUAGAGUACAAGAGGCAAU | UU | AUUGCCUCUUGUACUCUAC | UU |
| | (SEQ ID NO: 39) | | (SEQ ID NO: 121) | |
| GPAM 1987 | CCAGGAAGAUAUCAGUCCU | UU | AGGACUGAUAUCUUCCUG | UU |
| | (SEQ ID NO: 40) | | G (SEQ ID NO: 122) | |
| GPAM 1334 | GACACGUCCAUUAAUGAGU | UU | ACUCAUUAAUGGACGUGUC | UU |
| | (SEQ ID NO: 41) | | (SEQ ID NO: 123) | |
| GPAM 1514 | GUCGAAGACUUCUUUGUGA | UU | UCACAAAGAAGUCUUCGAC | UU |
| | (SEQ ID NO: 42) | | (SEQ ID NO: 124) | |
| GPAM 2224 | CAGUGGUCCUGUUCCAGAA | UU | UUCUGGAACAGGACCACU | UU |
| | (SEQ ID NO: 43) | | G (SEQ ID NO: 125) | |
| GPAM 879 | CUUUGCUCCAUGGGCAUAU | UU | AUAUGCCCAUGGAGCAAAG | UU |
| | (SEQ ID NO: 44) | | (SEQ ID NO: 126) | |
| GPAM 912 | GACAGCAGCAAUUCUUGGA | UU | UCCAAGAAUUGCUGCUGU | UU |
| | (SEQ ID NO: 45) | | C (SEQ ID NO: 127) | |
| GPAM 1988 | CAGGAAGAUAUCAGUCCUA | UU | UAGGACUGAUAUCUUCCU | UU |
| | (SEQ ID NO: 46) | | G (SEQ ID NO: 128) | |
| GPAM 2347- | GAAAAUGUUUAAGGAUAUU | GG | AAUAUCCUUAAACAUUUUC | AC |
| 2369 | (SEQ ID NO: 47) | | (SEQ ID NO: 129) | |
| GPAM 378-400 | CCAAUGUGACUGAAAAUGU | GC | ACAUUUUCAGUCACAUUGG | UG |
| | (SEQ ID NO: 48) | | (SEQ ID NO: 130) | |
| GPAM 1619- | GGAAAUUGUGUCACAAUCA | CC | UGAUUGUGACACAAUUUCC | CA |
| 1641 | (SEQ ID NO: 49) | | (SEQ ID NO: 131) | |
| GPAM 483-505 | CCGUUAACAAAGUGAAAAA | GA | UUUUUCACUUUGUUAACGG | CU |
| | (SEQ ID NO: 50) | | (SEQ ID NO: 132) | |
| GPAM 2244- | CUGAGUAUCUGCAAAAGUU | GC | AACUUUUGCAGAUACUCAG | GU |
| 2266 | (SEQ ID NO: 51) | | (SEQ ID NO: 133) | |
| GPAM 127-149 | CUUCAGAUCUGCAACUUUA | AA | UAAAGUUGCAGAUCUGAAG | AU |
| | (SEQ ID NO: 52) | | (SEQ ID NO: 134) | |
| GPAM 179-201 | CCAUUUGUUGGAAGAUGUU | GU | AACAUCUUCCAACAAAUGG | CC |
| | (SEQ ID NO: 53) | | (SEQ ID NO: 135) | |
| GPAM 1951- | CCAGUAUGGCAUUCUUACA | GU | UGUAAGAAUGCCAUACUGG | AU |
| 1973 | (SEQ ID NO: 54) | | (SEQ ID NO: 136) | |
| GPAM 1206- | CAUUUUCCUUAAAGGAAUA | UU | UAUUCCUUUAAGGAAAAUG | GC |
| 1228 | (SEQ ID NO: 55) | | (SEQ ID NO: 137) | |
| GPAM 658-680 | GAAUUUGCCGCUUCUGUUU | CU | AAACAGAAGCGGCAAAUUC | GU |
| | (SEQ ID NO: 56) | | (SEQ ID NO: 138) | |
| GPAM 754-776 | CAUUGCUUCAGGCAAUAAU | CU | AUUAUUGCCUGAAGCAAUG | UA |
| | (SEQ ID NO: 57) | | (SEQ ID NO: 139) | |
| GPAM 47-69 | CCACAUUCAUCAGAAUACA | GU | UGUAUUCUGAUGAAUGUG | CA |
| | (SEQ ID NO: 58) | | G (SEQ ID NO: 140) | |
| GPAM 1063- | CUAUGAUCGCAUUAUCGAA | GG | UUCGAUAAUGCGAUCAUAG | GA |
| 1085 | (SEQ ID NO: 59) | | (SEQ ID NO: 141) | |
| GPAM 604-626 | GAACAUUCAAAUUCACAAA | GG | UUUGUGAAUUUGAAUGUU | CA |
| | (SEQ ID NO: 60) | | C (SEQ ID NO: 142) | |
| GPAM 950-972 | CGUUCUAGGAGUGGAAAAA | CC | UUUUUCCACUCCUAGAACG | UG |
| | (SEQ ID NO: 61) | | (SEQ ID NO: 143) | |
| GPAM 624-646 | GUCAACUUGAGAUGGUUAA | AG | UUAACCAUCUCAAGUUGAC | CU |
| | (SEQ ID NO: 62) | | (SEQ ID NO: 144) | |
| GPAM 2440- | CCGACAAAAACUUCUAGAA | UA | UUCUAGAAGUUUUUGUCG | UU |
| 2462 | (SEQ ID NO: 63) | | G (SEQ ID NO: 145) | |
| J-009946-05 | CGAAGGAGGUUGAUUGCAA | UU | UUGCAAUCAACCUCCUUCG | UU |
| | (SEQ ID NO: 64) | | (SEQ ID NO: 146) | |
| J-009946-06 | GUAGAGACACGUCCAUUAA | UU | UUAAUGGACGUGUCUCUA | UU |
| | (SEQ ID NO: 65) | | C (SEQ ID NO: 147) | |
| J-009946-07 | GUUCAUAGAUCCCAUAUUG | UU | CAAUAUGGGAUCUAUGAAC | UU |
| | (SEQ ID NO: 66) | | (SEQ ID NO: 148) | |
| J-009946-08 | UAUCGAAGGUCACUACAAU | UU | AUUGUAGUGACCUUCGAUA | UU |
| | (SEQ ID NO: 67) | | (SEQ ID NO: 149) | |
| D-009946-01 | GGAAAGAUGUUCUCUAUAG | UU | CUAUAGAGAACAUCUUUCC | UU |
| | (SEQ ID NO: 68) | | (SEQ ID NO: 150) | |
| D-009946-02 | GAUAAUACCUGUUGGAAUC | UU | GAUUCCAACAGGUAUUAUC | UU |
| | (SEQ ID NO: 69) | | (SEQ ID NO: 151) | |
| D-009946-03 | GUUCAUAGAUCCCAUAUUG | UU | CAAUAUGGGAUCUAUGAAC | UU |
| | (SEQ ID NO: 70) | | (SEQ ID NO: 152) | |
| D-009946-04 | AGAAAGAAAUGUUGCAGUA | UU | UACUGCAACAUUUCUUUCU | UU |
| | (SEQ ID NO: 71) | | (SEQ ID NO: 153) | |
| s224483 | GAGGUGUUAUUAGAAUGUU | TT | AACAUUCUAAUAACACCUC | TT |
| | (SEQ ID NO: 72) | | (SEQ ID NO: 154) | |
| s224484 | GUAGGAAAGUUUAUCCAGU | TT | ACUGGAUAAACUUUCCUAC | TG |
| | (SEQ ID NO: 73) | | (SEQ ID NO: 155) | |
| s33574 | CAUCAGUCUUCGAACUCAA | TT | UUGAGUUCGAAGACUGAU | GG |
| | (SEQ ID NO: 74) | | G (SEQ ID NO: 156) | |
| s33575 | GGCAUAUAGUUGAAUUACU | TT | AGUAAUUCAACUAUAUGCC | CA |
| | (SEQ ID NO: 75) | | (SEQ ID NO: 157) | |
| s33576 | CCAUCAGUCUUCGAACUCA | TT | UGAGUUCGAAGACUGAUG | GA |
| | (SEQ ID NO: 76) | | G (SEQ ID NO: 158) | |
| 112114 | GCAGCAAUCAAAAGCCGUU | TT | AACGGCUUUUGAUUGCUG | TG |
| | (SEQ ID NO: 77) | | C (SEQ ID NO: 159) | |
| 112115 | GGUCACUACAAUGGUGAAC | TT | GUUCACCAUUGUAGUGAC | TT |
| | (SEQ ID NO: 78) | | C (SEQ ID NO: 160) | |
| 112116 | GCUAUAUGAAAACAGACAA | TT | UUGUCUGUUUUCAUAUAGC | TC |
| | (SEQ ID NO: 79) | | (SEQ ID NO: 161) | |
| 117624 | GGUGUUAUUAGAAUGUUAC | TT | GUAACAUUCUAAUAACACC | TC |
| | (SEQ ID NO: 80) | | (SEQ ID NO: 162) | |
| 43864 | GUGUUGGUCGAUGUAAGCA | TT | UGCUUACAUCGACCAACAC | TG |
| | (SEQ ID NO: 81) | | (SEQ ID NO: 163) | |
| 43960 | GCACACAAGUGAGGAAUGG | TT | CCAUUCCUCACUUGUGUG | TT |
| | (SEQ ID NO: 82) | | C (SEQ ID NO: 164) | |
| 44047 | GCCGUUAACAAAGUGAAAA | TT | UUUUCACUUUGUUAACGGC | TT |
| | (SEQ ID NO: 83) | | (SEQ ID NO: 165) | |

In some embodiments, double stranded siRNA molecules disclosed herein may have at least one sense sequence having at least about 80% homology (e.g., about 80%, about 85%, about 90%, about 95%, or about 99%) to any one of SEQ ID NOs: 2-83. In some embodiments, double stranded siRNA molecules disclosed herein may have at least one antisense sequence having at least about 80% homology (e.g., about 80%, about 85%, about 90%, about 95%, or about 99%) to any one of SEQ ID NOs: 84-165. In some embodiments, double stranded siRNA molecules disclosed herein may have at least one sense sequence having at least about 80% homology (e.g., about 80%, about 85%, about 90%, about 95%, or about 99%) to any one of SEQ ID NOs: 2-83 and at least one antisense sequence having at least about 80% homology (e.g., about 80%, about 85%, about 90%, about 95%, or about 99%) to any one of SEQ ID NOs: 84-165. In some embodiments, double stranded siRNA molecules disclosed herein may have at least one sense sequence of SEQ ID NOs: 2-83. In some embodiments, double stranded siRNA molecules disclosed herein may have at least one antisense sequence of SEQ ID NOs: 84-165. In some embodiments, double stranded siRNA molecules disclosed herein may have at least one sense sequence of SEQ ID NOs: 2-83 and at least one antisense sequence of SEQ ID NOs: 84-165. In some embodiments, double stranded siRNA molecules disclosed herein may have at least one sense sequence of SEQ ID NOs: 2-63. In some embodiments, double stranded siRNA molecules disclosed herein may have at least one antisense sequence of SEQ ID NOs: 84-145. In some embodiments, double stranded siRNA molecules disclosed herein may have at least one sense sequence of SEQ ID NOs: 2-63 and at least one antisense sequence of SEQ ID NOs: 84-145. In some aspects, double stranded siRNA molecules disclosed herein may have a sense sequence of SEQ ID NO: 8 and an antisense sequence of SEQ ID NO: 90. In some other aspects, double stranded siRNA molecules disclosed herein may have a sense sequence of SEQ ID NO: 9 and an antisense sequence of SEQ ID NO: 91. In yet some other aspects, double stranded siRNA molecules disclosed herein may have a sense sequence of SEQ ID NO: 13 and an antisense sequence of SEQ ID NO: 95. In still some other aspects, double stranded siRNA molecules disclosed herein may have a sense sequence of SEQ ID NO: 17 and an antisense sequence of SEQ ID NO: 99.

In some embodiments, the present disclosure also encompasses nucleic acid sequences encoding a double stranded siRNA molecule as disclosed herein. In some embodiments, a nucleic acid sequence encoding the double stranded siRNA as disclosed herein may be a double stranded DNA, single stranded DNA, a plasmid vector, a viral vector for example a retroviral vector, lentiviral vector, a pox viral vector, an adenoviral vector, or an adeno-associated viral vector. In some embodiments, the nucleic acid may encode at least one sense sequence having at least about 80% homology (e.g., about 80%, about 85%, about 90%, about 95%, or about 99%) to any one of SEQ ID NOs: 2-83. In some embodiments, the nucleic acids may encode at least one antisense sequence having at least about 80% homology (e.g., about 80%, about 85%, about 90%, about 95%, or about 99%) to any one of SEQ ID NOs: 84-165. In some embodiments, the nucleic acids may encode at least one sense sequence having at least about 80% homology (e.g., about 80%, about 85%, about 90%, about 95%, or about 99%) to any one of SEQ ID NOs: 2-83 and at least one antisense sequence having at least about 80% homology (e.g., about 80%, about 85%, about 90%, about 95%, or about 99%) to any one of SEQ ID NOs: 84-165. In some embodiments, the nucleic acids may encode at least one sense sequence of any one of SEQ ID NOs: 2-83. In some embodiments, the nucleic acids may encode at least one antisense sequence of any one of SEQ ID NOs: 84-165. In some embodiments, the nucleic acids may encode at least one sense sequence of SEQ ID NOs: 2-58 and at least one antisense sequence of SEQ ID NOs: 84-165. In some embodiments, the nucleic acid may encode a sense sequence of SEQ ID NO: 9 and an antisense sequence of SEQ ID NO: 91. In some aspects, the nucleic acid may encode a sense sequence of SEQ ID NO: 13 and an antisense sequence of SEQ ID NO: 95. In some aspects, nucleic acid molecule may encode a sense sequence of SEQ ID NO: 17 and an antisense sequence of SEQ ID NO: 99.

The present disclosure also provides for methods of making the siRNA molecules disclosed herein. Making of siRNAs may be through chemical synthesis or siRNA may be encoded by a plasmid and transcribed or may be vectored by a virus engineered to express the siRNA. A siRNA may be a single stranded molecule with complementary sequences that self-hybridize into duplexes with hairpin loops. siRNA can also be generated by cleavage of parent dsRNA through the use of an appropriate enzyme such as E. coli RNase Ill or Dicer. A parent dsRNA may be any double stranded RNA duplex from which a siRNA may be produced, such as a full or partial mRNA transcript. Use of cell lysates or in vitro processing may further involve the subsequent isolation of the short, nucleotide siRNAs (e.g., about 2-25 nucleotides in length) from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse and can be easily adapted to synthesis different siRNAs.

In certain embodiments, siRNA molecules disclosed herein may abolish gene expression of GPAM. Methods known in the art for the detection and quantification of RNA expression suitable for use herein can include, but are not limited to northern blotting and in situ hybridization, RNAse protection assays, polymerase chain reaction (PCR), reverse transcription polymerase chain reaction (RT-PCR), real-time quantitative reverse transcription PCR (RT-qPCR or qPCR), sequencing-based gene expression analysis (e.g., Serial Analysis of Gene Expression (SAGE)), gene expression analysis by massively parallel signature sequencing (MPSS), and the like.

The siRNA molecules of the present invention specifically downregulate gene expression of GPAM (Glycerol-3-Phosphate Acyltransferase, Mitochondrial) or a variant thereof. The term downregulating may be interchangeably expressed as reducing, inhibiting, preventing, blocking or silencing. Here, the phrase “downregulating gene expression” refers to any reduced level of gene expression comparing with an ordinary expression level. For example, the reduced level of gene expression can be from about 70% to 0% of the ordinary expression level. In other words, about 30% to 100% gene expression is downregulated, reduced, blocked, inhibited, prevented or silenced, comparing to the ordinary expression level. Specifically, the reduced level of gene expression is about 70%, 65%, 60%, 55%, 50%, 55%, 50%, 45%, 40%, 35%, 30%, 25%, 20%, 15%, 10%, 8%, 5%, 4%, 3%, 2%, 1% or 0% of the ordinary expression level of GPAM. The reduced level of gene expression can also be any percentage or range as recited above. On the other hand, the term “specific” or “specifically” used in combination with downregulating refers to downregulation of a target gene's expression with minimal or no binding or downregulation of other nucleic acids or their expressions.

In some embodiments, siRNA molecules disclosed herein may reduce gene expression of GPAM by at least about 50%. In some aspects, siRNA molecules disclosed herein may reduce gene expression of GPAM by about 50% to about 99%, about 55% to about 98%, or about 60% to about 95%. In some aspects, siRNA molecules disclosed herein may reduce gene expression of GPAM by about 50%, about 55%, about 60%, about 65%, about 70%, about 75%, about 80%, about 85%, about 90%, about 95%, or about 99%.

In certain embodiments, siRNA molecules disclosed herein may abolish protein expression of Gpam. Methods known in the art for the detection and quantification of protein expression suitable for use herein can include, but are not limited to ELISAs (enzyme-linked immunosorbent assays), immunoblot assays, flow cytometric assays, immunohistochemical assays, radioimmuno assays, Western blot assays, an immunofluorescent assays, chemiluminescent assays, mass spectrometry assays, matrix-assisted laser desorption/ionization time-of-flight (MALDI-TOF) mass mapping, liquid chromatography/quadrupole time-of-flight electrospray ionization tandem mass spectrometry (LC/Q-TOF-ESI-MS/MS), two-dimensional polyacrylamide gel electrophoresis (2D-PAGE), and the like.

In some embodiments, siRNA molecules disclosed herein may reduce protein expression of Gpam by at least about 50%. In some aspects, siRNA molecules disclosed herein may reduce protein of Gpam by about 50% to about 99%, about 55% to about 98%, or about 60% to about 95%. In some aspects, siRNA molecules disclosed herein may reduce protein expression of Gpam by about 50%, about 55%, about 60%, about 65%, about 70%, about 75%, about 80%, about 85%, about 90%, about 95%, or about 99%.

In some embodiments, siRNA molecule disclosed herein may have one or more chemical modifications. Non-limiting examples of chemical modifications can include terminal cap moieties, phosphate backbone modifications, and the like. Examples of classes of terminal cap moieties include, without limitation, inverted deoxy abasic residues, glyceryl modifications, 4′,5′-methylene nucleotides, 1-(β-D-erythrofuranosyl) nucleotides, 4′-thio nucleotides, carbocyclic nucleotides, 1,5-anhydrohexitol nucleotides, L-nucleotides, α-nucleotides, modified base nucleotides, threo pentofuranosyl nucleotides, acyclic 3′,4′-seco nucleotides, acyclic 3,4-dihydroxybutyl nucleotides, acyclic 3,5-dihydroxypentyl nucleotides, 3′-3′-inverted nucleotide moieties, 3′-3′-inverted abasic moieties, 3′-2′-inverted nucleotide moieties, 3′-2′-inverted abasic moieties, 5′-5′-inverted nucleotide moieties, 5′-5′-inverted abasic moieties, 3′-5′-inverted deoxy abasic moieties, 5′-amino-alkyl phosphate, 1,3-diamino-2-propyl phosphate, 3 aminopropyl phosphate, 6-aminohexyl phosphate, 1,2-aminododecyl phosphate, hydroxypropyl phosphate, 1,4-butanediol phosphate, 3′-phosphoramidate, 5′ phosphoramidate, hexylphosphate, aminohexyl phosphate, 3′-phosphate, 5′-amino, 3′-phosphorothioate, 5′-phosphorothioate, phosphorodithioate, and bridging or non-bridging methylphosphonate or 5′-mercapto moieties. Non-limiting examples of phosphate backbone modifications (i.e., resulting in modified internucleotide linkages) include phosphorothioate, phosphorodithioate, methylphosphonate, phosphotriester, morpholino, amidate, carbamate, carboxymethyl, acetamidate, polyamide, sulfonate, sulfonamide, sulfamate, formacetal, thioformacetal, and alkylsilyl substitutions. Such chemical modifications can occur at the 5′-end and/or 3′-end of the sense strand, antisense strand, or both strands of the siRNA.

Chemical modification of a siRNA molecule disclosed herein may comprise modification of at least one ribosugar moiety of its nucleotide sequence. The ribosugar moiety may be modified with 2 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, 2′-C-allyl, or any combination thereof. In some aspects, a siRNA molecule disclosed herein may have less than about 10% to about 70% (e.g., about 10%, about 20%, about 30%, about 40%, about 50%, about 60%, about 70%) of ribosugar moieties of the total nucleotide sequence modified. Chemical modification of a siRNA molecule disclosed herein may comprise attaching a conjugate to the siRNA molecule. The type of conjugate used and the extent of conjugation to the siRNA can be evaluated for improved pharmacokinetic profiles, bioavailability, and/or stability of the siRNA while retaining activity. As such, one skilled in the art can screen siRNA molecules having various conjugates attached thereto to identify siRNA conjugates having improved properties using any of a variety of well-known in vitro cell culture or in vivo animal models including the negative-controlled expression studies described above. The conjugate can be attached at the 5′- and/or the 3′-end of the sense and/or the antisense strand of the siRNA via a covalent attachment such as a nucleic acid or non-nucleic acid linker. The conjugate can be attached to the siRNA through a carbamate group or other linking group (see, e.g., U.S. Patent Publication Nos. 20050074771, 20050043219, and 20050158727, the content of each of which is incorporated by reference herein in its entirety). A conjugate may be added to siRNA for any of a number of purposes. For example, the conjugate may be a molecular entity that facilitates the delivery of siRNA into a cell or may be a molecule that comprises a drug or label. Examples of conjugate molecules suitable for attachment to siRNA of the present invention include, without limitation, steroids such as cholesterol, glycols such as polyethylene glycol (PEG), human serum albumin (HSA), fatty acids, carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, and combinations thereof. Other examples include the lipophilic moiety, vitamin, polymer, peptide, protein, nucleic acid, small molecule, oligosaccharide, carbohydrate cluster, intercalator, minor groove binder, cleaving agent, and cross-linking agent conjugate molecules described in e.g., U.S. Patent Publication Nos. 20050119470 and 20050107325, the content of each of which is incorporated by reference herein in its entirety. Other examples include the 2′-O-alkyl amine, 2′-O-alkoxyalkyl amine, polyamine, C5-cationic modified pyrimidine, cationic peptide, guanidinium group, amidininium group, cationic amino acid conjugate molecules, and the like. Additional examples of conjugate molecules include a hydrophobic group, a membrane active compound, a cell penetrating compound, a cell targeting signal, an interaction modifier, or a steric stabilizer as described in U.S. Patent Publication No. 20040167090, incorporated by reference herein in its entirety.

In certain embodiments, siRNA molecules disclosed herein may be conjugated to at least one targeting ligand. Targeting ligands contemplated herein include ligands suitable for targeting siRNA molecules to a liver, a liver tissue, and/or a liver cell. Non-limiting examples of targeting ligands suitable for use herein may include galactose, galactosamine, N-formyl-galactosamine, N-acetylgalactosamine, N-propionyl-galactosamine, N-n-butanoyl-galactosamine, N-iso-butanoylgalactos-amine, galactose cluster, and N-acetylgalactosamine trimer and may optionally have a pharmacokinetic modulator selected from the group consisting of: hydrophobic group having 16 or more carbon atoms, hydrophobic group having 16-20 carbon atoms, palmitoyl, hexadec-8-enoyl, oleyl, (9E,12E)-octadeca-9,12dienoyl, dioctanoyl, and C16-C20 acyl, and cholesterol. In some aspects, a liver targeting ligand suitable for use herein may be a N-Acetylgalactosamine (GalNAc) conjugate. In some embodiments, siRNA molecules disclosed herein may be conjugated to at least one GalNAc conjugate. In some embodiments, siRNA molecules disclosed herein may be conjugated to about 1 to about 10 GalNAc conjugates, about 2 to about 9 GalNAc conjugates, or about 3 to about 8 GalNAc conjugates. In some embodiments, siRNA molecules disclosed herein may be conjugated to about 1, about 2, about 3, about 4, about 5, about 6, about 7, about 8, about 9, or about 10 GalNAc conjugates.

Any of the siRNA molecules disclosed herein may target the open reading frame or the 5′ or 3′ UTRs of the GPAM gene or at least one variant thereof. In some embodiments, at least one variant of GPAM may comprise rs10787429 C>T, rs7096937 T>C, rs11446981 T>TA, rs7898213 T>C, rs2792759 C>T, rs2297991 T>C, rs1129555 A>G, rs2254537 T>A, or any combination thereof. In some aspects, siRNA molecules disclosed herein can specifically downregulates gene expression of GPAM or a variant thereof, such as rs10787429 C>T, rs7096937 T>C, rs11446981 T>TA, rs7898213 T>C, rs2792759 C>T, rs2297991 T>C, rs1129555 A>G, rs2254537 T>A, or any combination thereof. The GPAM gene and/or its variant may be associated with a liver disease, liver disfunction, liver injury, and/or a liver damage. The liver disease may comprise fatty liver disease (FLD), alcohol-related liver disease (ARLD), non-alcoholic fatty liver disease (NAFLD), non-alcoholic steatohepatitis (NASH), end stage liver disease (cirrhosis) from any etiology, liver cancer, or any combination thereof. Liver damage may include cirrhosis, chronic infection of hepatitis B virus (HBV), chronic infection of hepatitis C virus (HCV), non-alcoholic fatty liver disease (NAFLD), non-alcoholic steatohepatitis (NASH), primary biliary cirrhosis (PBC), hereditary hemochromatosis, type 2 diabetes, obesity, tobacco use, alcohol abuse, long-term anabolic steroid use, tyrosinemia, alpha1-antitrypsin deficiency, porphyria cutanea tarda, glycogen storage diseases, Wilson disease, or any combination thereof.

In certain embodiments, methods and compositions provided herein can include a vector containing any one of the siRNA molecules disclosed herein. In some embodiments, a vector for use herein can be a viral vector. As used herein, the term “viral vector” can refer to a nucleic acid vector construct that includes at least one element of viral origin and has the capacity to be packaged into a viral vector particle and encodes at least an exogenous polynucleotide. In certain embodiments, the vector and/or particle can be utilized for the purpose of transferring any nucleic acids into cells either in vitro or in vivo. Numerous viral vectors are known in the art. The term virion can refer to a single infective viral particle. “Viral vector”, “viral vector particle” and “viral particle” also refer to a complete virus particle with its DNA or RNA core and protein coat as it exists outside the cell. Non-limiting examples of viral vectors for use herein can include adenoviruses, adeno-associated viruses (AAV), herpesviruses, retroviruses, lentiviruses, integrase defective lentiviruses (IDLV), and the like. In some embodiments, a viral vector disclosed herein can be a lentiviral vector. Examples of lentiviruses include, but are not limited to, human lentiviruses such as HIV (in particular HIV-1 or HIV-2), simian immunodeficiency virus (SIV), equine infectious anemia virus (EIAV), feline immunodeficiency virus (FIV), Caprine Arthritis Encephalitis Virus (CAEV), visna and progressive pneumonia viruses of sheep, baboon pseudotype viruses, bovine immunodeficiency virus (BIV), and the like. In some embodiments, siRNA molecules and/or vectors described herein can be prepared by conventional recombinant technology known to one of skill in the art. In other embodiments, siRNA molecules and/or vectors described herein can be prepared by a gene editing methods known in the art (e.g., by CRISPR). In certain embodiments, methods provided herein can include generating a cell to express any of the siRNA molecules and/or vectors described herein. In some embodiments, vectors, viral particles, and the like as contemplated herein may be encapsulated into a liposome for delivery to a subject.

In some aspects, the current disclosure also encompasses use of gene editing systems for example CRISPR based systems for abolishing or downregulating gene expression of GPAM. As such, the current disclosure also encompasses compositions comprising a nucleic acid sequence related to a cluster regularly interspaced short palindromic repeats (CRISPR) system, for example a single guide RNA (sgRNA), a CRISPR-RNA (crRNA), a trans-activating crRNA (tracrRNA), and further plasmid DNA (pDNA) or a viral vector encoding nucleic acid sequence related to a cluster regularly interspaced short palindromic repeats (CRISPR) system, that specifically target GPAM or variants thereof. In some aspects, the gene editing system comprises at least an sgRNA targeting GPAM and an RNA guided endonuclease, for example Cas9. In some aspects, the sgRNA target sequence has at least about 80% homology (e.g., about 80%, about 85%, about 90%, about 95%, or about 99%) to any one SEQ ID NO: 177-276. In some aspects, the sgRNA target sequence is at least about 80% identical to any one of SEQ ID NO: 177-276 as provided in Table 2. In some aspects, the sgRNA targets a sequence at least about 80%, or about 85%, or about 90%, or about 95% or about 100% identical to SEQ ID NO: 177-276. In some aspects, the sgRNA targets 20 consecutive nucleotides in any one of SEQ ID NO: 277-376. None of the gRNA as provided in Table 2 are predicted to target the last exon or have 10 bp off target match.

TABLE 2

| | | Guide with | | | | | |
|---|---|---|---|---|---|---|---|
| | | flanking | | | | | |
| | | sequence | | cut | | | |
| | SEQ | (10 bp before | SEQ | position | On- | | |
| gRNA | ID | and after the | ID | in | target | | Off-target |
| Sequence | NO: | guide + PAM) | NO: | chromosome | efficiency | Exon | score |
| AGGGAAA | 177 | CGCTTGCTCC | 277 | 1.14E+08 | 0.722105 | 15 | 0 |
| GTAGAGC | | AGGGAAAGTA | | | | | |
| AGACAC | | GAGCAGACAC | | | | | |
| | | CGGCGGTTTC | | | | | |
| | | TGA | | | | | |
| AAAGAAG | 178 | CTTTCATCAC | 278 | 1.14E+08 | 0.714797 | 18 | 0 |
| TCTTCGA | | AAAGAAGTCT | | | | | |
| CCAATG | | TCGACCAATG | | | | | |
| | | TGGTGGAGA | | | | | |
| | | GATC | | | | | |
| AGCCTGT | 179 | GAAGAATGAG | 279 | 1.14E+08 | 0.711 | 14 | 0 |
| GGAGTGT | | AGCCTGTGGA | | | | | |
| AGCAAG | | GTGTAGCAAG | | | | | |
| | | AGGAGGTGTT | | | | | |
| | | ATT | | | | | |
| CAGATGA | 180 | AGAAATGCAA | 280 | 1.14E+08 | 0.708861 | 16 | 0 |
| ATCCCTA | | CAGATGAATC | | | | | |
| CGAAGG | | CCTACGAAGG | | | | | |
| | | AGGAGGTTGA | | | | | |
| | | TTG | | | | | |
| TGAACTG | 181 | ATGGGATCTA | 281 | 1.14E+08 | 0.698231 | 11 | 0 |
| GTAGAAA | | TGAACTGGTA | | | | | |
| CAGAAG | | GAAACAGAAG | | | | | |
| | | CGGCGGCAA | | | | | |
| | | ATTC | | | | | |
| CAACAGA | 182 | TCCAGAAATG | 282 | 1.14E+08 | 0.697998 | 16 | 0 |
| TGAATCC | | CAACAGATGA | | | | | |
| CTACGA | | ATCCCTACGA | | | | | |
| | | AGGAGGAGG | | | | | |
| | | TTGA | | | | | |
| CTAAGAA | 183 | TAGGGCAAAC | 283 | 1.14E+08 | 0.680163 | 14 | 0 |
| GAATGAG | | CTAAGAAGAA | | | | | |
| AGCCTG | | TGAGAGCCTG | | | | | |
| | | TGGTGGAGTG | | | | | |
| | | TAG | | | | | |
| ACCTTGA | 184 | ACCTTGATCC | 284 | 1.14E+08 | 0.674072 | 12 | 0 |
| TCCATAA | | ATAAGCTTGG | | | | | |
| GCTTGG | | GGGGGGCTT | | | | | |
| | | CTTC | | | | | |
| TCTGAAC | 185 | TTTATGCAGT | 285 | 1.14E+08 | 0.666287 | 19 | 0 |
| AAGAGGG | | TCTGAACAAG | | | | | |
| GACTGG | | AGGGGACTG | | | | | |
| | | GGGGGGGGT | | | | | |
| | | CCCAC | | | | | |
| CAAGTCT | 186 | GACATTTTAC | 286 | 1.14E+08 | 0.663633 | 20 | 0 |
| GCCATGA | | CAAGTCTGCC | | | | | |
| AACAGT | | ATGAAACAGT | | | | | |
| | | AGGAGGAAA | | | | | |
| | | GTTT | | | | | |
| ACTTCAT | 187 | GCAATGGGGT | 287 | 1.14E+08 | 0.663112 | 18 | 0 |
| GTCTTTA | | ACTTCATGTC | | | | | |
| TCATGG | | TTTATCATGG | | | | | |
| | | AGGAGGCCAT | | | | | |
| | | CAT | | | | | |
| GAAGGCA | 188 | GATCTTCCTG | 288 | 1.14E+08 | 0.66057 | 13 | 0 |
| CACGTTC | | GAAGGCACAC | | | | | |
| TAGGAG | | GTTCTAGGAG | | | | | |
| | | TGGTGGAAAA | | | | | |
| | | ACC | | | | | |
| AGGCTCG | 189 | CATACGACGA | 289 | 1.14E+08 | 0.657391 | 12 | 0 |
| ATGAAAC | | AGGCTCGATG | | | | | |
| ACCAGA | | AAACACCAGA | | | | | |
| | | TGGTGGACG | | | | | |
| | | GAAA | | | | | |
| TGATGGG | 190 | GTTCGAAGAC | 290 | 1.14E+08 | 0.648279 | 18 | 0 |
| ACAGTTG | | TGATGGGACA | | | | | |
| TGCTGG | | GTTGTGCTGG | | | | | |
| | | GGGGGGTGA | | | | | |
| | | TAAA | | | | | |
| GATCATT | 191 | GATCATTGCC | 291 | 1.14E+08 | 0.648127 | 9 | 0 |
| GCCGGT | | GGTGAGACA | | | | | |
| GAGACAG | | GTGGTGGCAA | | | | | |
| | | CCAT | | | | | |
| AAACTAT | 192 | TGTTACGAAA | 292 | 1.14E+08 | 0.644678 | 14 | 0 |
| GGTTGTG | | AAACTATGGT | | | | | |
| TCCGAG | | TGTGTCCGAG | | | | | |
| | | TGGTGGATTT | | | | | |
| | | TGC | | | | | |
| GGGGCTT | 193 | CATAAGCTTG | 293 | 1.14E+08 | 0.644507 | 12 | 0 |
| CTTCATA | | GGGGCTTCTT | | | | | |
| CGACGA | | CATACGACGA | | | | | |
| | | AGGAGGCTC | | | | | |
| | | GATG | | | | | |
| CAGGCAA | 194 | TGTAGAGGAG | 294 | 1.14E+08 | 0.640983 | 17 | 0 |
| GCCACAA | | CAGGCAAGC | | | | | |
| TGTGTG | | CACAATGTGT | | | | | |
| | | GTGGTGGACA | | | | | |
| | | TAAT | | | | | |
| TCAGCCA | 195 | CCTAACCTGA | 295 | 1.14E+08 | 0.640431 | 19 | 0.154589 |
| GGAGCA | | TCAGCCAGGA | | | | | |
| GCTGGTG | | GCAGCTGGT | | | | | |
| | | GCGGCGGAA | | | | | |
| | | GGCGG | | | | | |
| CCCACCT | 196 | CCACTAGCAC | 296 | 1.14E+08 | 0.633645 | 19 | 0 |
| AACCTGA | | CCCACCTAAC | | | | | |
| TCAGCC | | CTGATCAGCC | | | | | |
| | | AGGAGGAGC | | | | | |
| | | AGCT | | | | | |
| ATGGCTG | 197 | TTTAAGGAAA | 297 | 1.14E+08 | 0.631299 | 14 | 0 |
| TGCAAAA | | ATGGCTGTGC | | | | | |
| TCCACT | | AAAATCCACT | | | | | |
| | | CGGCGGACA | | | | | |
| | | CAAC | | | | | |
| TTTATGC | 198 | GCTTGCAGCC | 298 | 1.14E+08 | 0.628756 | 19 | 0 |
| AGTTCTG | | TTTATGCAGT | | | | | |
| AACAAG | | TCTGAACAAG | | | | | |
| | | AGGAGGGGA | | | | | |
| | | CTGG | | | | | |
| CATTATG | 199 | AGTCCTGTGC | 299 | 1.14E+08 | 0.62442 | 17 | 0 |
| TCCACAC | | CATTATGTCC | | | | | |
| ACATTG | | ACACACATTG | | | | | |
| | | TGGTGGCTTG | | | | | |
| | | CCT | | | | | |
| CAAGATG | 200 | CAGGTATTAT | 300 | 1.14E+08 | 0.619742 | 13 | 0.026087 |
| TCTGGGA | | CAAGATGTCT | | | | | |
| TGACAT | | GGGATGACAT | | | | | |
| | | TGGTGGTAGA | | | | | |
| | | CAG | | | | | |
| GGAGAGA | 201 | TGCCTTCATT | 301 | 1.14E+08 | 0.615343 | 19 | 0.100294 |
| AGGTAGC | | GGAGAGAAG | | | | | |
| ACAGGC | | GTAGCACAGG | | | | | |
| | | CTGGTGGCC | | | | | |
| | | GCCTT | | | | | |
| TCGATGA | 202 | CGACGAAGG | 302 | 1.14E+08 | 0.613804 | 12 | 0 |
| AACACCA | | CTCGATGAAA | | | | | |
| GATGGA | | CACCAGATGG | | | | | |
| | | ACGGCGGAA | | | | | |
| | | AGATG | | | | | |
| CCAGTAT | 203 | GAAAGTTTAT | 303 | 1.14E+08 | 0.613053 | 20 | 0 |
| GGCATTC | | CCAGTATGGC | | | | | |
| TTACAG | | ATTCTTACAG | | | | | |
| | | TGGTGGCAGA | | | | | |
| | | GGT | | | | | |
| CACCTCT | 204 | ATTCTAATAA | 304 | 1.14E+08 | 0.603193 | 14 | 0 |
| TGCTACA | | CACCTCTTGC | | | | | |
| CTCCAC | | TACACTCCAC | | | | | |
| | | AGGAGGCTCT | | | | | |
| | | CAT | | | | | |
| TCACAAT | 205 | GGAAATTGTG | 305 | 1.14E+08 | 0.599371 | 18 | 0 |
| CACCCAC | | TCACAATCAC | | | | | |
| ACTAGC | | CCACACTAGC | | | | | |
| | | AGGAGGAAC | | | | | |
| | | GATG | | | | | |
| CATTGGA | 206 | ATGGTGCCTT | 306 | 1.14E+08 | 0.597178 | 19 | 0.19388 |
| GAGAAGG | | CATTGGAGAG | | | | | |
| TAGCAC | | AAGGTAGCAC | | | | | |
| | | AGGAGGCTG | | | | | |
| | | GCCG | | | | | |
| GCCTGCT | 207 | ATTGTGGCTT | 307 | 1.14E+08 | 0.596688 | 17 | 0 |
| CCTCTAC | | GCCTGCTCCT | | | | | |
| AGACAC | | CTACAGACAC | | | | | |
| | | AGGAGGCAG | | | | | |
| | | GTAT | | | | | |
| GTGTGTG | 208 | AAGCCACAAT | 308 | 1.14E+08 | 0.594049 | 17 | 0 |
| GACATAA | | GTGTGTGGAC | | | | | |
| TGGCAC | | ATAATGGCAC | | | | | |
| | | AGGAGGACTT | | | | | |
| | | GCT | | | | | |
| AGACTTC | 209 | CATTGGTCGA | 309 | 1.14E+08 | 0.592433 | 18 | 0 |
| TTTGTGA | | AGACTTCTTT | | | | | |
| TGAAAG | | GTGATGAAAG | | | | | |
| | | AGGAGGAAGT | | | | | |
| | | CCT | | | | | |
| ATTATCG | 210 | CTATGATCGC | 310 | 1.14E+08 | 0.59151 | 13 | 0 |
| AAGGTCA | | ATTATCGAAG | | | | | |
| CTACAA | | GTCACTACAA | | | | | |
| | | TGGTGGTGAA | | | | | |
| | | CAA | | | | | |
| AATGGTT | 211 | TTCTTCAAGA | 311 | 1.14E+08 | 0.586042 | 9 | 0 |
| GCCACTG | | AATGGTTGCC | | | | | |
| TCTCAC | | ACTGTCTCAC | | | | | |
| | | CGGCGGCAA | | | | | |
| | | TGAT | | | | | |
| TCTTGTT | 212 | CCCAGTCCCC | 312 | 1.14E+08 | 0.58577 | 19 | 0 |
| CAGAACT | | TCTTGTTCAG | | | | | |
| GCATAA | | AACTGCATAA | | | | | |
| | | AGGAGGCTG | | | | | |
| | | CAAG | | | | | |
| CCAGGTC | 213 | CCTGAGAACC | 313 | 1.14E+08 | 0.584857 | 18 | 0 |
| AAAATCA | | CCAGGTCAAA | | | | | |
| CGAGCC | | ATCACGAGCC | | | | | |
| | | AGGAGGACTT | | | | | |
| | | CCT | | | | | |
| GGAGCA | 214 | TGATCAGCCA | 314 | 1.14E+08 | 0.580939 | 19 | 1.685601 |
| GCTGGTG | | GGAGCAGCT | | | | | |
| CGGAAG | | GGTGCGGAA | | | | | |
| G | | GGCGGCGGC | | | | | |
| | | CAGCCT | | | | | |
| GGGAATT | 215 | GGGAATTGAT | 315 | 1.14E+08 | 0.57917 | 18 | 0 |
| GATCTCT | | CTCTCCACAT | | | | | |
| CCACAT | | TGGTGGTCGA | | | | | |
| | | AGA (right | | | | | |
| | | flank only) | | | | | |
| TTCTTGG | 216 | ACAGCAGCAA | 316 | 1.14E+08 | 0.575157 | 13 | 0.04 |
| AGATCTT | | TTCTTGGAGA | | | | | |
| CCTGGA | | TCTTCCTGGA | | | | | |
| | | AGGAGGCAC | | | | | |
| | | ACGT | | | | | |
| AAAGCAC | 217 | CCATAACATC | 317 | 1.14E+08 | 0.569069 | 11 | 0 |
| CATACAT | | AAAGCACCAT | | | | | |
| TGCTTC | | ACATTGCTTC | | | | | |
| | | AGGAGGCAAT | | | | | |
| | | AAT | | | | | |
| TCCTATG | 218 | TGTTGGAATC | 318 | 1.14E+08 | 0.565826 | 13 | 0 |
| ATCGCAT | | TCCTATGATC | | | | | |
| TATCGA | | GCATTATCGA | | | | | |
| | | AGGAGGTCAC | | | | | |
| | | TAC | | | | | |
| TCAATAT | 219 | CAGCAGATAG | 319 | 1.14E+08 | 0.564178 | 11 | 0 |
| GGGATCT | | TCAATATGGG | | | | | |
| ATGAAC | | ATCTATGAAC | | | | | |
| | | TGGTGGTAGA | | | | | |
| | | AAC | | | | | |
| AAAACCT | 220 | TAGGAGTGGA | 320 | 1.14E+08 | 0.55936 | 13 | 0 |
| CTTGTGC | | AAAACCTCTT | | | | | |
| TCGGGC | | GTGCTCGGG | | | | | |
| | | CAGGAGGACT | | | | | |
| | | TTTG | | | | | |
| TGCTACC | 221 | GGCCAGCCT | 321 | 1.14E+08 | 0.555599 | 19 | 0 |
| TTCTCTC | | GTGCTACCTT | | | | | |
| CAATGA | | CTCTCCAATG | | | | | |
| | | AAGGAGGCA | | | | | |
| | | CCATC | | | | | |
| TTATTGC | 222 | GATGTTGAGA | 322 | 1.14E+08 | 0.555348 | 11 | 0 |
| CTGAAGC | | TTATTGCCTG | | | | | |
| AATGTA | | AAGCAATGTA | | | | | |
| | | TGGTGGTGCT | | | | | |
| | | TTG | | | | | |
| TGGTGCC | 223 | ACCAGTGAGA | 323 | 1.14E+08 | 0.554125 | 19 | 0 |
| TTCATTG | | TGGTGCCTTC | | | | | |
| GAGAGA | | ATTGGAGAGA | | | | | |
| | | AGGAGGTAG | | | | | |
| | | CACA | | | | | |
| GAACTCA | 224 | ATCAGTCTTC | 324 | 1.14E+08 | 0.548374 | 18 | 0.436508 |
| ACTTCTA | | GAACTCAACT | | | | | |
| CAGCAA | | TCTACAGCAA | | | | | |
| | | TGGTGGGGTA | | | | | |
| | | CTT | | | | | |
| AAGTCCT | 225 | CAACTGACAA | 325 | 1.14E+08 | 0.548332 | 13 | 0 |
| GCCCGA | | AAGTCCTGCC | | | | | |
| GCACAAG | | CGAGCACAAG | | | | | |
| | | AGGAGGTTTT | | | | | |
| | | TCC | | | | | |
| ACCTTCG | 226 | CATTGTAGTG | 326 | 1.14E+08 | 0.540868 | 13 | 0 |
| ATAATGC | | ACCTTCGATA | | | | | |
| GATCAT | | ATGCGATCAT | | | | | |
| | | AGGAGGAGAT | | | | | |
| | | TCC | | | | | |
| TTGCAAT | 227 | TCAGCCAGAT | 327 | 1.14E+08 | 0.540004 | 16 | 0 |
| CAACCTC | | TTGCAATCAA | | | | | |
| CTTCGT | | CCTCCTTCGT | | | | | |
| | | AGGAGGGATT | | | | | |
| | | CAT | | | | | |
| GACATCT | 228 | TGTCATCCCA | 328 | 1.14E+08 | 0.539518 | 13 | 0 |
| TGATAAT | | GACATCTTGA | | | | | |
| ACCTGT | | TAATACCTGT | | | | | |
| | | TGGTGGAATC | | | | | |
| | | TCC | | | | | |
| GGTGTCT | 229 | GTCAGAAACC | 329 | 1.14E+08 | 0.533035 | 15 | 0.092105 |
| GCTCTAC | | GGTGTCTGCT | | | | | |
| TTTCCC | | CTACTTTCCC | | | | | |
| | | TGGTGGAGCA | | | | | |
| | | AGC | | | | | |
| TGTGATG | 230 | AAGACTTCTT | 330 | 1.14E+08 | 0.532868 | 18 | 0.546584 |
| AAAGAGG | | TGTGATGAAA | | | | | |
| AAGTCC | | GAGGAAGTCC | | | | | |
| | | TGGTGGCTCG | | | | | |
| | | TGA | | | | | |
| TCACAAA | 231 | ACATTCAAAT | 331 | 1.14E+08 | 0.524461 | 10 | 0 |
| GGTCAAC | | TCACAAAGGT | | | | | |
| TTGAGA | | CAACTTGAGA | | | | | |
| | | TGGTGGTTAA | | | | | |
| | | AGC | | | | | |
| CTGAATT | 232 | ACTACATCTT | 332 | 1.14E+08 | 0.523627 | 18 | 0 |
| TCCTGAG | | CTGAATTTCC | | | | | |
| AACCCC | | TGAGAACCCC | | | | | |
| | | AGGAGGTCAA | | | | | |
| | | AAT | | | | | |
| AAGTTGA | 233 | ATTGCTGTAG | 333 | 1.14E+08 | 0.522231 | 18 | 0 |
| GTTCGAA | | AAGTTGAGTT | | | | | |
| GACTGA | | CGAAGACTGA | | | | | |
| | | TGGTGGGACA | | | | | |
| | | GTT | | | | | |
| ACTCATC | 234 | GTGATAAAAA | 334 | 1.14E+08 | 0.521959 | 18 | 0 |
| GTTCCTG | | ACTCATCGTT | | | | | |
| CTAGTG | | CCTGCTAGTG | | | | | |
| | | TGGTGGGTGA | | | | | |
| | | TTG | | | | | |
| CGATCAT | 235 | TTCGATAATG | 335 | 1.14E+08 | 0.516878 | 13 | 0 |
| AGGAGAT | | CGATCATAGG | | | | | |
| TCCAAC | | AGATTCCAAC | | | | | |
| | | AGGAGGTATT | | | | | |
| | | ATC | | | | | |
| ACAATTT | 236 | TGATTGTGAC | 336 | 1.14E+08 | 0.514182 | 18 | 0.171123 |
| CCCAGCA | | ACAATTTCCC | | | | | |
| GCTGTA | | AGCAGCTGTA | | | | | |
| | | TGGTGGCATG | | | | | |
| | | CAT | | | | | |
| CCCAGTG | 237 | CCCAGTGATG | 337 | 1.14E+08 | 0.505139 | 16 | 0.631234 |
| ATGCTGC | | CTGCTGATGA | | | | | |
| TGATGA | | AGGAGGTAGA | | | | | |
| | | GAC (right | | | | | |
| | | flank only) | | | | | |
| CTACTGT | 238 | ATAAACTTTC | 338 | 1.14E+08 | 0.502412 | 20 | 0 |
| TTCATGG | | CTACTGTTTC | | | | | |
| CAGACT | | ATGGCAGACT | | | | | |
| | | TGGTGGTAAA | | | | | |
| | | ATG | | | | | |
| TGGAACA | 239 | CAGCTTCTTT | 339 | 1.14E+08 | 0.49856 | 10 | 0.096886 |
| TTCAAAT | | TGGAACATTC | | | | | |
| TCACAA | | AAATTCACAA | | | | | |
| | | AGGAGGTCAA | | | | | |
| | | CTT | | | | | |
| GGCAGAC | 240 | ACTGTTTCAT | 340 | 1.14E+08 | 0.497777 | 20 | 0 |
| TTGGTAA | | GGCAGACTTG | | | | | |
| AATGTC | | GTAAAATGTC | | | | | |
| | | TGGTGGCAAG | | | | | |
| | | GCT | | | | | |
| CAGGTTA | 241 | CCTGGCTGAT | 341 | 1.14E+08 | 0.496711 | 19 | 0 |
| GGTGGG | | CAGGTTAGGT | | | | | |
| GTGCTAG | | GGGGTGCTA | | | | | |
| | | GTGGTGGGA | | | | | |
| | | CCCCC | | | | | |
| TCCTGGC | 242 | CACCAGCTGC | 342 | 1.14E+08 | 0.490271 | 19 | 0 |
| TGATCAG | | TCCTGGCTGA | | | | | |
| GTTAGG | | TCAGGTTAGG | | | | | |
| | | TGGTGGGGT | | | | | |
| | | GCTA | | | | | |
| CACAATG | 243 | GCAGGCAAG | 343 | 1.14E+08 | 0.489022 | 17 | 0 |
| TGTGTGG | | CCACAATGTG | | | | | |
| ACATAA | | TGTGGACATA | | | | | |
| | | ATGGTGGCAC | | | | | |
| | | AGGA | | | | | |
| GCCTGTG | 244 | GCCTGTGTCT | 344 | 1.14E+08 | 0.488208 | 17 | 0 |
| TCTGTAG | | GTAGAGGAG | | | | | |
| AGGAGC | | CAGGAGGCA | | | | | |
| | | AGCCA (right | | | | | |
| | | flank only) | | | | | |
| CCAGGAG | 245 | ACCTGATCAG | 345 | 1.14E+08 | 0.487085 | 19 | 0.994892 |
| CAGCTGG | | CCAGGAGCA | | | | | |
| TGCGGA | | GCTGGTGCG | | | | | |
| | | GAAGGAGGC | | | | | |
| | | GGCCAG | | | | | |
| CCAGCTG | 246 | GCCTTCCGCA | 346 | 1.14E+08 | 0.483533 | 19 | 0.634785 |
| CTCCTGG | | CCAGCTGCTC | | | | | |
| CTGATC | | CTGGCTGATC | | | | | |
| | | AGGAGGTTAG | | | | | |
| | | GTG | | | | | |
| CCACTGT | 247 | CTTACCTCTG | 347 | 1.14E+08 | 0.483431 | 20 | 0 |
| AAGAATG | | CCACTGTAAG | | | | | |
| CCATAC | | AATGCCATAC | | | | | |
| | | TGGTGGATAA | | | | | |
| | | ACT | | | | | |
| AGAACAT | 248 | AGCTCTATAG | 348 | 1.14E+08 | 0.475583 | 12 | 0 |
| CTTTCCG | | AGAACATCTT | | | | | |
| TCCATC | | TCCGTCCATC | | | | | |
| | | TGGTGGTGTT | | | | | |
| | | TCA | | | | | |
| GTAGGAA | 249 | CCATGAAACA | 349 | 1.14E+08 | 0.475139 | 20 | 0 |
| AGTTTAT | | GTAGGAAAGT | | | | | |
| CCAGTA | | TTATCCAGTA | | | | | |
| | | TGGTGGCATT | | | | | |
| | | CTT | | | | | |
| AGTTCTG | 250 | GCCTTTATGC | 350 | 1.14E+08 | 0.475057 | 19 | 0 |
| AACAAGA | | AGTTCTGAAC | | | | | |
| GGGGAC | | AAGAGGGGA | | | | | |
| | | CTGGTGGGG | | | | | |
| | | GGTCC | | | | | |
| CCTGATC | 251 | CCCCACCTAA | 351 | 1.14E+08 | 0.473875 | 19 | 0.980201 |
| AGCCAGG | | CCTGATCAGC | | | | | |
| AGCAGC | | CAGGAGCAG | | | | | |
| | | CTGGTGGTGC | | | | | |
| | | GGAA | | | | | |
| AGAAAGC | 252 | AGGAATATTT | 352 | 1.14E+08 | 0.473237 | 15 | 0 |
| CAAAGTC | | AGAAAGCCAA | | | | | |
| AGAAAC | | AGTCAGAAAC | | | | | |
| | | CGGCGGTGT | | | | | |
| | | CTGC | | | | | |
| ACCTTCA | 253 | ACGTGTCTCT | 353 | 1.14E+08 | 0.468881 | 16 | 0 |
| TCAGCAG | | ACCTTCATCA | | | | | |
| CATCAC | | GCAGCATCAC | | | | | |
| | | TGGTGGGTCT | | | | | |
| | | AAA | | | | | |
| CAGACAC | 254 | GAAAGTAGAG | 354 | 1.14E+08 | 0.465083 | 15 | 0 |
| CGGTTTC | | CAGACACCG | | | | | |
| TGACTT | | GTTTCTGACT | | | | | |
| | | TTGGTGGCTT | | | | | |
| | | TCTA | | | | | |
| CCTTCCG | 255 | AGGCTGGCC | 355 | 1.14E+08 | 0.465069 | 19 | 1.747269 |
| CACCAGC | | GCCTTCCGCA | | | | | |
| TGCTCC | | CCAGCTGCTC | | | | | |
| | | CTGGTGGCTG | | | | | |
| | | ATCA | | | | | |
| TGCTCCT | 256 | CCGCACCAG | 356 | 1.14E+08 | 0.462775 | 19 | 0.093426 |
| GGCTGAT | | CTGCTCCTGG | | | | | |
| CAGGTT | | CTGATCAGGT | | | | | |
| | | TAGGAGGTG | | | | | |
| | | GGGTG | | | | | |
| TCCTGGA | 257 | TTGGAGATCT | 357 | 1.14E+08 | 0.461828 | 13 | 0 |
| AGGCACA | | TCCTGGAAGG | | | | | |
| CGTTCT | | CACACGTTCT | | | | | |
| | | AGGAGGAGT | | | | | |
| | | GGAA | | | | | |
| GACTGAT | 258 | TGAGTTCGAA | 358 | 1.14E+08 | 0.456769 | 18 | 0 |
| GGGACA | | GACTGATGGG | | | | | |
| GTTGTGC | | ACAGTTGTGC | | | | | |
| | | TGGTGGGGG | | | | | |
| | | TGAT | | | | | |
| CTGGTAA | 259 | GGAAGTATAG | 359 | 1.14E+08 | 0.445123 | 15 | 0 |
| CAACGCT | | CTGGTAACAA | | | | | |
| TGCTCC | | CGCTTGCTCC | | | | | |
| | | AGGAGGGAA | | | | | |
| | | AGTA | | | | | |
| ACAGGTA | 260 | GGAGATTCCA | 360 | 1.14E+08 | 0.438797 | 13 | 0 |
| TTATCAA | | ACAGGTATTA | | | | | |
| GATGTC | | TCAAGATGTC | | | | | |
| | | TGGTGGGATG | | | | | |
| | | ACA | | | | | |
| GGTACTT | 261 | ACAGCAATGG | 361 | 1.14E+08 | 0.431181 | 18 | 0 |
| CATGTCT | | GGTACTTCAT | | | | | |
| TTATCA | | GTCTTTATCA | | | | | |
| | | TGGTGGAGG | | | | | |
| | | CCAT | | | | | |
| AATGCAT | 262 | AAGATGTAGT | 362 | 1.14E+08 | 0.429229 | 18 | 0 |
| GCCATAC | | AATGCATGCC | | | | | |
| AGCTGC | | ATACAGCTGC | | | | | |
| | | TGGTGGGAAA | | | | | |
| | | TTG | | | | | |
| CAGTGAG | 263 | CAGTGAGATG | 363 | 1.14E+08 | 0.417979 | 19 | 0.111264 |
| ATGGTGC | | GTGCCTTCAT | | | | | |
| CTTCAT | | TGGTGGAGA | | | | | |
| | | GAAG | | | | | |
| AAGGAGG | 264 | AATCCCTACG | 364 | 1.14E+08 | 0.41192 | 16 | 0 |
| TTGATTG | | AAGGAGGTTG | | | | | |
| CAAATC | | ATTGCAAATC | | | | | |
| | | TGGTGGCTGA | | | | | |
| | | GCA | | | | | |
| GCAATTC | 265 | TTCGACAGCA | 365 | 1.14E+08 | 0.411615 | 13 | 0 |
| TTGGAGA | | GCAATTCTTG | | | | | |
| TCTTCC | | GAGATCTTCC | | | | | |
| | | TGGTGGAAG | | | | | |
| | | GCAC | | | | | |
| TGCTGCT | 266 | GGCTTTTGAT | 366 | 1.14E+08 | 0.400186 | 9 | 0 |
| GGGCAG | | TGCTGCTGGG | | | | | |
| AACCATC | | CAGAACCATC | | | | | |
| | | AGGAGGGTTT | | | | | |
| | | AAT | | | | | |
| TGAGCAG | 267 | AGAATGAAAG | 367 | 1.14E+08 | 0.393929 | 11 | 0 |
| CAGATAG | | TGAGCAGCAG | | | | | |
| TCAATA | | ATAGTCAATA | | | | | |
| | | TGGTGGGATC | | | | | |
| | | TAT | | | | | |
| ACTTCGA | 268 | TAGTTGAATT | 368 | 1.14E+08 | 0.390724 | 13 | 0 |
| CAGCAGC | | ACTTCGACAG | | | | | |
| AATTCT | | CAGCAATTCT | | | | | |
| | | TGGTGGAGAT | | | | | |
| | | CTT | | | | | |
| TAAACTT | 269 | CCATACTGGA | 369 | 1.14E+08 | 0.34851 | 20 | 0 |
| TCCTACT | | TAAACTTTCC | | | | | |
| GTTTCA | | TACTGTTTCA | | | | | |
| | | TGGTGGCAGA | | | | | |
| | | CTT | | | | | |
| TCCTAGA | 270 | GTTTTTCCAC | 370 | 1.14E+08 | 0.318432 | 13 | 0 |
| ACGTGTG | | TCCTAGAACG | | | | | |
| CCTTCC | | TGTGCCTTCC | | | | | |
| | | AGGAGGAAG | | | | | |
| | | ATCT | | | | | |
| CACAGGC | 271 | TGCTACACTC | 371 | 1.14E+08 | 0.317196 | 14 | 0.295455 |
| TCTCATT | | CACAGGCTCT | | | | | |
| CTTCTT | | CATTCTTCTTA | | | | | |
| | | GGAGGTTTGC | | | | | |
| | | CC | | | | | |
| CTCTATA | 272 | GAAAGATGTT | 372 | 1.14E+08 | 0.313792 | 12 | 0 |
| GAGCTTT | | CTCTATAGAG | | | | | |
| GCTCCA | | CTTTGCTCCA | | | | | |
| | | TGGTGGGGTA | | | | | |
| | | TG | | | | | |
| TGTATGG | 273 | CCTGAAGCAA | 373 | 1.14E+08 | 0.303238 | 11 | 0 |
| TGCTTTG | | TGTATGGTGC | | | | | |
| ATGTTA | | TTTGATGTTAT | | | | | |
| | | GGTGGCAGA | | | | | |
| | | AGA | | | | | |
| TGCATTT | 274 | ATTCATCTGT | 374 | 1.14E+08 | 0.238517 | 16 | 0 |
| CTGGACT | | TGCATTTCTG | | | | | |
| CATTAA | | GACTCATTAA | | | | | |
| | | TGGTGGACGT | | | | | |
| | | GTC | | | | | |
| AACTGTT | 275 | GTGCTGCTAA | 375 | 1.14E+08 | 0.233186 | 10 | 0 |
| CAACAGC | | AACTGTTCAA | | | | | |
| TTCTTT | | CAGCTTCTTT | | | | | |
| | | TGGTGGAACA | | | | | |
| | | TTC | | | | | |
| GGATTCA | 276 | TCCTTCGTAG | 376 | 1.14E+08 | 0.088445 | 16 | 0 |
| TCTGTTG | | GGATTCATCT | | | | | |
| CATTTC | | GTTGCATTTC | | | | | |
| | | TGGTGGACTC | | | | | |
| | | ATT | | | | | |

### (b) Pharmaceutical Compositions

The siRNA molecules targeting GPAM disclosed herein for use according to the methods herein described may be provided per se and/or as part of a pharmaceutical composition, where modulators and/or inhibitors can be mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism. Herein the term “active ingredient” refers to any of the siRNA molecules disclosed herein. The term “active ingredient” as used herein can also include any vector, medium, microorganism, or cell culture wherein the siRNA molecule is synthesized, expressed and/or contained, such as a genetically modified cell, viral vector, plasmid, bacteria, yeast, fungus, and the culture or medium thereof.

### (i) Pharmaceutically Acceptable Carriers and Excipients

Hereinafter, the phrases “physiologically acceptable carrier” and “pharmaceutically acceptable carrier” are interchangeably used herein to refer to a carrier or a diluent that does not cause significant irritation to an organism and does not abrogate the biological activity and properties of the administered compound. An adjuvant is included under these phrases.

In certain embodiments, compositions disclosed herein may further compromise one or more pharmaceutically acceptable diluent(s), excipient(s), and/or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art.

In some embodiments, pharmaceutical compositions herein may also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, or any combination thereof. Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in “Remington's Pharmaceutical Sciences,” Mack Publishing Co., Easton, Pa., latest edition, which is incorporated herein by reference.

In certain embodiments, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In some embodiments, any of the well-known techniques, carriers, and excipients may be used as suitable and/or as understood in the art.

In certain embodiments, pharmaceutical compositions described herein may be an aqueous suspension comprising one or more polymers as suspending agents. In some embodiments, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In some embodiments, pharmaceutical compositions disclosed herein may comprise at least about 5%, at least about 10%, at least about 20%, at least about 25%, at least about 30%, at least about 35%, at least about 40%, at least about 45%, or at least about 50% total amount of polymers as suspending agent(s) by total weight of the composition. In some embodiments, pharmaceutical compositions disclosed herein may comprise about 5% to about 99%, about 10%, about 95%, or about 15% to about 90% total amount of polymers as suspending agent(s) by total weight of the composition.

In certain embodiments, pharmaceutical compositions disclosed herein may comprise a viscous formulation. In some embodiments, viscosity of composition herein may be increased by the addition of one or more gelling or thickening agents. In some embodiments, compositions disclosed herein may comprise one or more gelling or thickening agents in an amount to provide a sufficiently viscous formulation to remain on treated tissue. In some embodiments, pharmaceutical compositions disclosed herein may comprise at least about 5%, at least about 10%, at least about 20%, at least about 25%, at least about 30%, at least about 35%, at least about 40%, at least about 45%, or at least about 50% total amount of gelling or thickening agent(s) by total weight of the composition. In some embodiments, pharmaceutical compositions disclosed herein may comprise about 5% to about 99%, about 10%, about 95%, or about 15% to about 90% total amount of gelling or thickening agent(s) by total weight of the composition. In some embodiments, suitable thickening agents for use herein can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or any combination thereof.

In certain embodiments, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some embodiments, pharmaceutical compositions disclosed herein may comprise at least about 5%, at least about 10%, at least about 20%, at least about 25%, at least about 30%, at least about 35%, at least about 40%, at least about 45%, or at least about 50% total amount of one or more agents by total weight of the composition. In some embodiments, pharmaceutical compositions disclosed herein may comprise about 5% to about 99%, about 10%, about 95%, or about 15% to about 90% total amount of one or more agents by total weight of the composition. In some embodiments, one or more of these agents may be added to improve the performance, efficacy, safety, shelf-life and/or other property of the muscarinic antagonist composition of the present disclosure. In some embodiments, additives may be biocompatible, without being harsh, abrasive, and/or allergenic.

In certain embodiments, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some embodiments, any pharmaceutically acceptable organic or inorganic acid may be used. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more acidifying agents by total weight of the composition. In some embodiments, pharmaceutical compositions disclosed herein may comprise about 5% to about 99%, about 10%, about 95%, or about 15% to about 90% total amount of one or more acidifying agents by total weight of the composition.

In certain embodiments, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some embodiments, any pharmaceutically acceptable organic or inorganic base can be used. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more alkalizing agents by total weight of the composition. In some embodiments, pharmaceutical compositions disclosed herein may comprise about 5% to about 99%, about 10%, about 95%, or about 15% to about 90% total amount of one or more alkalizing agents by total weight of the composition.

In certain embodiments, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate, sodium metabisulfite and other materials known to one of ordinary skill in the art. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more antioxidants by total weight of the composition. In some embodiments, pharmaceutical compositions disclosed herein may comprise about 5% to about 99%, about 10%, about 95%, or about 15% to about 90% total amount of one or more antioxidants by total weight of the composition.

In certain embodiments, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some embodiments, any pharmaceutically acceptable organic or inorganic buffer can be used. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more buffering agents by total weight of the composition. In some embodiments, pharmaceutical compositions disclosed herein may comprise about 5% to about 99%, about 10%, about 95%, or about 15% to about 90% total amount of one or more buffering agents by total weight of the composition.

In some embodiments, the amount of one or more buffering agents may depend on the desired pH level of a composition. In some embodiments, pharmaceutical compositions disclosed herein may have a pH of about 6 to about 9. In some embodiments, pharmaceutical compositions disclosed herein may have a pH greater than about 8, greater than about 7.5, greater than about 7, greater than about 6.5, or greater than about 6.

In certain embodiments, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some embodiments, any pharmaceutically acceptable preservative can be used. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more preservatives by total weight of the composition. In some embodiments, pharmaceutical compositions disclosed herein may comprise about 5% to about 99%, about 10%, about 95%, or about 15% to about 90% total amount of one or more preservatives by total weight of the composition.

In certain embodiments, pharmaceutical compositions disclosed herein may comprise one or more surface-acting reagents or detergents. In some embodiments, surface-acting reagents or detergents may be synthetic, natural, or semi-synthetic. In some embodiments, compositions disclosed herein may comprise anionic detergents, cationic detergents, zwitterionic detergents, ampholytic detergents, amphoteric detergents, nonionic detergents having a steroid skeleton, or a combination thereof. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more surface-acting reagents or detergents by total weight of the composition. In some embodiments, pharmaceutical compositions disclosed herein may comprise about 5% to about 99%, about 10%, about 95%, or about 15% to about 90% total amount of one or more surface-acting reagents or detergents by total weight of the composition.

In certain embodiments, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more stabilizers by total weight of the composition. In some embodiments, pharmaceutical compositions disclosed herein may comprise about 5% to about 99%, about 10%, about 95%, or about 15% to about 90% total amount of one or more stabilizers by total weight of the composition.

In some embodiments, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In some embodiments, a vapor pressure depression method is used to calculate the osmolarity of the compositions disclosed herein. In some embodiments, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In some embodiments, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, a pharmaceutical composition described herein may have an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more tonicity modifiers by total weight of the composition. In some embodiments, pharmaceutical compositions disclosed herein may comprise about 5% to about 99%, about 10%, about 95%, or about 15% to about 90% total amount of one or more tonicity modifiers by total weight of the composition.

### (ii) Dosage Formulations

In certain embodiments, the present disclosure provides compositions formulated for one or more routes of administration. Suitable routes of administration may, for example, include oral, rectal, transmucosal, transnasal, intestinal, and/or parenteral delivery. In some embodiments, compositions herein formulated can be formulated for parenteral delivery. In some embodiments, compositions herein formulated can be formulated intramuscular, subcutaneous, intramedullary, intravenous, intraperitoneal, and/or intranasal injections.

In certain embodiments, one may administer a composition herein in a local or systemic manner, for example, via local injection of the pharmaceutical composition directly into a tissue region of a patient. In some embodiments, a pharmaceutical composition disclosed herein can be administered parenterally, e.g., by intravenous injection, intracerebroventricular injection, intra-cisterna magna injection, intra-parenchymal injection, or a combination thereof. In some embodiments, a pharmaceutical composition disclosed herein can administered to subject as disclosed herein. In some embodiments, a pharmaceutical composition disclosed herein can administered to human patient. In some embodiments, a pharmaceutical composition disclosed herein can administered to a human patient via at least two administration routes. In some embodiments, the combination of administration routes by be intracerebroventricular injection and intravenous injection; intrathecal injection and intravenous injection; intra-cisterna magna injection and intravenous injection; and/or intra-parenchymal injection and intravenous injection.

In certain embodiments, pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

In certain embodiments, pharmaceutical compositions for use in accordance with the present disclosure thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen. For injection, the active ingredients of a pharmaceutical composition herein may be formulated in aqueous solutions, preferably in physiologically compatible buffers such as Hank's solution, Ringer's solution, physiological salt buffer, or any combination thereof.

In certain embodiments, pharmaceutical compositions described herein may be formulated in the form of a nanoparticle. The nanoparticle may have a monolayer enclosing the nanoparticle core, wherein the siRNA molecule is disposed within the nanoparticle core. In an embodiment, the nanoparticle core includes a solid lipid (i.e., lipid that remains solid at room temperature and body temperature) or a liquid lipid (i.e., oil, which remains liquid at room temperature and body temperature, for example, vegetable oil or a lipid extracted from human adipose tissue). In particular, embodiments of the present disclosure include nanoparticles and compositions for the controlled and/or sustained release (e.g., release at a predetermined rate to maintain a certain concentration for a certain period of time) of an agent, such as a small interfering RNA (siRNA) from the nanoparticle.

In certain embodiments, pharmaceutical compositions described herein may be formulated for parenteral administration, e.g., by bolus injection or continuous infusion. Formulations for injection herein may be presented in unit dosage form, e.g., in ampoules or in multidose containers with optionally, an added preservative. In some embodiments, compositions herein may be suspensions, solutions or emulsions in oily or aqueous vehicles, and/or may contain formulatory agents such as suspending, stabilizing and/or dispersing agents.

In certain embodiments, pharmaceutical compositions herein formulated for parenteral administration may include aqueous solutions of the active preparation (e.g., a siRNA molecule) in water-soluble form. In some embodiments, compositions herein comprising suspensions of the active preparation may be prepared as oily or water-based injection suspensions. Suitable lipophilic solvents and/or vehicles for use herein may include, but are not limited to, fatty oils such as sesame oil, or synthetic fatty acids esters such as ethyl oleate, triglycerides or liposomes. In some embodiments, compositions herein comprising aqueous injection suspensions may contain substances which increase the viscosity of the suspension, such as sodium carboxymethyl cellulose, sorbitol, and/or dextran. In some embodiments, compositions herein comprising a suspension may also contain one or more suitable stabilizers and/or agents which increase the solubility of the active ingredients (e.g., a siRNA molecule) to allow for the preparation of highly concentrated solutions.

In some embodiments, compositions herein may comprise the active ingredient in a powder form for constitution with a suitable vehicle, e.g., sterile, pyrogen-free water-based solution, before use.

Pharmaceutical compositions suitable for use in context of the present disclosure may include compositions wherein the active ingredients can be contained in an amount effective to achieve the intended purpose. In some embodiments, a therapeutically effective amount means an amount of active ingredients (e.g., a siRNA molecule) effective to prevent, slow, alleviate or ameliorate symptoms of a disorder (e.g., liver disease) or prolong the survival of the subject being treated.

Determination of a therapeutically effective amount is well within the capability of those skilled in the art, especially in light of the detailed disclosure provided herein.

For any preparation used in the methods of the present disclosure, the therapeutically effective amount or dose can be estimated initially from in vitro and cell culture assays and or screening platforms disclosed herein. For example, a dose can be formulated in animal models to achieve a desired concentration or titer. Such information can be used to more accurately determine useful doses in humans.

In some embodiments, toxicity and therapeutic efficacy of the active ingredients disclosed herein can be determined by standard pharmaceutical procedures in vitro, in cell cultures or experimental animals. In some embodiments, data obtained from these in vitro and cell culture assays and animal studies can be used in formulating a range of dosage for use in a human subject. In some embodiments, a dosage for use herein may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1).

In certain embodiments, dosage amounts and/or dosing intervals may be adjusted individually to brain or blood levels of the active ingredient that are sufficient to induce or suppress the biological effect (minimal effective concentration, MEC). In some embodiments, the MEC for an active ingredient (e.g., a siRNA molecule or composition disclosed herein) may vary for each preparation, but can be estimated from in vitro data. In some embodiments, dosages necessary to achieve the MEC herein may depend on individual characteristics and route of administration. Detection assays can be used to determine plasma concentrations.

In certain embodiments, depending on the severity and responsiveness of the condition to be treated, dosing with compositions herein can be of a single or a plurality of administrations, with course of treatment lasting from several days to several weeks or until cure is effected or diminution of the disease state is achieved.

In certain embodiments, amounts of a composition herein to be administered will be dependent on the subject being treated, the severity of the affliction, the manner of administration, the judgment of the prescribing physician, and the like. In some embodiments, effective doses may be extrapolated from dose-responsive curves derived from in vitro or in vivo test systems.

### III. Methods of Use

The present disclosure provides for methods of treating, attenuating, and preventing liver disease in a subject in need thereof. In several embodiments, a method for treating, attenuating, or preventing liver disease in a subject can include administering to a subject, including a human subject, an effective amount of one or more siRNA molecules targeting GPAM disclosed herein or a nucleic acid encoding an siRNA molecule targeting GPAM as disclosed herein. In several embodiments, a method for treating, attenuating, or preventing liver disease in a subject can include administering to a subject, including a human subject, an effective amount of a nucleic acid encoding a suitable sgRNA, or a suitable sgRNA targeting GPAM and an RNA guided endonuclease.

Methods disclosed herein may include treating a subject in need thereof by administrating a therapeutically effective amount of one or more siRNA molecules or a pharmaceutical composition disclosed herein. The subject may be a human subject having or suspected of having, or at risk of having liver disease, liver damage, liver dysfunction, liver injury. The term “liver disease”, “liver injury” or “liver dysfunction” may be used interchangeably and refer to any injury of the liver, including but not limited to hardening of the liver, scarring of the liver, decreased or abnormal biliary tract function, abnormal liver enzyme activity, cirrhosis of the liver, abnormal physiology as determined by common diagnostic methods include but not limited to ultrasound, or biopsy/histopathology, necrosis of the liver and the like. Non-limiting examples of liver disease to be treated using the methods disclosed herein may include fatty liver disease (FLD), alcohol-related liver disease (ARLD), non-alcoholic fatty liver disease (NAFLD), non-alcoholic steatohepatitis (NASH), end stage liver disease (cirrhosis) from any etiology, liver cancer HCC, or any combination thereof. In some embodiments, the subject benefits of an increase in life expectancy compared to an untreated subject with identical disease condition and predicted outcome. In some other embodiments, the treatment improves the subject's liver function as compared to an untreated subject with identical disease condition and predicted outcome. In yet other embodiments, the treatment attenuates the subject's liver fibrosis as compared to an untreated subject with identical disease condition and predicted outcome. In some embodiments, the treatment prevents additional liver fibrosis in the subject compared to an untreated subject with identical disease condition and predicted outcome.

A subject suitable for the liver disease treatment as disclosed herein used herein may be selected based on the subject's diagnosis. In some embodiments, a method of diagnosis may detect one or more serum markers indicative of liver disease. Non-limiting examples of serum markers indicative of a liver disease (e.g., NAFLD, or NASH) may include alpha-fetoprotein (AFP) (e.g., an AFP level of 20 ng/mL or higher), des-gamma-carboxy prothrombin, lens culinaris agglutinin-reactive AFP (AFP-L3), and the like. The diagnosis method may also include the evaluation of at least one clinical symptom associated with a liver disease. Non-limiting examples of clinical symptoms associated with a liver disease may include mild to moderate upper abdominal pain, weight loss, early satiety, or a palpable mass in the upper abdomen, paraneoplastic syndrome, hypoglycemia, erythrocytosis, hypercalcemia, intractable diarrhea and associated electrolyte disturbances (e.g., hyponatremia, hypokalemia, metabolic alkalosis), cutaneous manifestations (e.g., dermatomyositis, pemphigus foliaceus, seborrheic keratosis, pityriasis rotunda), intraperitoneal bleeding, jaundice, fever, pyogenic liver abscess, and the like. Other aspects of diagnosis may include at diagnosis and/or a determination of severity of cirrhosis, chronic infection of hepatitis B virus (HBV), chronic infection of hepatitis C virus (HCV), non-alcoholic fatty liver disease (NAFLD), non-alcoholic steatohepatitis (NASH), primary biliary cirrhosis (PBC), hereditary hemochromatosis, type 2 diabetes, obesity, tobacco use, alcohol abuse, long-term anabolic steroid use, tyrosinemia, alpha1-antitrypsin deficiency, porphyria cutanea tarda, glycogen storage diseases, Wilson disease, or any combination thereof.

In some embodiments, a subject can be diagnosed and/or predicted to have high or low risk for a liver disease (e.g., NASH or NAFLD) by histological or imaging-based examinations, such as contrast-enhanced multiphase CT, ultrasound, and/or MRI. Imaging features used to diagnose may include liver size, kinetics, and pattern of contrast enhancement, and growth on serial imaging wherein size may be measured as the maximum cross-section diameter on the image where the lesion is most clearly seen. The histologic appearance of NASH or NAFLD biopsies can include steatosis, inflammation, and fibrosis.

The methods and compositions of the present disclosure are useful for the treatment of subjects having fatty liver related disorders, such as NAFLD and/or NASH. The subject may have normal or substantially normal biliary tract function. The presence of normal or abnormal biliary tract function may be determined in a subject using any suitable methods known in the art. Generally, preferred tests for biliary tract function in NASH patients may be characterized in two groups: physiological based tests and biochemical based tests. Physiological based tests may include but are not limited to abdominal ultrasound, abdominal CT scan, endoscopic retrograde cholangiopancreatography (ECRP), Percutaneous transhepatic cholangiogram (PTCA) or Magnetic resonance cholangiopancreatography (MRCP). Biochemical based tests may include but are not limited to GGT tests, liver function tests, bilirubin tests, alkaline phosphatase (ALP) tests, liver enzyme tests, amylase blood test, lipase blood test, prothrombin time, and measurement of urine bilirubin. In some cases, one or more tests may be used to characterize biliary function. In some cases, a combination of tests may be used to assess biliary function in NASH subjects.

In some embodiments, any of the methods disclosed herein can further include monitoring occurrence of one or more adverse effects in the subject. Adverse effects may include, but are not limited to, hepatic impairment, hematologic toxicity, neurologic toxicity, cutaneous toxicity, gastrointestinal toxicity, or a combination thereof. When one or more adverse effects are observed, the methods disclosed herein can further include reducing or increasing the dose of one or more of the treatment regimens depending on the adverse effect or effects in the subject. For example, when a moderate to severe hepatic impairment is observed in a subject after treatment, compositions of use to treat the subject can be reduced in concentration or frequency.

In certain embodiments, treatments administered according to the methods disclosed herein can improve patient life expectancy compared to the life expectancy of an untreated subject with identical disease condition (e.g., NAFLD or NASH) and predicted outcome. As used herein, “patient life expectancy” is defined as the time at which 50 percent of subjects are alive and 50 percent have passed away. In some embodiments, patient life expectancy can be indefinite following treatment according to the methods disclosed herein. In other aspects, patient life expectancy can be increased at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In some embodiments, patient life expectancy can be increased at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In some embodiments, patient life expectancy can be increased at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated patient with identical disease condition and predicted outcome.

In some embodiments, treatment of a liver disease, such as NAFLD or NASH, according to the methods disclosed herein can result in attenuating, shrinking, reducing or preventing of a liver fibrosis in comparison to the starting size of the liver fibrosis. In some embodiments, attenuating or shrinking an area of liver fibrosis may be at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% (meaning that the liver fibrosis is completely gone after treatment) compared to the starting size of the liver fibrosis.

In some embodiments, treatment of a liver disease, such as NAFLD or NASH, according to the methods disclosed herein can result in an improved liver function. Liver function may be tested through routine biochemical methods. Biochemical tests may include but are not limited to GGT tests, liver function tests, bilirubin tests, alkaline phosphatase (ALP) tests, liver enzyme tests, amylase blood test, lipase blood test, prothrombin time, and measurement of urine bilirubin. In some cases, one or more tests may be used to characterize biliary function. In some cases, a combination of tests may be used to assess biliary functions. The liver function improvement can also be assessed by subject's symptom relief, such as relieving in fatigue, weight loss, and weakness of the subject. Other relieved symptoms may include reduced fluid retention, muscle wasting, bleeding from the intestines, and any combination thereof.

### IV. Kits

The present disclosure provides kits for using the methods disclosed herein. In some aspects, the present disclosure provides a kit for treating a liver disease (e.g., NASH or NAFLD) as disclosed herein and for diagnosing the liver disease. Such a kit may comprise a means for holding and/or administering such a siRNA composition or a pharmaceutical composition.

In some embodiments, kits disclosed herein can have a medical container, which holds the composition in a safe, stable and durable way. In some examples, kits disclosed herein may also comprise a means to administer the composition, such as a needle or a spatula.

Any of the kits may further comprise an instruction manual providing guidance for using the kit for treatment. The manual may be written with the physician or the liver specialist as the intended reader.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1

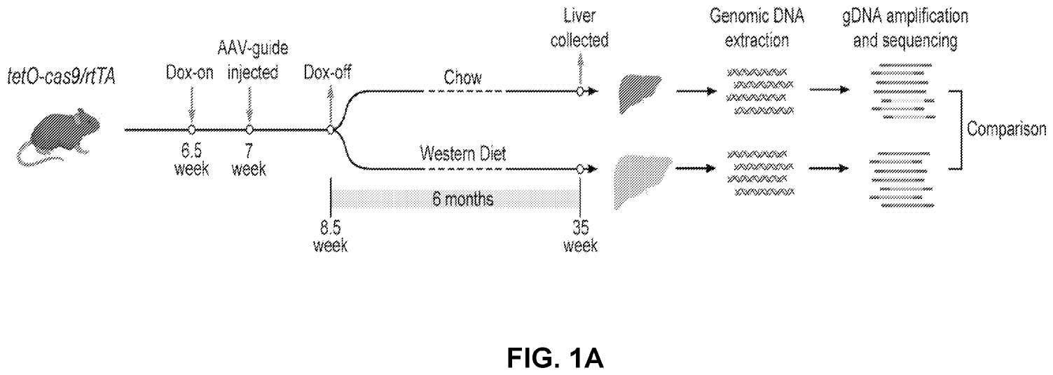

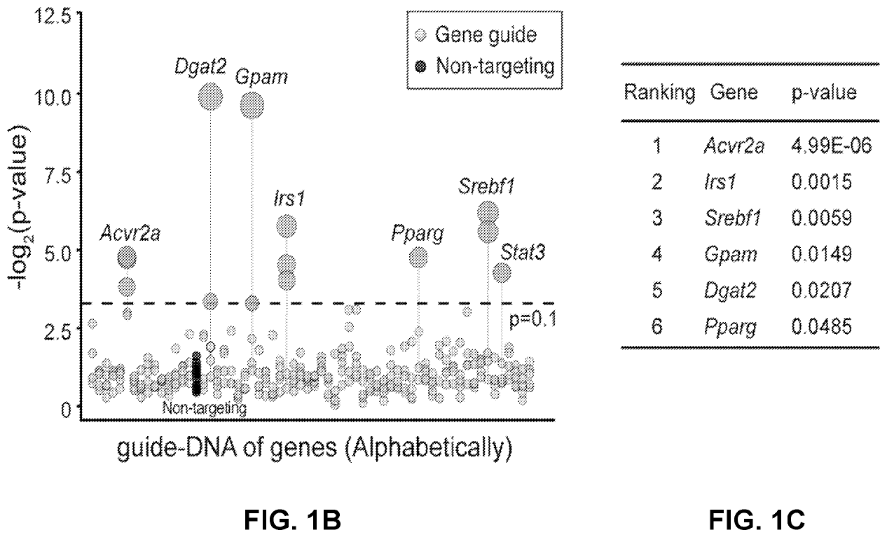

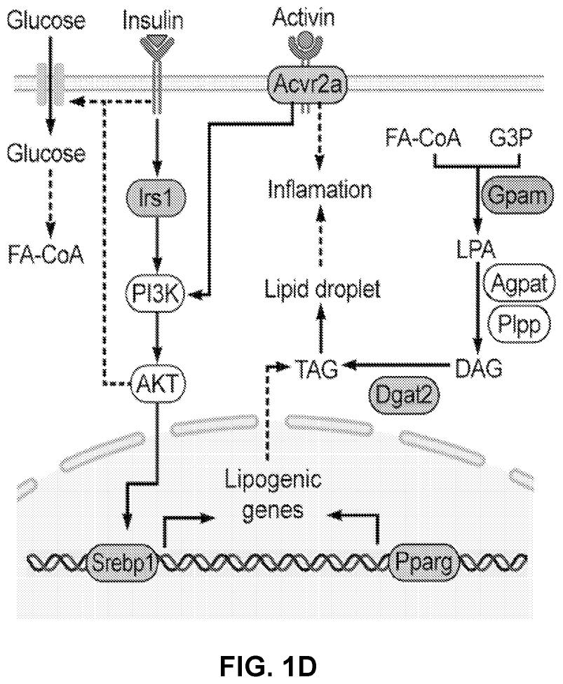

To broadly understand how mutations in NASH candidate genes influence clonal competition in an unbiased fashion in vivo, a method was developed to generate pools of mutant cells within the tissues of Cas9-expressing mice. To generate a virus that can integrate into the genome and thus permit in vivo screening, a hybrid Adeno-Associated Virus (AAV) was created that carries sleeping beauty transposase (SB100), Cre, single guide (sg)RNAs, and whose payload was flanked by transposon integration sequences (SB-IR). This AAV was used to deliver sgRNAs into the livers of dox-inducible Cas9-expressing mice for in vivo CRISPR screening. In brief, a pooled screening of 63 NASH genes identified in GWAS (i.e. APOB, TM6SF2, GCKR), exome-seq (i.e. PNPLA3, MBOAT7, HSD17B13), somatic mutation sequencing efforts (i.e. PPARGC1B, FOXO1, GPAM, ACVR2A) or biochemical studies was performed. Induction of Cas9 after AAV injection established a pool of hepatocytes with mosaic deletion of these NASH genes. After two weeks of Cas9 activity, dox was discontinued so no further cutting would occur. These mosaic mice were then given normal chow (NC) or western diet (WD: high sugar, fat, cholesterol) to understand clonal dynamics in both conditions. After 6 months, deep sequencing of sgRNAs pinpointed the genes that had the largest effects on clonal fitness, specifically in the NASH setting. To exclude purely proliferative effects that would occur independent of NASH, enriched or depleted sgRNAs that were shared in NC and WD conditions were excluded. Six genes, when deleted, were most associated with clonal expansion: Acvr2a, Irs1, Srebp1, Gpam, Dgat2, and Pparg (FIGS. 1A-1D).

Glycerol-3-Phosphate Acyltransferase (GPAT), including GPAT1 and GPAM (Glycerol-3-Phosphate Acyltransferase, Mitochondrial), catalyzes the initial and committing step in glycerolipid biosynthesis, and was predicted to play a pivotal role in the regulation of cellular triacylglycerol and phospholipid levels. It is noted that GPAM mutant clones were more competitive and exert a beneficial effect in NAFLD/NASH. For example, in NASH livers recurrent and convergent loss of function mutations in genes were identified that promote lipogenesis (Ng et. al., Nature 2021). The abnormal accumulation of lipid droplets in hepatocytes can eventually lead to lipotoxicity, cell death, and cirrhosis. The observation of loss of function mutations in metabolic enzymes that generate hepatic lipids (such as GPAM) suggest that some somatic mutations can confer increased fitness through a reversal of the driving etiology of disease.

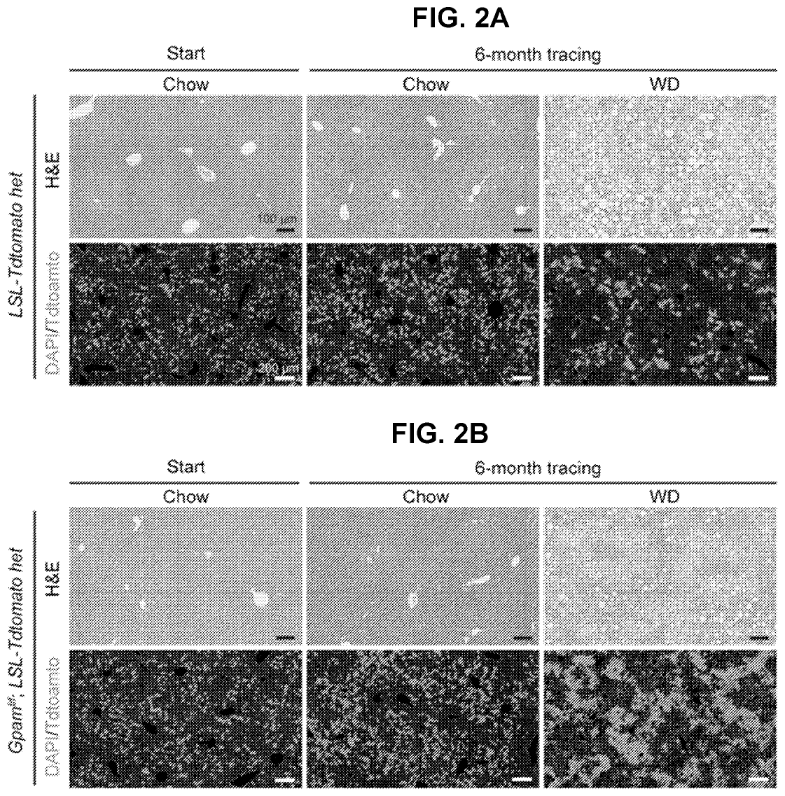

Top hits from in vivo NASH mouse screens were analyzed in mosaic liver disease models. In order to study GPAM mutations in the mosaic settings with liver specific deletion or clonal mosaicism, CRISPR approaches were used to generate both whole body GPAM Knockout mice and GPAM floxed mice targeting exon 3, which is predicted to lead to frameshift and stop codon causing early termination. It was confirmed that GPAM floxed mice achieved GPAM deletion after Cre recombination. To study how mosaic GPAM Wild Type and Knockout clones in the liver compete under normal and NAFLD conditions, low dose AAV-Cre was given to induce deletion in 10-20% of liver cells, then mosaic mice were put on normal chow or WD conditions. This resulted in increased survival of GPAM Knockout clones after 6 months and reduced NASH severity (FIGS. 2A-2B). This data mirrored the human genetic data which showed that GPAM mutant clones are selected for in human livers with NASH.

To further ascertain which genes are increasing clonal expansion through an influence on metabolic fitness, liver specific GPAM Knockout mice were used to assess the extent to which ideal siRNA suppression of this gene affected liver and whole body metabolism in the context of NASH. For each conditional CRISPR KO model, body weight, liver weight, histology, steatosis, fibrosis, serum tests (liver function tests, cholesterol, TGs, non-esterified free fatty acids (NEFAs)) were measured. After 3 months of NASH diets, liver specific GPAM knockout mice were protected from liver triglyceride accumulation.

### Example 2

In order to address if a mouse siRNA against GPAM may prevent and/or reverse NASH in a mouse model, an siRNA tool compound used in mice is used to study translational aspects of GPAM biology. Eight siRNAs were tested against mouse GPAM in Cos7 cells to identify the siRNA sequence with the highest knockdown efficiency based on mouse GPAM reporter assays. Then this siRNA was modified and conjugated with GalNAc. Briefly, in order to minimize nucleolytic degradation and immune responses in vivo, every 2′-position was modified with 2′-O-methyl. To determine if these stabilizing modifications influence silencing activities, siRNA activity in vitro prior to GalNAc conjugation was determined. In accord with the industry standard approach, GalNAc conjugation may allow high efficiency delivery to hepatocytes without lipid nanoparticle packaging. the best in vivo dosing regimen was determined for GalNAc-siGPAM. Three doses of siRNAs in PBS (1, 5, 10, 15 mg/kg) were given subcutaneously (SC), once every two weeks starting at 4 weeks of age to WT B6 mice. After siRNA treatments, mice were sacrificed for GPAM qPCR, western blotting, and histology (n=5 mice/group). Maximum tolerated dose was determined based on lack of severe toxicities such as weight loss greater than 10%, organ (heart/kidney/liver) failure, and death. In all of the following siRNA experiments, GalNAc-siLuc serves as the control.

To determine if GalNAc-siGPAM prevents NASH, the optimized dose defined above was used. Then it was determined if GalNAc-siRNA can mimic the GPAM KO model. In a parallel fashion as the genetic experiments, GalNAc-siRNA SC injections were started at 6 weeks of age and NASH diets were started at 8 weeks and continued for 12 weeks (6 total siRNA doses). Mice were euthanized at 20 weeks of age, a time point at which steatosis, inflammation, and fibrosis were accessed. Without being bound by theory, the siRNA approach mimics the genetic NASH prevention model.

To determine if NASH can be reversed by GalNAc-siGPAM, siRNA dosing was initiated in mice that had already received 24 weeks of WD diets (30 weeks of age) and the WD and siRNAs were continued for 12 weeks total. Then the livers were assessed for pathological features of NASH. Assessment includes hepatocyte ballooning, inflammation, and fibrosis, features that characterize NASH as described above. These siRNA experiments, provide immense translational value and pave the path toward clinical therapeutics.

### Example 3

Specific siRNA sequences were designed to target human GPAM for the treatment of NASH. Based on the human somatic sequencing data which has identified recurrent loss of function GPAM mutations in NASH livers, screening genetic screening results according to the exemplary methods herein, and the biology of GPAM in mouse models of NASH and insulin resistance, GPAM represents a promising therapeutic target for human NASH.

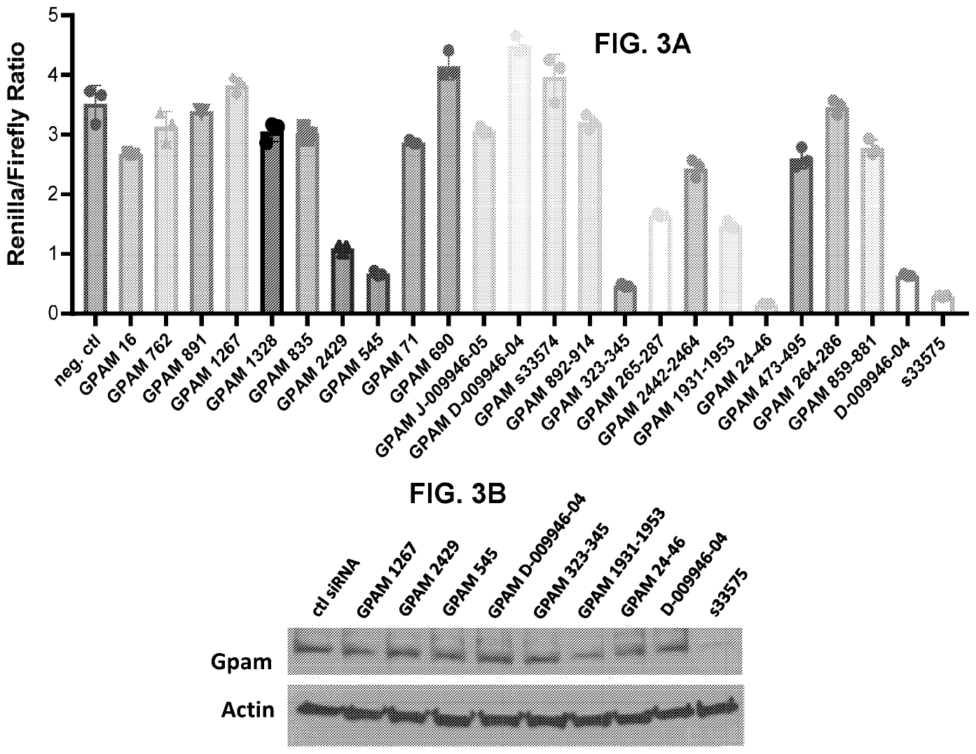

Tests identified optimized siRNA sequences corresponding to the human sequences of human GPAM. This involved screening many candidate siRNAs per gene target using in vitro luciferase reporter-based assays. In brief, full length GPAM cDNA (without a 3′UTR) was cloned into a psicheck2 plasmid backbone containing a luciferase gene. This created a Renilla luciferase-GPAM fusion gene. The luciferase assay is a dual reporter system with Firefly luciferase as a control and Renilla luciferase as the read out for transcription and translation. Thus, the ratio of Renilla to Firefly signal accounts for variations in transfection efficiency and cell viability. The assay was carried out in a 96 well plate format with technical replicates using a 48 hour time point for the assay readout. The reporter along with siRNAs were co-transfected into Cos7 monkey kidney cells using lipofectamine. After 48 hours, the cells were lysed and the signal was captured by a luminometer using the substrates for Firefly and Renilla. Effective siRNAs against a target gene showed a reduced ratio for Renilla vs. Firefly signal compared to non-targeting controls. A total of 39 siRNAs targeting the entire GPAM ORF were designed and tested (FIGS. 3A-3B). At least 21 effective siRNAs were identified against human GPAM (Table 3). Optimized siRNAs are modified by conjugating to acetylgalactosamine (GalNAC) in the standard fashion and tested in vivo.

TABLE 3

| | % mRNA remaining | Experimental |
|---|---|---|
| | (compared to neg. | Design or |
| | ctl siRNA, at | Manufacturer |
| siRNA ID | 3.3 nM or 0.5 pmol) | Design |
| GPAM 16 | 76.3 | Experimental |
| GPAM 762 | 89.0 | Experimental |
| GPAM 891 | 96.4 | Experimental |
| GPAM 1267 | 108.6 | Experimental |
| GPAM 1328 | 86.8 | Experimental |
| GPAM 835 | 85.8 | Experimental |
| GPAM 2429 | 31.2 | Experimental |
| GPAM 545 | 19.0 | Experimental |
| GPAM 71 | 81.5 | Experimental |
| GPAM 690 | 117.8 | Experimental |
| GPAM 892-914 | 91.0 | Experimental |
| GPAM 323-345 | 13.3 | Experimental |
| GPAM 265-287 | 46.9 | Experimental |
| GPAM 2442-2464 | 69.2 | Experimental |
| GPAM 1931-1953 | 41.8 | Experimental |
| GPAM 24-46 | 4.6 | Experimental |
| GPAM 473-495 | 74.0 | Experimental |
| GPAM 264-286 | 98.2 | Experimental |
| GPAM 859-881 | 78.9 | Experimental |
| J-009946-05 | 21.4 | Dharmacon |
| J-009946-06 | 22.8 | Dharmacon |
| J-009946-07 | 24.9 | Dharmacon |
| J-009946-08 | 24.0 | Dharmacon |
| D-009946-01 | 20.7 | Dharmacon |
| D-009946-02 | 45.6 | Dharmacon |
| D-009946-03 | 21.2 | Dharmacon |
| D-009946-04 | 18.2 | Dharmacon |
| s224483 | 18.0 | ThermoFisher |
| s224484 | 14.6 | ThermoFisher |
| s33574 | 20.6 | ThermoFisher |
| s33575 | 8.4 | ThermoFisher |
| s33576 | 29.2 | ThermoFisher |
| 112114 | 15.0 | ThermoFisher |
| 112115 | 43.1 | ThermoFisher |
| 112116 | 71.7 | ThermoFisher |
| 117624 | 26.3 | ThermoFisher |
| 43864 | 15.0 | ThermoFisher |
| 43960 | 15.7 | ThermoFisher |
| 44047 | 15.4 | ThermoFisher |

### Example 4

Some of the human siRNAs against GPAM disclosed herein are tested in human cell line models of NASH. The tests are performed using optimized siRNAs and/or their conjugates with acetylgalactosamine (GalNAc) to improve liver targeting. The experimental design has three steps. First, use the identified siRNAs in human liver cancer cells, such as Huh7 or HepG2, to knockdown target genes of interest, i.e. GPAM. Specifically, Huh7 cells are grown to 60-80% confluence and then transfected with siRNAs. Opti-MEM (Thermo Fisher 31985062) containing the siRNAs and Lipofectamine are combined, incubated, and then added to cells for transfection (400 μl/well to a 6-well plate or 2.4 ml to a 100 mm plate). Transfection medium is replaced after 6 h with medium containing 100 μM FA. Then, feed cells lipids and label with lipid dyes. Lastly, perform fluorescence-activated cell sorting (FACS) to quantitate lipid accumulation. FACS to purify specific cell populations based on phenotypes detected by flow cytometry. This method enables characterization of a single cell population without the influence of other cells. Cells are stained with 1 ml PBS containing 10 μg of BODIPY 493/503 (BD) (Thermo Fisher D3922) for 15 min at room temperature, then subject to FACs measurements. Early studies show that in lipophilic fluorophore stained cells, fluorescence intensity measured by FC reflects lipid levels (Wolins, 2018).

An aliquot of the cells grown under each condition is assayed for triacylglycerol (TG). In brief, cells are trypsinized, washed once with PBS, resuspended in PBS with 10 mM EDTA, and then counted. Cells are divided in triplicate into 13×100 mm glass tubes in a final volume of 200 μl. Triolein standards (Sigma T7140) are also prepared in a final volume of 200 μl PBS/10 mM EDTA in 13×100 mm glass tubes. TGs are extracted and quantified. Briefly, 2 ml of isopropanol:hexane:water (40:10:1) is added to cells or standards and the samples are vortexed, covered, and incubated at room temperature for 30 min. Then, 500 μl of a 1:1 mixture of hexane:diethylether is added to the samples followed by vortexing and incubating for an additional 10 min at room temperature. Next, 1 ml of water is added to samples, tubes are vortexed, and layers are allowed to separate at room temperature while covered for 30-45 min. Using Pasteur pipettes, the top layer is transferred to 12×75 mm glass tubes and dried under N₂ to completion. Following the drying step, 400 μl of Infinity triglyceride reagent (Thermo Scientific TR22421) is added to each tube and vortexed. Tubes are covered and incubated for 90 min at 37° C. with shaking at 250 rpm. Finally, 300 μl of each sample is transferred to 96-well plates and absorbance is measured at 540 nm using a microplate reader. To determine whether fixing cells affected the TG measurement, TG is quantified in fixed and unfixed cells from the same batch of Huh7 cells incubated in 2 mM FA.

### Example 5

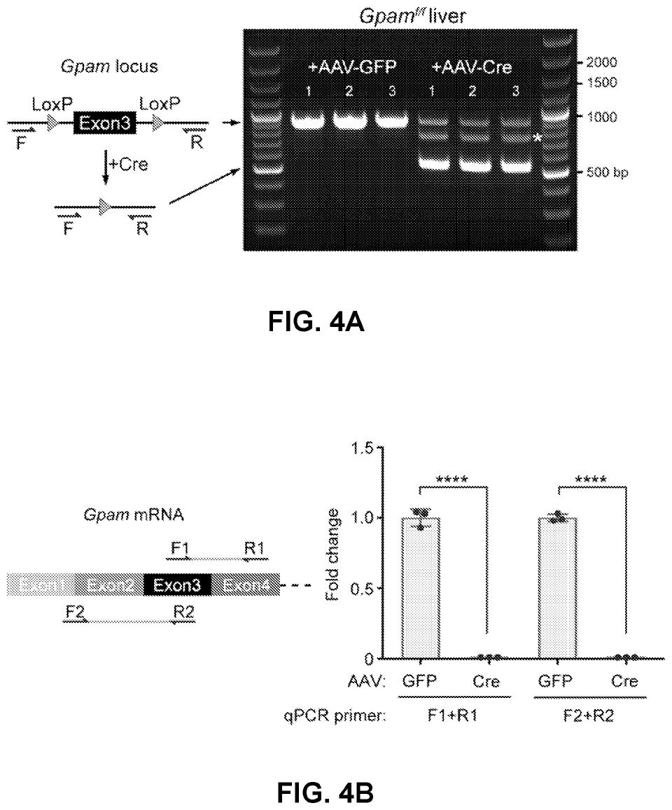

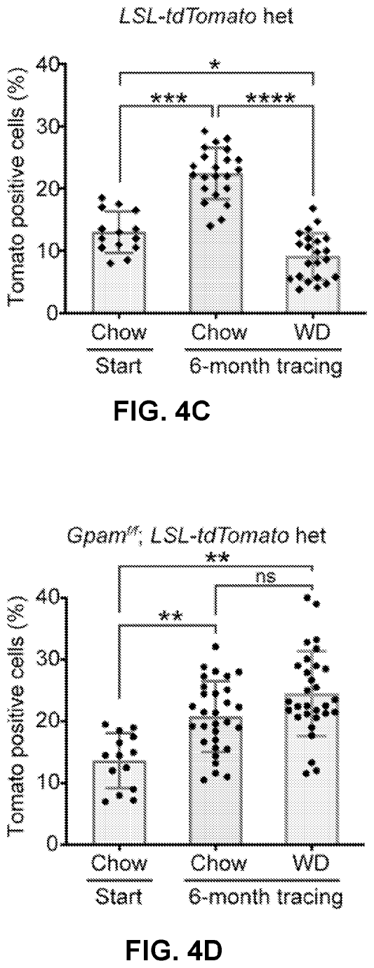

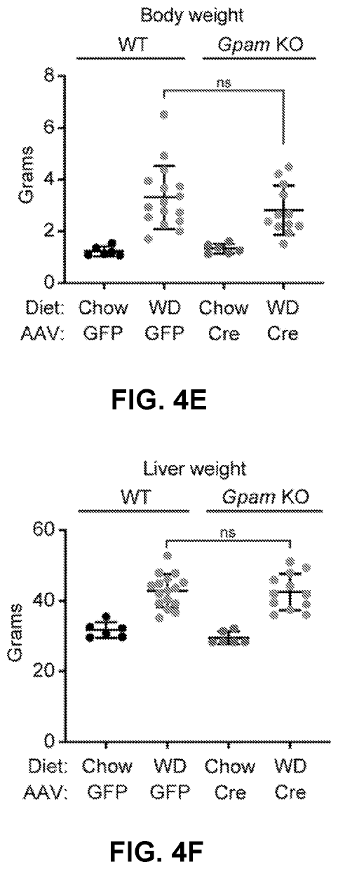

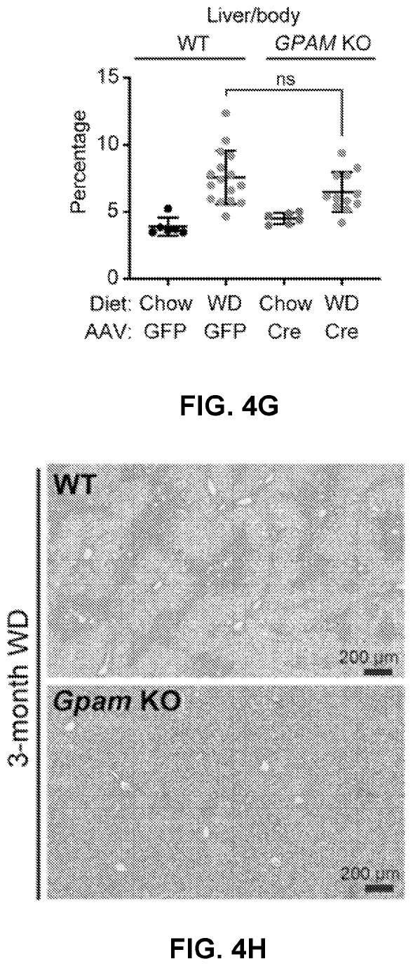

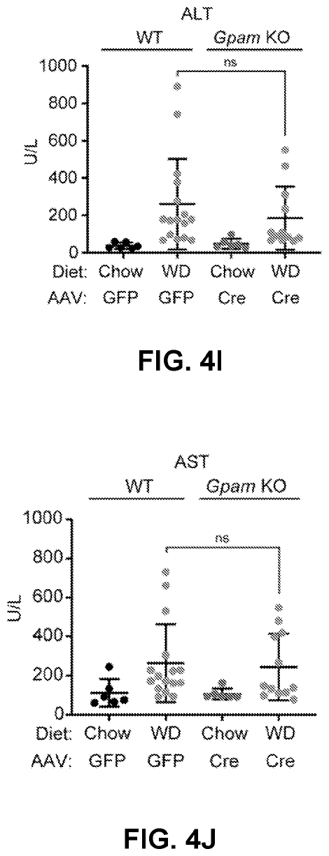

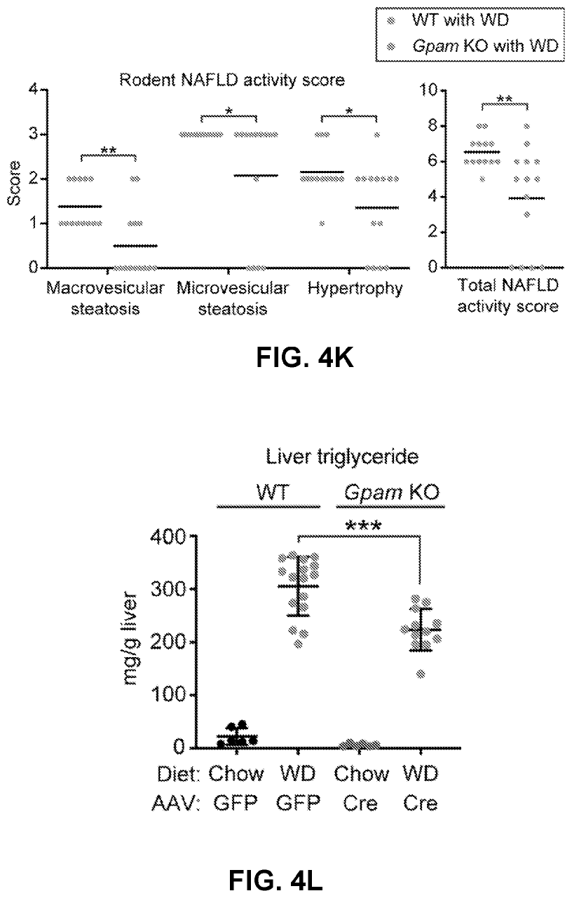

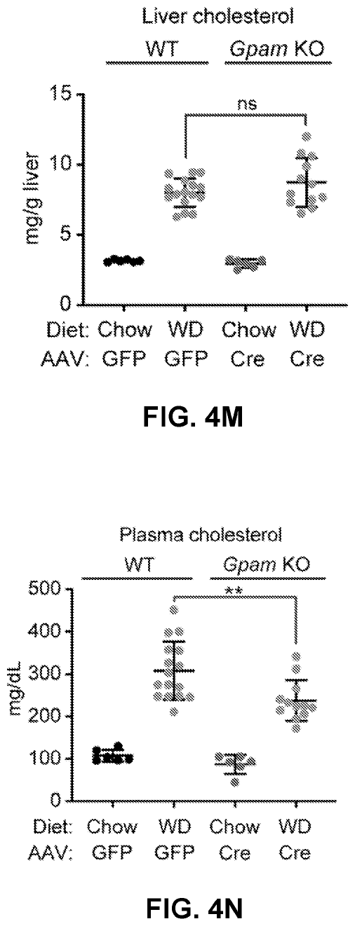

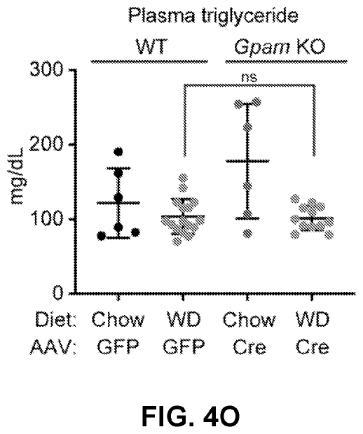

Mutations in Gpam were further investigated, because it appeared as a top hit from the functional NASH screen described above and from human somatic mutation sequencing efforts. GPAM catalyzes the acylation of glycerol-3-phosphate with acyl-coenzyme A (CoA) to generate CoA and lysophosphatidic acid (LPA), the rate-limiting step in triacylglycerol synthesis. Because conditional KO mice required to study clonal dynamics were not available, GPAM floxed mice targeting exon 3, whose deletion is predicted to lead to a frameshift and a premature stop codon were generated as provided in Example 2. Saturating doses of AAV8-TBG-Cre led to the deletion of exon 3 and almost complete depletion of GPAM mRNA one week after injection (FIGS. 4A and 4B). Using these floxed mice, WT and KO clones were compared under normal and WD conditions. An increase in abundance of GPAM KO clones in the setting of western diet (WD) was observed (FIGS. 4C and 4D). These data demonstrate the diet and steatosis dependent beneficial effects of GPAM loss of function mutations.

To determine why GPAM mutant clones were more fit, whole-liver of GPAM KO mice were induced with AAV8-TBG-Cre. Modest body and liver weight differences were observed after 3 months of WD (FIGS. 4E and 4F, Table 4), but KO mice showed a trend toward decreased liver/body weight ratios (FIG. 4G), decreased steatosis (FIG. 4H), and a trend toward improved transaminitis (FIGS. 41 and 4J).

TABLE 4

| | | | | | | | | | liver- | liver- |
|---|---|---|---|---|---|---|---|---|---|---|
| | | | | liver/ | | | plasma- | plasma- | Cholesterol | Triglyceride |
| | | body | liver | body | AST | ALT | Cholesterol | Triglyceride | (mg/gram | (mg/gram |
| # | treatment | (gram) | (gram) | (%) | (U/L) | (U/L) | (mg/dL) | (mg/dL) | liver) | liver) |
| 4656 | Gpam7f/f- | 32.23 | 1.12 | 3.48 | 134 | 26 | 96 | 162.2 | 3.07 | 13.66 |
| | satuated | | | | | | | | | |
| | AAV- | | | | | | | | | |
| | GFP, | | | | | | | | | |
| | chow | | | | | | | | | |
| 4657 | Gpam7f/f- | 29.70 | 1.08 | 3.64 | 76 | 35 | 104 | 83 | 3.15 | 11.57 |
| | satuated | | | | | | | | | |
| | AAV- | | | | | | | | | |
| | GFP, | | | | | | | | | |
| | chow | | | | | | | | | |
| 4658 | Gpam7f/f- | 30.68 | 1.07 | 3.49 | 66 | 26 | 97 | 77.9 | 3.14 | 14.17 |
| | satuated | | | | | | | | | |
| | AAV- | | | | | | | | | |
| | GFP, | | | | | | | | | |
| | chow | | | | | | | | | |
| 4659 | Gpam7f/f- | 32.62 | 1.21 | 3.71 | 61 | 23 | 120 | 89.5 | 3.30 | 40.05 |
| | satuated | | | | | | | | | |
| | AAV- | | | | | | | | | |
| | GFP, | | | | | | | | | |
| | chow | | | | | | | | | |
| 4713 | Gpam7f/f- | 35.56 | 1.37 | 3.85 | 92 | 60 | 130 | 129.5 | 3.07 | 44.83 |
| | satuated | | | | | | | | | |
| | AAV- | | | | | | | | | |
| | GFP, | | | | | | | | | |
| | chow | | | | | | | | | |
| 4721 | Gpam7f/f- | 29.57 | 1.55 | 5.24 | 246 | 57 | 99 | 190.6 | 3.26 | 8.24 |
| | satuated | | | | | | | | | |
| | AAV- | | | | | | | | | |
| | GFP, | | | | | | | | | |
| | chow | | | | | | | | | |
| 4639 | Gpam7f/f- | 42.92 | 3.94 | 9.18 | 306 | 376 | 275 | 98 | 7.69 | 363.53 |
| | satuated | | | | | | | | | |
| | AAV- | | | | | | | | | |
| | GFP, | | | | | | | | | |
| | WD | | | | | | | | | |
| 4640 | Gpam7f/f- | 47.75 | 4.93 | 10.32 | 530 | 742 | 400 | 93.5 | 8.53 | 322.58 |
| | satuated | | | | | | | | | |
| | AAV- | | | | | | | | | |
| | GFP, | | | | | | | | | |
| | WD | | | | | | | | | |
| 4648 | Gpam7f/f- | 36.70 | 1.71 | 4.66 | 93 | 73 | 269 | 122.9 | 7.36 | 265.58 |
| | satuated | | | | | | | | | |
| | AAV- | | | | | | | | | |
| | GFP, | | | | | | | | | |
| | WD | | | | | | | | | |
| 4649 | Gpam7f/f- | 40.33 | 2.40 | 5.95 | 161 | 177 | 248 | 87.1 | 6.51 | 336.86 |
| | satuated | | | | | | | | | |
| | AAV- | | | | | | | | | |
| | GFP, | | | | | | | | | |
| | WD | | | | | | | | | |
| 4650 | Gpam7f/f- | 44.88 | 3.13 | 6.97 | 173 | 173 | 211 | 98.8 | 8.87 | 273.39 |
| | satuated | | | | | | | | | |
| | AAV- | | | | | | | | | |
| | GFP, | | | | | | | | | |
| | WD | | | | | | | | | |
| 4651 | Gpam7f/f- | 42.02 | 2.78 | 6.62 | 228 | 278 | 355 | 98.1 | 8.38 | 326.90 |
| | satuated | | | | | | | | | |
| | AAV- | | | | | | | | | |
| | GFP, | | | | | | | | | |
| | WD | | | | | | | | | |
| 4652 | Gpam7f/f- | 45.22 | 3.36 | 7.43 | 163 | 180 | 247 | 70.5 | 8.32 | 357.77 |
| | satuated | | | | | | | | | |
| | AAV- | | | | | | | | | |
| | GFP, | | | | | | | | | |
| | WD | | | | | | | | | |
| 4653 | Gpam7f/f- | 47.90 | 3.73 | 7.79 | 196 | 178 | 248 | 82.2 | 6.29 | 343.61 |
| | satuated | | | | | | | | | |
| | AAV- | | | | | | | | | |
| | GFP, | | | | | | | | | |
| | WD | | | | | | | | | |
| 4654 | Gpam7f/f- | 43.64 | 2.54 | 5.82 | 94 | 67 | 246 | 89.3 | 7.86 | 222.10 |
| | satuated | | | | | | | | | |
| | AAV- | | | | | | | | | |
| | GFP, | | | | | | | | | |
| | WD | | | | | | | | | |
| 4684 | Gpam7f/f- | 37.59 | 2.94 | 7.82 | 171 | 158 | 326 | 115.9 | 7.68 | 360.59 |
| | satuated | | | | | | | | | |
| | AAV- | | | | | | | | | |
| | GFP, | | | | | | | | | |
| | WD | | | | | | | | | |
| 4685 | Gpam7f/f- | 52.76 | 6.52 | 12.36 | 731 | 891 | 451 | 155.7 | 9.45 | 320.45 |
| | satuated | | | | | | | | | |
| | AAV- | | | | | | | | | |
| | GFP, | | | | | | | | | |
| | WD | | | | | | | | | |
| 4689 | Gpam7f/f- | 44.25 | 3.67 | 8.29 | 224 | 205 | 357 | 122.7 | 8.05 | 356.20 |
| | satuated | | | | | | | | | |
| | AAV- | | | | | | | | | |
| | GFP, | | | | | | | | | |
| | WD | | | | | | | | | |
| 4707 | Gpam7f/f- | 46.17 | 4.38 | 9.49 | 663 | 422 | 397 | 115.8 | 9.35 | 333.21 |
| | satuated | | | | | | | | | |
| | AAV- | | | | | | | | | |
| | GFP, | | | | | | | | | |
| | WD | | | | | | | | | |
| 4716 | Gpam7f/f- | 39.32 | 2.26 | 5.75 | 135 | 80 | 304 | 77.8 | 7.89 | 196.0 |
| | | | | | | | | | | 1 |
| | satuated | | | | | | | | | |
| | AAV- | | | | | | | | | |
| | GFP, | | | | | | | | | |
| | WD | | | | | | | | | |
| 4717 | Gpam7f/f- | 35.24 | 2.00 | 5.68 | 228 | 67 | 319 | 91.6 | 9.45 | 215.20 |
| | satuated | | | | | | | | | |
| | AAV- | | | | | | | | | |
| | GFP, | | | | | | | | | |
| | WD | | | | | | | | | |
| 4770 | Gpam7f/f- | 39.28 | 2.73 | 6.95 | 115 | 97 | 275 | 142.5 | 6.46 | 285.81 |
| | satuated | | | | | | | | | |
| | AAV- | | | | | | | | | |
| | GFP, | | | | | | | | | |
| | WD | | | | | | | | | |
| 4599 | Gpam7f/f- | 32.12 | 1.61 | 5.01 | 105 | 44 | 82 | 144.9 | 2.69 | 3.89 |
| | satuated | | | | | | | | | |
| | AAV- | | | | | | | | | |
| | Cre, | | | | | | | | | |
| | chow | | | | | | | | | |
| 4634 | Gpam7f/f- | 28.27 | 1.38 | 4.88 | 93 | 31 | 45 | 254.4 | 2.52 | 3.67 |
| | satuated | | | | | | | | | |
| | AAV- | | | | | | | | | |
| | Cre, | | | | | | | | | |
| | chow | | | | | | | | | |
| 4635 | Gpam7f/f- | 28.33 | 1.16 | 4.09 | 94 | 37 | 92 | 107.7 | 2.95 | 4.25 |
| | satuated | | | | | | | | | |
| | AAV- | | | | | | | | | |
| | Cre, | | | | | | | | | |
| | chow | | | | | | | | | |
| 4714 | Gpam7f/f- | 28.31 | 1.13 | 3.99 | 93 | 26 | 105 | 81.5 | 3.26 | 8.64 |
| | satuated | | | | | | | | | |
| | AAV- | | | | | | | | | |
| | Cre, | | | | | | | | | |
| | chow | | | | | | | | | |
| 4720 | Gpam7f/f- | 28.37 | 1.25 | 4.41 | 92 | 57 | 94 | 257.1 | 3.19 | 10.02 |
| | satuated | | | | | | | | | |
| | AAV- | | | | | | | | | |
| | Cre, | | | | | | | | | |
| | chow | | | | | | | | | |
| 4722 | Gpam7f/f- | 31.33 | 1.48 | 4.72 | 162 | 98 | 105 | 223.5 | 3.14 | 5.71 |
| | satuated | | | | | | | | | |
| | AAV- | | | | | | | | | |
| | Cre, | | | | | | | | | |
| | chow | | | | | | | | | |
| 4604 | Gpam7f/f- | 44.17 | 3.41 | 7.72 | 266 | 233 | 266 | 127.7 | 7.93 | 275.06 |
| | satuated | | | | | | | | | |
| | AAV- | | | | | | | | | |
| | Cre, | | | | | | | | | |
| | WD | | | | | | | | | |
| 4636 | Gpam7f/f- | 39.44 | 2.38 | 6.03 | 78 | 67 | 233 | 91 | 7.67 | 213.45 |
| | satuated | | | | | | | | | |
| | AAV- | | | | | | | | | |
| | Cre, | | | | | | | | | |
| | WD | | | | | | | | | |
| 4637 | Gpam7f/f- | 51.00 | 4.22 | 8.27 | 419 | 465 | 312 | 100.1 | 10.80 | 281.76 |
| | satuated | | | | | | | | | |
| | AAV- | | | | | | | | | |
| | Cre, | | | | | | | | | |
| | WD | | | | | | | | | |
| 4638 | Gpam7f/f- | 49.35 | 3.82 | 7.74 | 480 | 552 | 241 | 89.7 | 10.60 | 263.50 |
| | satuated | | | | | | | | | |
| | AAV- | | | | | | | | | |
| | Cre, | | | | | | | | | |
| | WD | | | | | | | | | |
| 4646 | Gpam7f/f- | 41.96 | 2.28 | 5.43 | 98 | 80 | 225 | 96.4 | 7.33 | 231.32 |
| | satuated | | | | | | | | | |
| | AAV- | | | | | | | | | |
| | Cre, | | | | | | | | | |
| | WD | | | | | | | | | |
| 4647 | Gpam7f/f- | 36.04 | 1.51 | 4.19 | 148 | 63 | 172 | 122.6 | 6.53 | 140.01 |
| | satuated | | | | | | | | | |
| | AAV- | | | | | | | | | |
| | Cre, | | | | | | | | | |
| | WD | | | | | | | | | |
| 4683 | Gpam7f/f- | 35.98 | 2.21 | 6.14 | 139 | 108 | 206 | 80.3 | 7.50 | 194.97 |
| | satuated | | | | | | | | | |
| | AAV- | | | | | | | | | |
| | Cre, | | | | | | | | | |
| | WD | | | | | | | | | |
| 4687 | Gpam7f/f- | 47.81 | 4.49 | 9.39 | 399 | 310 | 341 | 115.7 | 9.89 | 235.10 |
| | satuated | | | | | | | | | |
| | AAV- | | | | | | | | | |
| | Cre, | | | | | | | | | |
| | WD | | | | | | | | | |
| 4715 | Gpam7f/f- | 41.87 | 2.65 | 6.33 | 133 | 90 | 215 | 79.5 | 9.02 | 205.87 |
| | satuated | | | | | | | | | |
| | AAV- | | | | | | | | | |
| | Cre, | | | | | | | | | |
| | WD | | | | | | | | | |
| 4718 | Gpam7f/f- | 39.05 | 2.19 | 5.61 | 548 | 109 | 221 | 91.5 | 12.01 | 219.77 |
| | satuated | | | | | | | | | |
| | AAV- | | | | | | | | | |
| | Cre, | | | | | | | | | |
| | WD | | | | | | | | | |
| 4719 | Gpam7f/f- | 45.89 | 2.67 | 5.82 | 114 | 85 | 195 | 110.3 | 8.62 | 195.43 |
| | satuated | | | | | | | | | |
| | AAV- | | | | | | | | | |
| | Cre, | | | | | | | | | |
| | WD | | | | | | | | | |
| 4769 | Gpam7f/f- | 37.54 | 1.94 | 5.17 | 110 | 68 | 226 | 117 | 6.90 | 223.68 |
| | satuated | | | | | | | | | |
| | AAV- | | | | | | | | | |
| | Cre, | | | | | | | | | |
| | WD | | | | | | | | | |

A NAFLD activity scoring system tailored to rodent histology was used to quantify steatosis and liver injury. This showed that KO livers had reduced macrovesicular steatosis, microvesicular steatosis, and hepatocyte hypertrophy (FIG. 4K). Liver triglyceride but not cholesterol levels were decreased in the WD fed KO group (FIGS. 4L and 4M), consistent with GPAM's role as a rate limiting enzyme in triglyceride synthesis. Interestingly, plasma cholesterol but not triglyceride was decreased in the KO group (FIG. 4N and 4O. Altogether, the data show that GPAM loss of function mutations lead to clonal expansions in NASH, and that GPAM is a promising therapeutic target in NASH.

### Experimental Methods and Subjects Used in the Examples

### Mouse Strains and Breeding

All mice were handled in accordance with the guidelines of the Institutional Animal Care and Use Committee at UT Southwestern. All experiments were done in an age and sex controlled fashion unless otherwise noted. All mice used in this study were male except for those in Figure S8F-J. C57BL/6 strain background mice were used for all experiments. Mboat7^{tm1a(KoMP)Wtsi}/H mice, obtained from KOMP, were originally generated by the Wellcome Trust Sanger Institute. Gpam^{f/f} mice were generated in the CRI Mouse Genome Engineering Core at UT Southwestern. In brief, CAS9 protein, synthetic sgRNA, and single-stranded DNA containing Gpam exon3 flanked by LoxP sites and homology arms, were co-injected into C57BL/6 mouse zygotes, which were then implanted into CD-1 mice. Genotyping and Sanger sequencing was used to confirm homologous recombination in the genome-edited pups. LSL-tdTomato (strain #007914) and Rosa-rtTA; TetO-Cas9 mice (#029415) were obtained from The Jackson Laboratory. Mice homozygous for both Rosa-rtTA and TetO-Cas9 were used to ensure a high Cas9 expression level in the liver. Western Diet (WD) used for NAFLD/NASH modeling is described in 45. It is composed of high fat solid food (ENVIGO #TD.120528) and high sugar water containing 23.1 g/L d-fructose (Sigma-Aldrich #F0127) and 18.9 g/L d-glucose (Sigma-Aldrich #G8270).

### Lineage Tracing in Floxed Mice

For whole-liver deletion of Mboat7 or Gpam, 5*10¹⁰ genomic copies of commercially produced AAV8-TBG-Cre (Addgene #107787) or control AAV8-TBG-GFP (Addgene #105535) in 100 μl saline was injected retro-orbitally into Mboat7^{f/f} or Gpam^{f/f} mice at 8 weeks of age. For mosaic deletion of Mboat7 or Gpam and Tomato labeling of hepatocytes, 0.125*10¹⁰ genomic copies of AAV8-TBG-Cre in 100 μl saline was injected retro-orbitally into Mboat7^{f/f}; LSL-tdTomato het, Gpam^{f/f}; LSL-tdTomato het, or control LSL-tdTomato het mice at 8 weeks of age. One week after injection of low dose AAV8-TBG-Cre, 7 mice from each group were collected to determine the initial Tomato labeling percentage in the liver. The remaining mice were divided into chow or WD groups and traced for another 4 or 6 months.

### Fluorescent Imaging and Image Processing

For fluorescent imaging, liver pieces were fixed in buffered formalin (Fisherbrand #245-685) for 24 h with gentle shaking at 4° C. and then transferred into 30% sucrose (w/v) solution for another 24 h with shaking at 4° C. The livers were then embedded and frozen in Cryo-Gel (Leica #39475237), and sectioned at a thickness of 16 μm. Images were taken using a Zeiss Axionscan Z1 system in the UTSW Whole Brain Microscopy Facility to visualize Tomato clones. To statistically analyze the percentage of Tomato+ cells, black and white fluorescent images were taken from the same slide using an Olympus IX83 microscope at 4× magnification. Two different fields were taken for each liver. The percentage of Tomato+ cells (bright areas) was analyzed using ImageJ.

### H&E, Immunohistochemistry (IHC), Immunofluorescence (IF), TUNEL, and Sirius Red Staining

Liver pieces were fixed in buffered formalin (Fisherbrand #245-685) for 24 h with gentle shaking at 4° C. and then transferred to 70% EtOH for another 24 h with shaking at 4° C. Paraffin embedding, liver sectioning (4 μm thickness), and H&E staining were performed at the UT Southwestern Tissue Management Shared Resource Core. IHC was performed as previously described. Briefly, paraffin-embedded sections were dewaxed in xylene and hydrated using ethanol gradients. The slides were then boiled in antigen retrieval buffer (10 mM sodium citrate, 0.05% Tween 20, pH 6.0) for 20 min and soaked in 3% hydrogen peroxide (in methanol) for 10 min. After blocking with 5% goat serum for 1 h at room temperature, the slides were incubated with primary antibody overnight at 4° C. After washing, the slides were incubated with secondary antibody at room temperature for 0.5 h. The secondary antibody was coupled with HRP using ABC-HRP Kit (Vector laboratories, #PK-6101). The slides were developed using the DAB Kit (Vector laboratories, #SK-4100). For IF staining, the following primary antibodies were used: RFP (Rockland #600-401-379, IF 1:500); Ki67 (Invitrogen #14-5698-82, IF 1:500); HNF4a (Abcam #ab41898, IF 1:500), and the following secondary antibodies were used: Goat anti-rat IgG (H&L) Alexa Fluor Plus 488 (Invitrogen #A-48262, IF 1:500); Donkey anti-rabbit IgG (H&L) Alexa Fluor 594 (Invitrogen #A-21207, IF 1:500); Goat anti-mouse IgG2a Alexa Fluor 647 (Invitrogen #A-21241, IF 1:500). IF was performed on paraffin embedded mouse liver sections using the same protocol as IHC except that secondary antibodies were substituted by Alexa Fluor conjugated antibodies. TUNEL staining was performed on paraffin embedded liver sections using In Situ Cell Death Detection Kit, Fluorescein (Roche #C755B40) according to the manufacturer's protocol. Sirius Red staining was performed on paraffin embedded liver sections using the Picro Sirius Red Staining Kit (Abcam #ab150681) according to the manufacturer's protocol. QuPath software was used to quantify TUNEL staining and IHC staining of Ki67. ImageJ was used to quantify Sirius Red staining.

### Plasma and Liver Metabolic Assays

Blood was taken using heparinized tubes from the inferior vena cava immediately after sacrificing the mouse, and then transferred into 1.5 ml tubes and centrifuged at 2000 g for 15 min at 4° C. The supernatant after centrifugation (plasma) was analyzed for AST, ALT, cholesterol, and triglyceride (Manufacturer's Reference Numbers 8433815, 1655281, 1669829, and 1336544, respectively) using a fully automated OCD Vitros 350 dry chemistry analyzer following the protocols provided by the reagent kit manufacturer (Ortho Clinical Diagnostics, Raritan, NJ) at the UT Southwestern Metabolic Phenotyping Core. 100-150 mg of liver per mouse was weighed and used for lipid extraction and quantification at the UT Southwestern Metabolic Phenotyping Core. Briefly, flash frozen tissue samples were homogenized with 2:1 chloroform:methanol mixture (v/v) using a multiplexed automatic tissue disruptor (TissueLYser II, Qiagen, Germantown, MD). The organic extract was transferred to a 5 ml graduated flask and the total volume was brought up to 5 ml. Total cholesterol and triacylglycerol analyses were performed in triplicate using 100 μl and 25 μl of lipid extracts, respectively. Total cholesterol and triacylglycerol concentrations were determined by commercial enzymatic colorimetric assays following the protocols described by the manufacturer (INFINITY™ Cholesterol Liquid Stable Reagent #TR13421; INFINITY™ Triglycerides Liquid Stable Reagent #TR22421; Matrix plus™ Chemistry Reference Kit #NC9592194).

### MOSAICS Reagent Construction

The MOSAICS plasmid uses the pX602 plasmid as a backbone. The sequence between the two AAV ITRs were removed using the Nsil and Notl restriction enzymes. The following fragments were cloned between the two AAV ITRs: the first SB100 binding IR, a U6 driven sgRNA scaffold, a CAG promoter driven SB100-P2A-Cre fusion cDNA with a beta-globin poly(A) signal, and the second SB100 binding IR. For library construction, mouse candidate genes for all of the in vivo screens were generated by using the mouse homologs of the human genes. A few genes were not included in the mouse gene lists due to the lack of a homolog or because they were known tumor suppressor genes. The individual sgRNA sequences corresponding to mouse candidate genes were extracted from the Brie library or obtained from the GUIDES server, and synthesized by CustomArray. Most genes had 5 distinct sgRNAs, 4 from Brie and 1 from GUIDES. A few genes had 4 targeting sgRNAs due to the overlap of sgRNA sequences from Brie and GUIDES. See Table 5 for the sgRNA sequences for GPAM. The library construction protocol was as well established in the field. Briefly, synthesized oligonucleotide libraries were amplified by PCR, purified using a PCR Purification Kit (Qiagen, #28104), and assembled in Bsal digested MOSAICS vector using DNA Assembly Kit (NEB, #E5520A). 1 μl of the assembled vector was then electroporated into 25 μl competent cells (Lucigen, #60052-2). After recovery in SOC medium for 1 h, bacteria were spread on a 245*245 mm LB agar plate and incubated at 37° C. overnight. The bacteria were then harvested for plasmid preparation using the HiSpeed Plasmid Maxi Kit (Qiagen, #12663). Each sgRNA maintained a >1000-fold representation during construction. For individual sgRNA cloning, forward and reverse primers were annealed and fused to Bsal digested MOSAICS plasmid using T4 ligase. See Table 5 for the primers associated with the sgRNAs.

TABLE 4

| | |
|---|---|
| Sequence | SEQ ID NO |

sgRNA target sequence

| | | |
|---|---|---|
| GPAM1 | AGTTTCTGCCGGTTGCACTG | 166 |
| GPAM2 | GGGTCAACTCGAGATGGTCA | 167 |
| GPAM3 | CAAGAGCGAGACGTCCATAA | 168 |
| GPAM4 | TCGTATGATCGCATAATCGA | 169 |
| GPAM5 | AAACTACGGCTACGTCCGAG | 170 |

Primers

| | | |
|---|---|---|
| GPAM-F | CCGCGAGTAACTGTGGTATTTA | 171 |
| GPAM-R | GGTTGAGAACCACTGAGTTAAGA | 172 |
| GPAM_qPCR_F1 | ATCCCATCTCTGGGTTTGCG | 173 |
| GPAM_qPCR_R1 | CCCTTATGGACGTCTCGCTC | 174 |
| GPAM_qPCR_F2 | GAGGACTGGGTTGACTGTGG | 175 |
| GPAM_qPCR_R2 | TTCCGCAAACCCAGAGATGG | 176 |

### AAV Production and Purification

AAV8 was produced using AAV-Pro 293T cells (Takara #632273) cultured in one or more 15 cm dishes. Cells were plated one day before transfection at 50% confluence, which would allow the cells to reach 80-90% confluence the next day. For transfection of one 15 cm dish, 10 μg MOSAICS vector, 10 μg pAAV2/8 (Addgene #112864) and 20 μg pAdDeltaF6 (Addgene #112867) plasmids were mixed with 1 ml Opti-MEM medium in one tube. In another tube, 160 μl PEI solution (1 mg/ml in water, pH7.0, powder from ChemCruz #sc-360988) was mixed with 1 ml Opti-MEM medium. The solutions from both tubes were then mixed and incubated for 10 min before adding to cell culture. 48 h after transfection, the cells were scraped off the dish and collected by centrifugation at 500 g for 10 min. The supernatant was disinfected and discarded, and the cell pellets were lysed in 1.5 ml/15 cm dish lysis buffer (PBS supplemented with NaCl powder to final concentration of 200 mM, and with CHAPS powder to final concentration of 0.5% (w/v)). The cell suspension was put on ice for 10 min with intermittent vortexing, and then centrifuged at 20,000 g for 10 min at 4° C. The supernatant containing the AAV was collected. To set up the gravity column for AAV purification, 0.5 ml of AAV8-binding slurry beads (ThermoFisher #A30789), enough to purify AAV from three 15 cm dishes, was loaded into an empty column (Bio-Rad #731-1550). After the beads were tightly packed at the bottom, they were washed with 5 ml of wash buffer (PBS supplemented with NaCl powder to a final concentration of 500 mM). The supernatant containing AAV was then loaded onto the column. After all of the supernatant flowed through, the beads were washed with 10 ml wash buffer twice. The AAV was then eluted with 3 ml elution buffer (100 mM glycine, 500 mM NaCl in water, pH 2.5) and the eluate was immediately neutralized with 0.12 ml 1M Tris-HCl (pH 7.5-8.0). The AAV was concentrated by centrifugation at 2000 g for 3-5 min at 4° C. using an 100 k Amicon Ultra Centrifugal Filter Unit (Millipore #UFC810024). After centrifugation, the volume of AAV should be equal to or less than 0.5 ml. The concentrated AAV was diluted with 4-5 ml AAV dialysis buffer (PBS supplemented with powders to final concentrations of 212 mM NaCl and 5% sorbitol (w/v)) and centrifuged at 2000 g for 3-5 min at 4° C. The dilution and centrifugation processes were repeated 3 times. The final concentrated AAV was transferred into a 1.5 ml tube and centrifuged at 20,000 g for 5 min to remove debris. The supernatant was aliquoted, flash frozen using liquid nitrogen, and stored at −80° C.

Genomic DNA Extraction, sgRNA Amplification, and Amplicon Library Construction

To extract genomic DNA containing the integrated sgRNA, the entire liver (except a small piece used for sectioning and H&E staining) was minced into about 1 mm³ pieces using a blade and weighed. Small nodules observed in some epigenetic factor screening livers given WD (<=3 nodules per liver in 5 out of 8 livers) were excluded from samples being processed for genomic DNA extraction. Minced liver in two volumes (w/v) of homogenizing buffer (100 mM NaCl, 25 mM EDTA, 0.5% SDS, 10 mM Tris-HCl, pH 8) was transferred into a glass Wheaton Dounce Tissue Grinder and stroked 50 times or until no bulk tissues were seen. After homogenizing, 200 μl chow fed liver lysate or 300 μl WD fed liver lysate was transferred to a 15 ml tube for genomic DNA extraction using the Blood & Cell Culture DNA Midi Kit (Qiagen #13343) according to the manufacturer's protocol. The remaining lysates were frozen in −80° C. as backup samples. Briefly, 10 ml Buffer G2 from the kit, 100 μl Proteinase K (Roche #03115828001, or Proteinase K from the Qiagen kit) and 100 μl RNase A (Invitrogen #12091-021) were added to the 15 ml tube containing the lysate and digested in a 50° C. water bath overnight. The next day, the tubes were centrifuged at 4000 g for 10 min and the lipid layer on the top was discarded. The remaining supernatant was loaded on the column, washed, and genomic DNA elution/precipitation were performed according to the manufacturer's protocol. The precipitated DNA was resuspended in 100 μl 10 mM Tris (pH 8.0) and shaken on a 55° C. shaker for 2 h to help it dissolve. For amplicon library preparation, 5 μg genomic DNA, 5 μl general forward primer mix (5 μM), 5 pl barcode specific reverse primer (5 μM), 1 μl Q5 DNA polymerase, 10 μl Q5 buffer, 10 μl HighGC buffer, 1 μl dNTP, and water was mixed for a 50 μl PCR reaction, and two reactions were made for each genomic DNA sample. The PCR cycle was 95° C. 3 min-(95° C. 30 s−60° C. 30 s−72° C. 20 s)*n−72° C. 2 min. The PCR cycle number was pre-optimized using the same PCR reactions with a smaller volume. The cycle numbers that gave a weak but sharp band on the DNA gel were used. In the final PCR reaction, 23 cycles were used for preparing the NASH gene, transcription factor, and epigenetic factor screens, and 30 cycles were used for preparing the guide mini-pool validation screen. After PCR, the two tubes of reactions with the same genomic DNA template were combined (total 100 μl) and 70 μl was resolved on a DNA gel. The 250 bp band corresponding to the amplicon was cut and purified using the QIAquick Gel Extraction Kit (Qiagen #28704). The DNA concentration was determined using Qubit kit (Invitrogen #Q32853) and high-throughput sequencing was performed using an Illumina NextSeq500 system at the CRI at UT Southwestern Sequencing Facility.

### Bioinformatic Analysis of MOSAICS Screening Results

The reads from the sequencing of amplicon libraries described above were trimmed with cutadapt (version 1.9.1) to remove the excessive adaptor sequences so that only the sgRNA sequences were retained. The 5′ sequences were trimmed with the options -O 32 —discard-untrimmed -g CTTTATATATCTTGTGGAAAGGACGAAACACCG. The 3′ sequences were trimmed with the options -O 12 -a GTTTTAGAGCTAGAAATAGCA. The abundance of each sgRNA was calculated with the count function in MAGeCK (version 0.5.6) with the default option. The trimmed fastq files were assigned to chow-fed and WD-fed groups and uploaded together with library files containing sgRNA sequences and targeted gene names to a server preloaded with MAGeCK. The enrichment of each sgRNA was calculated with the test function in MAGeCK.

### Transcriptomic Analysis of Human NASH Livers

Dataset 1 (NAFLD/NASH cohort): The RNA-Seq transcriptome profiles of biopsied liver tissues (GEO: GSE1309701 were downloaded and analyzed 72 NAFLD/NASH patients with a range of disease severities (NASH activity scores of 1 to 6) and relevant histological features, i.e., steatosis, inflammation, fibrosis, and hepatocyte ballooning. The raw sequence reads were aligned to the GENCODE human reference genome (GRCh37, p13) using the STAR aligner (ver 2.6.1b), and gene-level count data were generated by the feature Counts function in the Subread package (ver 1.6.1), and the GENCODE genome annotation (GRCh37, v19). The count data were normalized using “Relative Log Expression” normalization (RLE) implemented in the DESeq2 package Dataset 2 (HCV cirrhosis cohort): The microarray gene expression profile of formalin-fixed needle biopsy specimens from the livers of 216 patients with hepatitis C-related early-stage (Child-Pugh class A) cirrhosis (GEO: GSE156540) was analyzed. This cohort was prospectively followed for a median of 10 years at an Italian center with relevant time event outcomes collected, including child, death, HCC and decomposition. Male and female patients were included in these studies.

### Quantification and Statistical Analysis

The data in most panels reflect multiple experiments performed on different days using mice derived from different litters. Variation in all panels is indicated using standard deviation presented as mean±SD. Two-tailed unpaired Student's t-tests were used to test the significance of differences between two groups. Statistical significance is displayed as ns (not significant, or p>=0.05), * (p<0.05), ** (p<0.01), *** (p<0.001), **** (p<0.0001) unless specified otherwise. Image analysis for quantification was blinded.

What is claimed is:

1. A composition comprising a nucleic acid molecule that downregulates expression of GPAM or a variant thereof.

2. The composition of claim 1, wherein the nucleic acid is a siRNA, a cluster regularly interspaced short palindromic repeats (CRISPR) related nucleic acid, a single guide RNA (sgRNA), a CRISPR-RNA (crRNA), a trans-activating crRNA (tracrRNA), a plasmid DNA (pDNA), or viral vector.

3. The composition of claim 2, wherein the nucleic acid is a small interfering RNA (siRNA) molecule or encodes a siRNA molecule.

4. A composition comprising a plasmid or a viral vector, wherein the plasmid or the viral vector comprises a nucleic acid encoding the siRNA molecule of claim 3.

5. The composition of any one of claim 3 or 4, wherein the siRNA molecule comprises a nucleotide sequence that is 2 to 30 nucleotides in length and is at least 80% homologous to at least 2 to 30 contiguous nucleotides of a human GPAM cDNA sequence.

6. The composition of claim 5, wherein the human GPAM cDNA sequence is SEQ ID NO: 1.

7. The composition of any one of claim 3 or 4, wherein the siRNA molecule targets the open reading frame or the 5′ or 3′ UTRs of the GPAM gene.

8. The composition of any one of claims 3-7, wherein the siRNA molecule comprises at least one sense sequence, at least one antisense sequence, or at least one sense sequence and at least one antisense sequence.

9. The composition of any one of claims 3-8, wherein the siRNA molecule comprises a nucleotide sequence of SEQ ID NOs: 2-165, or any combination thereof.

10. The composition of claim 9, wherein the at least one sense sequence comprises SEQ ID NOs: 2-83.

11. The composition of claim 9, wherein the at least one antisense sequence comprises SEQ ID NOs: 84-165.

12. The composition of claim 2, wherein the nucleic acid is a sgRNA or encodes an sgRNA.

13. A composition comprising a plasmid or a viral vector, wherein the plasmid or the viral vector comprises a first nucleic acid encoding the sgRNA molecule of claim 12 and optionally a second nucleic acid encoding an RNA guided nuclease.

14. The composition of any one of claim 12 or 13, wherein the sgRNA target sequence is at least about 80% identical to any one of SEQ ID NO: 177-276.

15. The composition of claim 13, wherein the RNA guided nuclease is a Cas endonuclease.

16. The composition of any one of claims 3-11, wherein the siRNA molecule specifically downregulates gene expression of at least one variant of GPAM.

17. The composition of any one of claims 12-14, wherein the sgRNA molecule specifically downregulates gene expression of at least one variant of GPAM.

18. The composition of any one of claim 16 or 17, wherein the at least one variant of GPAM is associated with a liver disease.

19. The composition of claim 18, wherein the liver disease comprises fatty liver disease (FLD), alcohol-related liver disease (ARLD), non-alcoholic fatty liver disease (NAFLD), non-alcoholic steatohepatitis (NASH), end stage liver disease (cirrhosis) from any etiology, liver cancer, or any combination thereof.

20. The composition of any one of claims 16-19, wherein the at least one variant of GPAM comprises rs10787429 C>T, rs7096937 T>C, rs11446981 T>TA, rs7898213 T>C, rs2792759 C>T, rs2297991 T>C, rs1129555 A>G, rs2254537 T>A, or any combination thereof.

21. The composition of any one of claims 1-3, wherein the nucleic acid molecule is conjugated to least one targeting ligand.

22. The composition of claim 21, wherein the at least one targeting ligand comprises a liver targeting ligand.

23. The composition of claim 22, wherein the liver targeting ligand comprises at least one N-acetylgalactosamine (GalNAc) conjugate.

24. The composition of claim 23, wherein the nucleic acid molecule is conjugated to about one to about three GalNAc conjugates.

25. The composition of any one of claim 1-3 or 12, wherein the nucleic acid molecule comprises at least one chemical modification.

26. The composition of any one of claim 25, wherein the nucleic acid molecule comprises a modification at least one ribosugar moiety of its nucleotide sequence.

27. The composition of claim 26, wherein at least one ribosugar moiety is modified with 2 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, 2′-C-allyl, or any combination thereof.

28. The composition of either claim 26 or claim 27, wherein less than about 10% to about 70% of ribosugar moieties of the total nucleotide sequence is modified.

29. A pharmaceutical composition comprising any one of the compositions of claims 1-28 and at least one pharmaceutically acceptable carrier.

30. The pharmaceutical composition of claim 29, further comprising a nanoparticle.

31. The pharmaceutical composition of either claim 29 or claim 30, further comprising a lipid.

32. A method of for treating a subject in need thereof, the method comprising administrating a therapeutically effective amount of the composition of any one of claims 1-28 or the pharmaceutical composition of any one of claims 29-31.

33. The method of claim 32, wherein the subject in need thereof, is a human subject having or suspected of having a liver disease.

34. The method of claim 33, wherein the liver disease comprises fatty liver disease (FLD), alcohol-related liver disease (ARLD), non-alcoholic fatty liver disease (NAFLD), non-alcoholic steatohepatitis (NASH), end stage liver disease (cirrhosis) from any etiology, liver cancer, or any combination thereof.

35. The method of any one of claims 32-34, wherein the method of administering comprises parenteral administration.

36. The method of any one of claims 31-34, wherein administration of a therapeutically effective amount of the composition of any one of claims 1-27 or the pharmaceutical composition of any one of claims 28-30 increases life expectancy of the subject compared to an untreated subject with identical disease condition and predicted outcome.

37. The method of any one of claims 32-36, wherein administration of a therapeutically effective amount of the composition of any one of claims 1-28 or the pharmaceutical composition of any one of claims 29-31 increases liver function of the subject compared to an untreated subject with identical disease condition and predicted outcome.

38. The method of any one of claims 32-37, wherein administration of a therapeutically effective amount of the composition of any one of claims 1-28 or the pharmaceutical composition of any one of claims 29-31 attenuates liver fibrosis in the subject compared to an untreated subject with identical disease condition and predicted outcome.

39. The method of any one of claims 32-38, wherein administration of a therapeutically effective amount of the composition of any one of claims 1-28 or the pharmaceutical composition of any one of claims 29-31 prevents additional liver fibrosis in the subject compared to an untreated subject with identical disease condition and predicted outcome.

40. A kit comprising
(i) a container holding the composition of any one of claims 1-28 or the pharmaceutical composition of any one of claims 29-31;
(ii) a pharmaceutical administrative means; and
(iii) an instruction.
